SEQUENCE LISTING (25 sequences)

SEQ ID NO: 1 - protein, 24 aa, Artificial Sequence
  Description of Artificial Sequence Synthetic peptide
RRWVRRVRRVWRRVVRVVRRWVRR

SEQ ID NO: 2 - protein, 14 aa, Artificial Sequence
  Description of Artificial Sequence Synthetic peptide
IRRRRRRIRRRRRR

SEQ ID NO: 3 - protein, 21 aa, Artificial Sequence
  Description of Artificial Sequence Synthetic peptide
IRRRIRRIRRRIRRIRRRIRR

SEQ ID NO: 4 - protein, 21 aa, Artificial Sequence
  Description of Artificial Sequence Synthetic peptide
IRRIIRRIRRIIRRIRRIIRR

SEQ ID NO: 5 - protein, 21 aa, Artificial Sequence
  Description of Artificial Sequence Synthetic peptide
VWRWVRRVWRWVRRVWRWVRR

SEQ ID NO: 6 - protein, 14 aa, Artificial Sequence
  Description of Artificial Sequence Synthetic peptide
VWRWVRRVWRWVRR

SEQ ID NO: 7 - protein, 14 aa, Artificial Sequence
  Description of Artificial Sequence Synthetic peptide
VVRVVRRVVRVVRR

SEQ ID NO: 8 - protein, 21 aa, Artificial Sequence
  Description of Artificial Sequence Synthetic peptide
VVRVVRVVVRVVRVVVRVVRV

SEQ ID NO: 9 - protein, 11 aa, Artificial Sequence
  Description of Artificial Sequence Synthetic peptide
RSRVVRSWSRV

SEQ ID NO: 10 - protein, 84 aa, Artificial Sequence
  Description of Artificial Sequence Synthetic polypeptide
RFVRRVRRFVRRVRRFVRRVRRFVRRVRRFVRRVRRFVRRVRRFVRRVRRFVRRVRRFVRRVRRFVRRVRRFVRRVRRFVRRVR

SEQ ID NO: 11 - protein, 20 aa, Artificial Sequence
  Description of Artificial Sequence Synthetic peptide
RRTYSRSRRTYSRSRRTYSR

SEQ ID NO: 12 - protein, 28 aa, Artificial Sequence
  Description of Artificial Sequence Synthetic peptide
KVVSSIIEIISSVVKVVSSIIEIISSVV

SEQ ID NO: 13 - protein, 20 aa, Artificial Sequence
  Description of Artificial Sequence Synthetic peptide
KKTHTKTKKTHTKTKKTHTK

SEQ ID NO: 14 - protein, 21 aa, Artificial Sequence
  Description of Artificial Sequence Synthetic peptide
VVRVVRRVVRVVRRVVRVVRR

SEQ ID NO: 15 - protein, 12 aa, Artificial Sequence
  Description of Artificial Sequence Synthetic peptide
RVVRVVRRVVRR

SEQ ID NO: 16 - protein, 12 aa, Artificial Sequence
  Description of Artificial Sequence Synthetic peptide
RVVRVVRRWVRR

SEQ ID NO: 17 - protein, 12 aa, Artificial Sequence
  Description of Artificial Sequence Synthetic peptide
RWWRWWRRWWRR

SEQ ID NO: 18 - protein, 18 aa, Artificial Sequence
  Description of Artificial Sequence Synthetic peptide
WRRWWRRWWRWWRRWWRR

SEQ ID NO: 19 - protein, 24 aa, Artificial Sequence
  Description of Artificial Sequence Synthetic peptide
RRVVRRVRRVVRRVVRVVRRVVRR

SEQ ID NO: 20 - protein, 24 aa, Artificial Sequence
  Description of Artificial Sequence Synthetic peptide
RRWWRRWRRWWRRWWRWWRRWWRR

SEQ ID NO: 21 - protein, 36 aa, Artificial Sequence
  Description of Artificial Sequence Synthetic polypeptide
VRRVVRRVVRVVRRVVRRVRRVVRRVVRVVRRVVRR

SEQ ID NO: 22 - protein, 36 aa, Artificial Sequence
  Description of Artificial Sequence Synthetic polypeptide
VRRVWRRVVRVVRRWVRRVRRVWRRVVRVVRRWVRR

SEQ ID NO: 23 - protein, 42 aa, Artificial Sequence
  Description of Artificial Sequence Synthetic polypeptide
RRVVRRVRRVVRRVVRVVRRVVRRVRRVVRRVVRVVRRVVRR

SEQ ID NO: 24 - protein, 48 aa, Artificial Sequence
  Description of Artificial Sequence Synthetic polypeptide
RVVRVVRRVVRRVRRVVRRVVRVVRRVVRRVRRVVRRVVRVVRRVVRR

SEQ ID NO: 25 - protein, 48 aa, Artificial Sequence
  Description of Artificial Sequence Synthetic polypeptide
RVVRVVRRWVRRVRRVWRRVVRVVRRWVRRVRRVWRRVVRVVRRWRVV

(19) United States
(12) Patent Application Publication
(10) Pub. No.: US 2023/0106943 A1
(43) Pub. Date: Apr. 6, 2023

(54) ENGINEERED ANTIMICROBIAL PEPTIDES AND USAGE THEREOF

(71) Applicant: Peptilogics, Inc., Pittsburgh, PA (US)

(72) Inventors: Jonathan D. STECKBECK, Cranberry Township, PA (US); Bradd N. PICONE, Jefferson Hills, PA (US); Despina X. DOBBINS, Clearwater Beach, FL (US)

(21) Appl. No.: 17/730,042

(22) Filed: Apr. 26, 2022

Related U.S. Application Data
(60) Provisional application No. 63/245,770, filed on Sep. 17, 2021.

(51) Int. Cl. A61K 38/17; A61K 38/16; A61K 38/10

(52) U.S. Cl. CPC A61K 38/1729; A61K 38/16; A61K 38/10

(57) ABSTRACT

Provided in this disclosure are pharmaceutical formulations comprising antimicrobial peptides with enhanced stability. Further provided herein are methods of treating or preventing an infection comprising administering pharmaceutical formulations comprising antimicrobial peptides when administered to a subject.

### CROSS-REFERENCE

This application claims the benefit of U.S. Provisional Application No. 63/245,770, filed on Sep. 17, 2021, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Jun. 8, 2022, is named 48615-715 201 SL.txt and is 9,550 bytes in size.

### SUMMARY

Disclosed herein are pharmaceutical formulations comprising: a peptide or pharmaceutically acceptable salt thereof comprising at least about 70% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg- Arg-Val-Val-Arg-Arg (SEQ ID NO: 24); or Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val- Arg-Arg-Trp-Arg-Val-Val (SEQ ID NO: 25); and at least one pharmaceutically acceptable excipient; wherein the pharmaceutical formulation has a pH of about 3.5 to about 5.5; and wherein the pharmaceutical formulation comprises at most 5% by weight of at least one impurity as measured by high-performance liquid chromatography (HPLC) when stored for at least 50 days at 40° C.

In some embodiments, pharmaceutical formulation can be stable for at least about 3 months at a temperature of about 40° C. In some embodiments, pharmaceutical formulation can be stable for at least about 6 months at a temperature of about 40° C. In some embodiments, pharmaceutical formulation can be stable for at least about 9 months at a temperature of about 40° C. In some embodiments, pharmaceutical formulation can comprise a pH of about 5.0. In some embodiments, pharmaceutical formulation can comprise a pH of about 4.5. In some embodiments, pharmaceutical formulation can comprise a pH of about 4.9. In some embodiments, pharmaceutical formulation can comprise at most 3.5% by weight of said at least one impurity as measured by HPLC. In some embodiments, pharmaceutical formulation can comprise at most 2% by weight of said at least one impurity as measured by HPLC. In some embodiments, pharmaceutical formulation can comprise at most 1.5% by weight of said at least one impurity measured by HPLC. In some embodiments, pharmaceutical formulation can comprise at most 3% by weight of said at least one impurity as measured by UV-Vis spectroscopy. In some embodiments, the peptide is present at a concentration of about 10 mg/mL to about 100 mg/mL. In some embodiments, the peptide is present at a concentration at about 15 mg/mL, about 30 mg/mL, about 40 mg/mL, about 70 mg/mL, or about 80 mg/mL. In some embodiments, the peptide comprises at least about 90% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1). In some embodiments, wherein the peptide comprises Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1). In some embodiments, the excipient comprises an isotonicity agent. In some embodiments, the isotonicity agent is sodium chloride. In some embodiments, pharmaceutical formulation can further comprise a pH adjustment agent. In some embodiments, the pH adjustment agent comprises hydrochloric acid, sodium hydroxide, or any combination thereof. In some embodiments, pharmaceutical formulation can comprise further comprising a pH buffering agent. In some embodiments, the pH buffering agent is selected from the group consisting of sodium hydrogen phosphate, sodium dihydrogen phosphate, potassium dihydrogen phosphate, potassium hydrogen phosphate, glycine, tris(hydroxymethyl)aminomethane, and any combination thereof. In some embodiments, the formulation is free of a buffering agent. In some embodiments, pharmaceutical formulation can comprise an osmolality of at least about 30 milliosmoles per kilogram (mOsm/kg) to at least about 800 mOsm/kg. In some embodiments, pharmaceutical formulation can comprise an osmolality of about 100 mOsm/kg to about 500 mOsm/kg. In some embodiments, pharmaceutical formulation can have an increase in solubility. In some embodiments, the increase in solubility is a function of the pH. In some embodiments, pharmaceutical formulation can be in the form of a tablet, a liquid, a syrup, an oral formulation, an intravenous formulation, an intranasal formulation, an ocular formulation, an otic formulation, a subcutaneous formulation, a suppository, and any combination thereof.

Disclosed herein are kits comprising (i) the pharmaceutical formulation disclosed herein; (ii) an aqueous carrier, wherein the aqueous carrier is sterile water for injection; (iii) a mixing container; and (iv) instructions for use. In some embodiments, the kit further can comprise (v) a second aqueous carrier, wherein the second aqueous carrier is aqueous sodium bicarbonate.

Disclosed herein are methods of preventing or treating an infection in a subject in need thereof, wherein the method comprising locally administering of the pharmaceutical formulation described herein to a site of infection, wherein administration comprises washing, irrigating, debridement, or a combination thereof of the site of infection, thereby preventing or treating the infection. In some embodiments, the infection is periprosthetic joint infection. In some embodiments, the infection is a bacterial infection, wherein the bacterial species is selected from the group consisting of Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, and any combination thereof. Disclosed herein are methods of preventing or treating an infection in a subject in need thereof, wherein the method comprising locally administering the kit described herein to a site of infection, wherein administration comprises washing, irrigating, debridement, or a combination thereof of the site of infection, thereby preventing or treating the infection.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates an exemplary HPLC chromatograph of SEQ ID NO: 1 prior to storage in solid form.

FIG. 2 illustrates an exemplary HPLC chromatograph of SEQ ID NO: 1 after 3 years of storage at −20° C.

FIG. 3 illustrates an exemplary HPLC chromatograph of solid SEQ ID NO: 1.

FIG. 4 illustrates an exemplary HPLC chromatograph of SEQ ID NO: 1 after 7 days at 40° C. and after 54 days at 40° C.

FIG. 5 illustrates an exemplary overlaid HPLC chromatograph of SEQ ID NO: 1 and impurities at 0 day (bottom) and after 3 months (top) stored in PBS buffer at pH of 7.4. Impurity peaks are circled on the 3 months graph.

FIG. 6 illustrates an exemplary overlaid HPLC chromatograph of SEQ ID NO: 1 and impurities at 0 day (bottom) and after 3 months (top) stored in saline at pH of 5.0. Impurity peaks are circled on the 3 months graph.

FIG. 7 illustrates an exemplary HPLC chromatograph of 1.0 mg/mL SEQ ID NO: 1 at after 3 months stored at 5° C. in saline at pH of 5.0.

FIG. 8 illustrates an exemplary HPLC chromatograph of 1.0 mg/mL SEQ ID NO: 1 at after 3 months stored at room temperature in saline at pH of 5.0.

FIG. 9 illustrates an exemplary HPLC chromatograph of 1.0 mg/mL SEQ ID NO: 1 at after 3 months stored at −20° C. in saline at pH of 5.0.

FIG. 10 illustrates an exemplary HPLC chromatograph of 1.0 mg/mL SEQ ID NO: 1 at after 3 months stored at 5° C. in PBS solution at pH of 7.4.

FIG. 11 illustrates an exemplary HPLC chromatograph of 1.0 mg/mL SEQ ID NO: 1 at after 3 months stored at room temperature in PBS solution at pH of 7.4.

FIG. 12 illustrates an exemplary HPLC chromatograph of 1.0 mg/mL SEQ ID NO: 1 at after 3 months stored at −20° C. in PBS solution at pH of 7.4.

FIG. 13 illustrates an exemplary HPLC chromatograph of 1.0 mg/mL SEQ ID NO: 1 at after 3 months stored at 5° C. in tromethamine buffer at pH of 7.4.

FIG. 14 illustrates an exemplary HPLC chromatograph of 1.0 mg/mL SEQ ID NO: 1 at after 3 months stored at room temperature in tromethamine buffer at pH of 7.4.

FIG. 15 illustrates an exemplary HPLC chromatograph of 1.0 mg/mL SEQ ID NO: 1 at after 3 months stored at −20° C. in tromethamine buffer at pH of 7.4.

FIG. 16 illustrates an exemplary HPLC chromatograph of 1.0 mg/mL SEQ ID NO: 1 at after 3 months stored at 5° C. in glycine buffer at pH of 7.4.

FIG. 17 illustrates an exemplary HPLC chromatograph of 1.0 mg/mL SEQ ID NO: 1 at after 3 months stored at room temperature in glycine buffer at pH of 7.4.

FIG. 18 illustrates an exemplary HPLC chromatograph of 1.0 mg/mL SEQ ID NO: 1 at after 3 months stored at −20° C. in glycine buffer at pH of 7.4.

FIG. 19 plots % area of exemplary SEQ ID NO: 1 solutions at different pH and stored at 40° C. over period of time.

### DETAILED DESCRIPTION

### Pharmaceutical Formulation

### Peptides

The development of antimicrobial agents is paramount due to the emergence of pathogens resistant to traditional antimicrobial compounds. Disclosed herein are peptides that comprise antimicrobial, antiviral, antifungal or antitumor activity when administered to a subject. A peptide described herein can be used to disrupt the integrity of a membrane by (a) binding to a negatively charged surface on a membrane; and/or (b) integrating into a membrane. The ability of a peptide disclosed herein to bind to a negatively charged surface on a membrane and/or integrate into a membrane can allow a peptide to act as a toxic agent to cells with a negatively charged surface by disrupting membrane integrity. In other embodiments, a peptide disclosed herein can have anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, and/or protozoicidal properties.

The methods of treating a disease or condition described herein can be by administering to a subject a peptide or formulation containing a peptide as disclosed therein. For example, a peptide or formulation comprising a peptide described herein can be administered as an antimicrobial agent in order to at least partially inhibit the growth of a pathogen, such as bacteria, through disruption of the structural integrity of the bacterial cell membrane. A peptide described herein can be screened for broad spectrum activity against a variety of pathogens for broad utility when administered to a subject.

An antimicrobial peptide described herein can also be used as a means to produce an antimicrobial film for coating a device. In some embodiments, the peptides disclosed herein can be used to coat the interior and/or exterior of a medical device, for example, an implantable medical device. The coating of a device with a peptide disclosed herein can reduce the growth and proliferation of cells, bacteria, fungi or virus on a surface coated with a peptide. In some embodiments, coating an implantable medical device with a peptide disclosed herein can reduce the risk of an infection to a subject upon implanting the medical device in a subject.

It is further envisaged that a peptide described herein or formulation comprising a peptide described herein can be included in a kit. The kit can be utilized, for example, by a subject or healthcare professional to coat a device or to treat a condition or disease described herein.

The antimicrobial peptides may be derived from, and are analogs of, the LLP-1 peptide parent sequence corresponding to amino acids 828- 856 of the HIV-1 viral isolate HXB2R Env, (see Table 1 below). The antimicrobial activity of other LLP-1 peptide analogues has been previously described (see, Tencza et al., 1999, Journal of Antimicrobial Chemotherapy 44:33-41, U.S. Pat. No. 5,714,577 of Montelaro et al. and U.S. Pat. No. 5,945,507 of Montelaro et al., the disclosures of which are incorporated herein by reference). The antimicrobial peptides may be LLP-1 analogs having modifications based on the following principles: (i) optimizing amphipathicity, (ii) substituting arginine (Arg) on the charged face and/or valine (Val) or tryptophan (Trp) on the hydrophobic face with another amino acid, and (iii) increasing peptide length; see Table 1). Amino acid sequences are provided, left-to-right, from their N-terminus to their C-terminus in 1 letter designations and 3 letter designations.

TABLE 1
Antimicrobial Peptides

| SEQ ID | |
|---|---|
| NO: | Amino Acid Sequence |
| 1 | RRWVRRVRRVWRRVVRVVRRWVRR |
| | Arg-Arg-Trp-Val-Arg-Arg-Val-Arg-Arg-Val-Trp-Arg-Arg-Val-Val-Arg-Val- |
| | Val-Arg-Arg-Trp-Val-Arg-Arg |
| 2 | IRRRRRRIRRRRRR |
| | Ile-Arg-Arg-Arg-Arg-Arg-Arg-Ile-Arg-Arg-Arg-Arg-Arg-Arg |
| 3 | IRRRIRRIRRRIRRIRRRIRR |
| | Ile-Arg-Arg-Arg-Ile-Arg-Arg-Ile-Arg-Arg-Arg-Ile-Arg-Arg-Ile-Arg-Arg-Arg- |
| | Ile-Arg-Arg |
| 4 | IRRIIRRIRRIIRRIRRIIRR |
| | Ile-Arg-Arg-Ile-Ile-Arg-Arg-Ile-Arg-Arg-Ile-Ile-Arg-Arg-Ile-Arg-Arg-Ile-Ile- |
| | Arg-Arg |
| 5 | VWRWVRRVWRWVRRVWRWVRR |
| | Val-Trp-Arg-Trp-Val-Arg-Arg-Val-Trp-Arg-Trp-Val-Arg-Arg-Val-Trp-Arg-Trp- |
| | Val-Arg-Arg |
| 6 | VWRWVRRVWRWVRR |
| | Val-Trp-Arg-Trp-Val-Arg-Arg-Val-Trp-Arg-Trp-Val-Arg-Arg |
| 7 | VVRVVRRVVRVVRR |
| | Val-Val-Arg-Val-Val-Arg-Arg-Val-Val-Arg-Val-Val-Arg-Arg |
| 8 | VVRVVRVVVRVVRVVVRVVRV |
| | Val-Val-Arg-Val-Val-Arg-Val-Val-Val-Arg-Val-Val-Arg-Val-Val-Val-Arg-Val- |
| | Val-Arg-Val |
| 9 | RSRVVRSWSRV |
| | Arg-Ser-Arg-Val-Val-Arg-Ser-Trp-Ser-Arg-Val |
| 10 | RFVRRVRRFVRRVRRFVRRVRRFVRRVRRFVRRVRRFVRRVRRFVRRVR |
| | RFVRRVRRFVRRVRRFVRRVRRFVRRVRRFVRRVR |
| | Arg-Phe-Val-Arg-Arg-Val-Arg-Arg-Phe-Val-Arg-Arg-Val-Arg-Arg-Phe-Val- |
| | Arg-Arg-Val-Arg-Arg-Phe-Val-Arg-Arg-Val-Arg-Arg-Phe-Val-Arg-Arg-Val- |
| | Arg-Arg-Phe-Val-Arg-Arg-Val-Arg-Arg-Phe-Val-Arg-Arg-Val-Arg-Arg-Phe- |
| | Val-Arg-Arg-Val-Arg-Arg-Phe-Val-Arg-Arg-Val-Arg-Arg-Phe-Val-Arg-Arg- |
| | Val-Arg-Arg-Phe-Val-Arg-Arg-Val-Arg-Arg-Phe-Val-Arg-Arg-Val-Arg |
| 11 | RRTYSRSRRTYSRSRRTYSR |
| | Arg-Arg-Thr-Tyr-Ser-Arg-Ser-Arg-Arg-Thr-Tyr-Ser-Arg-Ser-Arg-Arg-Thr-Tyr- |
| | Ser-Arg |
| 12 | KVVSSIIEIISSVVKVVSSIIEIISSVV |
| | Lys-Val-Val-Ser-Ser-Ile-Ile-Glu-Ile-Ile-Ser-Ser-Val-Val-Lys-Val-Val-Ser-Ser- |
| | Ile-Ile-Glu-Ile-Ile-Ser-Ser-Val-Val |
| 13 | KKTHTKTKKTHTKTKKTHTK |
| | Lys-Lys-Thr-His-Thr-Lys-Thr-Lys-Lys-Thr-His-Thr-Lys-Thr-Lys-Lys-Thr-His- |
| | Thr-Lys |
| 14 | VVRVVRRVVRVVRRVVRVVRR |
| | Val-Val-Arg-Val-Val-Arg-Arg-Val-Val-Arg-Val-Val-Arg-Arg-Val-Val-Arg-Val- |
| | Val-Arg-Arg |
| 15 | RVVRVVRRVVRR |
| | Arg-Val-Val-Arg-Val-Val-Arg-Arg-Val-Val-Arg-Arg |
| 16 | RVVRVVRRWVRR |
| | Arg-Val-Val-Arg-Val-Val-Arg-Arg-Trp-Val-Arg-Arg |
| 17 | RWWRWWRRWWRR |
| | Arg-Trp-Trp-Arg-Trp-Trp-Arg-Arg-Trp-Trp-Arg-Arg |
| 18 | WRRWWRRWWRWWRRWWRR |
| | Trp-Arg-Arg-Trp-Trp-Arg-Arg-Trp-Trp-Arg-Trp-Trp-Arg-Arg-Trp-Trp-Arg-Arg |
| 19 | RRVVRRVRRVVRRVVRVVRRVVRR |
| | Arg-Arg-Val-Val-Arg-Arg-Val-Arg-Arg-Val-Val-Arg-Arg-Val-Val-Arg-Val- |
| | Val-Arg-Arg-Val-Val-Arg-Arg |
| 20 | RRWWRRWRRWWRRWWRWWRRWWRR |
| | Arg-Arg-Trp-Trp-Arg-Arg-Trp-Arg-Arg-Trp-Trp-Arg-Arg-Trp-Trp-Arg-Trp- |
| | Trp-Arg-Arg-Trp-Trp-Arg-Arg |
| 21 | VRRVVRRVVRVVRRVVRRVRRVVRRVVRVVRRVVRR |
| | Val-Arg-Arg-Val-Val-Arg-Arg-Val-Val-Arg-Val-Val-Arg-Arg-Val-Val-Arg- |
| | Arg-Val-Arg-Arg-Val-Val-Arg-Arg-Val-Val-Arg-Val-Val-Arg-Arg-Val-Val- |
| | Arg-Arg |
| 22 | VRRVWRRVVRVVRRWVRRVRRVWRRVVRVVRRWVRR |
| | Val-Arg-Arg-Val-Trp-Arg-Arg-Val-Val-Arg-Val-Val-Arg-Arg-Trp-Val-Arg- |
| | Arg-Val-Arg-Arg-Val-Trp-Arg-Arg-Val-Val-Arg-Val-Val-Arg-Arg-Trp-Val- |
| | Arg-Arg |
| 23 | RRVVRRVRRVVRRVVRVVRRVVRRVRRVVRRVVRVVRRVVRR |
| | Arg-Arg-Val-Val-Arg-Arg-Val-Arg-Arg-Val-Val-Arg-Arg-Val-Val-Arg-Val- |
| | Val-Arg-Arg-Val-Val-Arg-Arg-Val-Arg-Arg-Val-Val-Arg-Arg-Val-Val-Arg- |
| | Val-Val-Arg-Arg-Val-Val-Arg-Arg |
| 24 | RVVRVVRRVVRRVRRVVRRVVRVVRRVVRRVRRVVRRVVRVVRRVVR |
| | R |
| | Arg-Val-Val-Arg-Val-Val-Arg-Arg-Val-Val-Arg-Arg-Val-Arg-Arg-Val-Val- |
| | Arg-Arg-Val-Val-Arg-Val-Val-Arg-Arg-Val-Val-Arg-Arg-Val-Arg-Arg-Val- |
| | Val-Arg-Arg-Val-Val-Arg-Val-Val-Arg-Arg-Val-Val-Arg-Arg |
| 25 | RVVRVVRRWVRRVRRVWRRVVRVVRRWVRRVRRVWRRVVRVVRRW |
| | RVV |
| | Arg-Val-Val-Arg-Val-Val-Arg-Arg-Trp-Val-Arg-Arg-Val-Arg-Arg-Val-Trp- |
| | Arg-Arg-Val-Val-Arg-Val-Val-Arg-Arg-Trp-Val-Arg-Arg-Val-Arg-Arg-Val- |
| | Trp-Arg-Arg-Val-Val-Arg-Val-Val-Arg-Arg-Trp-Arg-Val-Val |

In some embodiments, the peptide or pharmaceutically acceptable salt thereof as described herein comprises SEQ ID NO: 1. In some embodiments, the peptide or pharmaceutically acceptable salt thereof as described herein comprises SEQ ID NO: 2. In some embodiments, the peptide or pharmaceutically acceptable salt thereof as described herein comprises SEQ ID NO: 3. In some embodiments, the peptide or pharmaceutically acceptable salt thereof as described herein comprises SEQ ID NO: 4. In some embodiments, the peptide or pharmaceutically acceptable salt thereof as described herein comprises SEQ ID NO: 5. In some embodiments, the peptide or pharmaceutically acceptable salt thereof as described herein comprises SEQ ID NO: 6. In some embodiments, the peptide or pharmaceutically acceptable salt thereof as described herein comprises SEQ ID NO: 7. In some embodiments, the peptide or pharmaceutically acceptable salt thereof as described herein comprises SEQ ID NO: 8. In some embodiments, the peptide or pharmaceutically acceptable salt thereof as described herein comprises SEQ ID NO: 9. In some embodiments, the peptide or pharmaceutically acceptable salt thereof as described herein comprises SEQ ID NO: 10. In some embodiments, the peptide or pharmaceutically acceptable salt thereof as described herein comprises SEQ ID NO: 11. In some embodiments, the peptide or pharmaceutically acceptable salt thereof as described herein comprises SEQ ID NO: 12. In some embodiments, the peptide or pharmaceutically acceptable salt thereof as described herein comprises SEQ ID NO: 13. In some embodiments, the peptide or pharmaceutically acceptable salt thereof as described herein comprises SEQ ID NO: 14. In some embodiments, the peptide or pharmaceutically acceptable salt thereof as described herein comprises SEQ ID NO: 15. In some embodiments, the peptide or pharmaceutically acceptable salt thereof as described herein comprises SEQ ID NO: 16. In some embodiments, the peptide or pharmaceutically acceptable salt thereof as described herein comprises SEQ ID NO: 17. In some embodiments, the peptide or pharmaceutically acceptable salt thereof as described herein comprises SEQ ID NO: 18. In some embodiments, the peptide or pharmaceutically acceptable salt thereof as described herein comprises SEQ ID NO: 19. In some embodiments, the peptide or pharmaceutically acceptable salt thereof as described herein comprises SEQ ID NO: 20. In some embodiments, the peptide or pharmaceutically acceptable salt thereof as described herein comprises SEQ ID NO: 21. In some embodiments, the peptide or pharmaceutically acceptable salt thereof as described herein comprises SEQ ID NO: 22. In some embodiments, the peptide or pharmaceutically acceptable salt thereof as described herein comprises SEQ ID NO: 23. In some embodiments, the peptide or pharmaceutically acceptable salt thereof as described herein comprises SEQ ID NO: 24. In some embodiments, the peptide or pharmaceutically acceptable salt thereof as described herein comprises SEQ ID NO: 25.

In some embodiments, the peptide or pharmaceutically acceptable salt thereof has at least 70% sequence identify to a polypeptide sequence of SEQ ID NO: 1, at least 70% sequence identify to a polypeptide sequence of SEQ ID NO: 2, at least 70% sequence identify to a polypeptide sequence of SEQ ID NO: 3, at least 70% sequence identify to a polypeptide sequence of SEQ ID NO: 4, at least 70% sequence identify to a polypeptide sequence of SEQ ID NO: 5, at least 70% sequence identify to a polypeptide sequence of SEQ ID NO: 6, at least 70% sequence identify to a polypeptide sequence of SEQ ID NO: 7, at least 70% sequence identify to a polypeptide sequence of SEQ ID NO: 8, at least 70% sequence identify to a polypeptide sequence of SEQ ID NO: 9, at least 70% sequence identify to a polypeptide sequence of SEQ ID NO: 10, at least 70% sequence identify to a polypeptide sequence of SEQ ID NO: 11, at least 70% sequence identify to a polypeptide sequence of SEQ ID NO: 12, at least 70% sequence identify to a polypeptide sequence of SEQ ID NO: 13, at least 70% sequence identify to a polypeptide sequence of SEQ ID NO: 14, at least 70% sequence identify to a polypeptide sequence of SEQ ID NO: 15, at least 70% sequence identify to a polypeptide sequence of SEQ ID NO: 16, at least 70% sequence identify to a polypeptide sequence of SEQ ID NO: 17, at least 70% sequence identify to a polypeptide sequence of SEQ ID NO: 18, at least 70% sequence identify to a polypeptide sequence of SEQ ID NO: 19, at least 70% sequence identify to a polypeptide sequence of SEQ ID NO: 20, at least 70% sequence identify to a polypeptide sequence of SEQ ID NO: 21, at least 70% sequence identify to a polypeptide sequence of SEQ ID NO: 22, at least 70% sequence identify to a polypeptide sequence of SEQ ID NO: 23, at least 70% sequence identify to a polypeptide sequence of SEQ ID NO: 24, or at least 70% sequence identify to a polypeptide sequence of SEQ ID NO: 25. In some embodiments, the peptide or pharmaceutically acceptable salt thereof has at least 70%, 75%, 80%, 85%, 90%, 95%, or 99% sequence identify to a polypeptide sequence listed in Table 1 and any increments of percentage therebetween.

In some embodiments, the pharmaceutical formulation comprises at least one peptide described herein as listed in Table 1. In some embodiments, the pharmaceutical formulation comprises one or more peptides described herein as listed in Table 1. In some embodiments, the pharmaceutical formulation comprises two or more peptides described herein as listed in Table 1. In some embodiments, the pharmaceutical formulation comprises three or more peptides described herein as listed in Table 1. In some embodiments, the pharmaceutical formulation comprises four or more peptides described herein as listed in Table 1. In some embodiments, the pharmaceutical formulation comprises five or more peptides described herein as listed in Table 1. In some embodiments, the pharmaceutical formulation comprises six or more peptides described herein as listed in Table 1. In some embodiments, the pharmaceutical formulation comprises seven or more peptides described herein as listed in Table 1. In some embodiments, the pharmaceutical formulation comprises eight or more peptides described herein as listed in Table 1. In some embodiments, the pharmaceutical formulation comprises nine or more peptides described herein as listed in Table 1. In some embodiments, the pharmaceutical formulation comprises ten or more peptides described herein as listed in Table 1. In some embodiments, the pharmaceutical formulation comprises eleven or more peptides described herein as listed in Table 1. In some embodiments, the pharmaceutical formulation comprises twelve or more peptides described herein as listed in Table 1. In some embodiments, the pharmaceutical formulation comprises thirteen or more peptides described herein as listed in Table 1. In some embodiments, the pharmaceutical formulation comprises fourteen or more peptides described herein as listed in Table 1. In some embodiments, the pharmaceutical formulation comprises fifteen or more peptides described herein as listed in Table 1.

A peptide disclosed herein can be a salt thereof. In some embodiments, recitation of the phrases “peptide” or “polypeptide” should be construed to include a salt thereof even if not explicitly recited. In some embodiments, a salt can include a carboxylate salt (e.g. formate, acetate, trifluoroacetate, trichloroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, pamoate, nicotinate, isonicotinate, cinnamate, oxalate, malonate, succinate, suberate, sebacate, fumarate, malate, maleate, hydroxymaleate, hippurate, phthalate or a terephthalate salts); a halide salt (e.g. chloride, bromide or iodide salts); a sulfonate salt (e.g. benzene sulfonate, methyl-, bromo- or chloro-benzenesulfonate, xylenesulfonate, methanesulfonate, trifluoromethanesulfonate, ethanesulfonate, propanesulfonate, hydroxyethanesulfonate, 1- or 2- naphthalene-sulfonate or 1,5-naphthalenedisulfonate salts); a sulfate salt; a pyrosulfate salt; a bisulfate salt; a sulfite salt; a bisulfite salt; a phosphate salt; a monohydrogenphosphate salt; a dihydrogenphosphate salt; a metaphosphate salt; a pyrophosphate salt; a nitrate salt; a chromium salt (e.g., octanoic acid); and the like.

In some embodiments, amino acids of the peptides described herein can be L-amino acids. In some embodiments, amino acids of the peptides described herein can be D-amino acids. In some embodiments, the peptides can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 D-amino acids and the rest are L-amino acids within the peptide sequence. In some embodiments, the peptides can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 L-amino acids and the rest are D-amino acids within the peptide sequence.

In some embodiments, a peptide can be formulated with one or more pharmaceutically acceptable salts. In some embodiments, a pharmaceutically acceptable salt can be a salt described in Berge et al, J. Pharm. Sci, 1977. In some embodiments, a pharmaceutically acceptable salts can include those salts prepared by reaction of a peptide with a mineral, organic acid or inorganic base, such salts including, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate and xylenesulfonate.

In some embodiments, a peptide can be formulated as a cleavable prodrug. The term “prodrug” as used herein, can refer to a drug precursor that, following administration to a subject and subsequent absorption, can be converted to an active, or a more active species via some process, such as conversion by a metabolic pathway. Thus, the term can encompass a derivative, which, upon administration to a recipient, can be capable of providing, either directly or indirectly, a peptide, pharmaceutically acceptable salt or a metabolite or residue thereof. Some prodrugs can have a chemical group present on a prodrug that renders it less active and/or confers solubility or some other property to the drug. Once the chemical group has been cleaved and/or modified from the prodrug the active drug can be generated. a prodrugs can be a prodrug that can increase the bioavailability of a peptide when administered to a subject (e.g. by allowing an administered peptide to be more readily absorbed) or which enhance delivery of the peptide to a biological compartment (e.g. the brain or lymphatic system).

### PH

In some embodiments, the pharmaceutical formulation described herein may further comprise a pH value of about 3.5 to about 5.5. The pharmaceutical formulation may comprise a pH value from 3.5 to 5.5, including increments therebetween, such as 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5, including increments therebetween. In some embodiments, the pharmaceutical formulation has a pH of about 3.5. In some embodiments, the pharmaceutical formulation has a pH of about 3.6. In some embodiments, the pharmaceutical formulation has a pH of about 3.7. In some embodiments, the pharmaceutical formulation has a pH of about 3.8. In some embodiments, the pharmaceutical formulation has a pH of about 3.9. In some embodiments, the pharmaceutical formulation has a pH of about 4.0. In some embodiments, the pharmaceutical formulation has a pH of about 4.1. In some embodiments, the pharmaceutical formulation has a pH of about 4.2. In some embodiments, the pharmaceutical formulation has a pH of about 4.3. In some embodiments, the pharmaceutical formulation has a pH of about 4.4. In some embodiments, the pharmaceutical formulation has a pH of about 4.5. In some embodiments, the pharmaceutical formulation has a pH of about 4.6. In some embodiments, the pharmaceutical formulation has a pH of about 4.7. In some embodiments, the pharmaceutical formulation has a pH of about 4.8. In some embodiments, the pharmaceutical formulation has a pH of about 4.9. In some embodiments, the pharmaceutical formulation has a pH of about 5.0. In some embodiments, the pharmaceutical formulation has a pH of about 5.1. In some embodiments, the pharmaceutical formulation has a pH of about 5.2. In some embodiments, the pharmaceutical formulation has a pH of about 5.3. In some embodiments, the pharmaceutical formulation has a pH of about 5.4. In some embodiments, the pharmaceutical formulation has a pH of about 5.5.

In some embodiments, the pharmaceutical formulation comprising a peptide or pharmaceutically acceptable salt is at a pH value of at least 3.5 to at least 5.5, at least 3.6 to at least 5.5, at least 3.7 to at least 5.5, at least 3.8 to at least 5.5, at least 3.9 to at least 5.5, at least 4.0 to at least 5.5, at least 4.1 to at least 5.5, at least 4.2 to at least 5.5, at least 4.3 to at least 5.5, at least 4.4 to at least 5.5, at least 4.5 to at least 5.5, at least 4.6 to at least 5.5, at least 4.7 to at least 5.5, at least 4.8 to at least 5.5, at least 4.9 to at least 5.5, at least 5.0 to at least 5.5, at least 5.1 to at least 5.5, at least 5.2 to at least 5.5, at least 5.3 to at least 5.5, or at least 5.4 to at least 5.5. In some embodiments, the pharmaceutical formulation comprising a peptide or pharmaceutically acceptable salt is at a pH value of at least 3.5 to at least 5.5, at least 3.5 to at least 5.4, at least 3.5 to at least 5.3, at least 3.5 to at least 5.2, at least 3.5 to at least 5.1, at least 3.5 to at least 5.0, at least 3.5 to at least 4.9, at least 3.5 to at least 4.8, at least 3.5 to at least 4.7, at least 3.5 to at least 4.6, at least 3.5 to at least 4.5, at least 3.5 to at least 4.4, at least 3.5 to at least 4.3, at least 3.5 to at least 4.2, at least 3.5 to at least 4.1, at least 3.5 to at least 4.0, at least 3.5 to at least 3.9, at least 3.5 to at least 3.8, at least 3.5 to at least 3.7, or at least 3.5 to at least 3.6. In some embodiments, the pharmaceutical formulation comprising a peptide or pharmaceutically acceptable salt is at a pH value of at least 3.5 to at least 5.5, at least 3.6 to at least 5.4, at least 3.7 to at least 5.3, at least 3.8 to at least 5.2, at least 3.9 to at least 5.1, at least 4.0 to at least 5.0, at least 4.1 to at least 4.9, at least 4.2 to at least 4.8, at least 4.3 to at least 4.7, at least 4.4 to at least 4.6, at least 4.0 to about 5.5, at least 4.5 to about 5.5, at least 5.0.

In some embodiments, the pharmaceutical formulation comprising a peptide or pharmaceutically acceptable salt is at a pH value of at most 3.5 to at most 5.5, at most 3.6 to at most 5.5, at most 3.7 to at most 5.5, at most 3.8 to at most 5.5, at most 3.9 to at most 5.5, at most 4.0 to at most 5.5, at most 4.1 to at most 5.5, at most 4.2 to at most 5.5, at most 4.3 to at most 5.5, at most 4.4 to at most 5.5, at most 4.5 to at most 5.5, at most 4.6 to at most 5.5, at most 4.7 to at most 5.5, at most 4.8 to at most 5.5, at most 4.9 to at most 5.5, at most 5.0 to at most 5.5, at most 5.1 to at most 5.5, at most 5.2 to at most 5.5, at most 5.3 to at most 5.5, or at most 5.4 to at most 5.5. In some embodiments, the pharmaceutical formulation comprising a peptide or pharmaceutically acceptable salt is at a pH value of at most 3.5 to at most 5.5, at most 3.5 to at most 5.4, at most 3.5 to at most 5.3, at most 3.5 to at most 5.2, at most 3.5 to at most 5.1, at most 3.5 to at most 5.0, at most 3.5 to at most 4.9, at most 3.5 to at most 4.8, at most 3.5 to at most 4.7, at most 3.5 to at most 4.6, at most 3.5 to at most 4.5, at most 3.5 to at most 4.4, at most 3.5 to at most 4.3, at most 3.5 to at most 4.2, at most 3.5 to at most 4.1, at most 3.5 to at most 4.0, at most 3.5 to at most 3.9, at most 3.5 to at most 3.8, at most 3.5 to at most 3.7, or at most 3.5 to at most 3.6. In some embodiments, the pharmaceutical formulation comprising a peptide or pharmaceutically acceptable salt is at a pH value of at most 3.5 to at most 5.5, at most 3.6 to at most 5.4, at most 3.7 to at most 5.3, at most 3.8 to at most 5.2, at most 3.9 to at most 5.1, at most 4.0 to at most 5.0, at most 4.1 to at most 4.9, at most 4.2 to at most 4.8, at most 4.3 to at most 4.7, at most 4.4 to at most 4.6, at most 4.0 to about 5.5, at most 4.5 to about 5.5, at most 5.0.

In some embodiments, the pharmaceutical formulation may further comprise a pH adjusting agent, such as hydrochloric acid, sodium hydroxide, ammonium hydroxide, other pH adjusting agents known to those skilled in the art, or combinations thereof to the aqueous carrier. In some embodiments, the pH adjusting agent is hydrochloric acid. In some embodiments, the pH adjusting agent is sodium hydroxide. In some embodiments, the pH adjusting agent is ammonium hydroxide. In some embodiments, the pH adjusting agent is hydrochloric acid, sodium hydroxide, or any combination thereof.

In some embodiments, the pharmaceutical formulation further comprises a pH buffer or pH buffering agent. Non-limiting examples of suitable pH buffers or pH buffering agents includes sodium citrate, citric acid, sodium acetate, acetic acid, phosphoric acid, trisodium phosphate, lactic acid, sodium lactate, tartaric acid, monosodium tartrate, sodium tartrate dibasic, 4-(2-hydroxyethyl)-1-piperazineethanesulfonic acid (HEPES), piperazine-N,N′-bis(2-ethanesulfonic acid) (PIPES), 2-(N-morpholino)ethanesulfonic acid (MES), other pH buffers known to those skilled in the art, or combinations thereof. In some embodiments, the pH buffer or pH buffering agent comprises sodium citrate. In some embodiments, the pH buffer or pH buffering agent comprises citric acid. some embodiments, the pH buffer or pH buffering agent comprises sodium acetate. In some embodiments, the pH buffer or pH buffering agent comprises acetic acid. In some embodiments, the pH buffer or pH buffering agent comprises phosphoric acid. In some embodiments, the pH buffer or pH buffering agent comprises trisodium phosphate. In some embodiments, the pH buffer or pH buffering agent comprises lactic acid. In some embodiments, the pH buffer or pH buffering agent comprises sodium lactate. In some embodiments, the pH buffer or pH buffering agent comprises tartaric acid. In some embodiments, the pH buffer or pH buffering agent comprises monosodium tartrate. In some embodiments, the pH buffer or pH buffering agent comprises sodium tartrate dibasic. In some embodiments, the pH buffer or pH buffering agent comprises 4-(2-hydroxyethyl)-1-piperazineethanesulfonic acid (HEPES). In some embodiments, the pH buffer or pH buffering agent comprises piperazine-N,N′-bis(2-ethanesulfonic acid) (PIPES). In some embodiments, the pH buffer or pH buffering agent comprises 2-(N-morpholino)ethanesulfonic acid (MES). In some embodiments, the pH buffer or pH buffering agent comprises sodium hydrogen phosphate, sodium dihydrogen phosphate, potassium dihydrogen phosphate, potassium hydrogen phosphate, glycine, tris(hydroxymethyl)aminomethane, and any combination thereof. In some embodiments, the pH buffer or pH buffering agent comprises sodium hydrogen phosphate. In some embodiments, the pH buffer or pH buffering agent comprises sodium dihydrogen phosphate. In some embodiments, the pH buffer or pH buffering agent comprises potassium dihydrogen phosphate. In some embodiments, the pH buffer or pH buffering agent comprises potassium hydrogen phosphate. In some embodiments, the pH buffer or pH buffering agent comprises glycine. In some embodiments, the pH buffer or pH buffering agent comprises tris(hydroxymethyl)aminomethane. In some embodiments, the pH buffer comprises a phosphate buffer. In some embodiments, the phosphate buffer comprises Dulbecco's phosphate buffered saline (dPBS).

In some embodiments, the pharmaceutical formulation can be free of a pH buffering agent or pH buffer.

In some embodiments, the peptide or pharmaceutically acceptable salt thereof can have an increase in solubility. In some embodiments, the solubility of the peptide or pharmaceutically acceptable salt thereof is a function of the pH of the pharmaceutical formulation. In some embodiments, the solubility of the peptide or pharmaceutically acceptable salt thereof increases as the pH value of a pharmaceutical formulation is lowered. In some embodiments, the solubility of the peptide or pharmaceutically acceptable salt thereof in a pharmaceutical formulation increases where the pH of the pharmaceutical formulation is below about 7, about 7.1, about 7.2, about 7.3, about 7.4, about 7.5, about 7.6, about 7.7, about 7.8, about 7.9, about 8, about 8.1, about 8.2, about 8.3, about 8.4, or about 8.5. In some embodiments, the solubility of the peptide or pharmaceutically acceptable salt thereof in a pharmaceutical formulation increases where the pH of the pharmaceutical formulation is about 3.5 to about 6.9, about 3.5 to about 5.5, about 3.5 to about 5.0, about 3.5 to about 6.0, about 3.5 to about 6.5. In some embodiments, the solubility of the peptide or pharmaceutically acceptable salt thereof in a pharmaceutical formulation increases with a pH of about 3.5, about 3.6, about 3.7, about 3.8, about 3.9, about 4, about 4.1, about 4.2, about 4.3, about 4.4, about 4.5, about 4.6, about 4.7, about 4.8, about 4.9 about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7 about 6.8, or about 6.9 in the pharmaceutical formulation. In some embodiments, the solubility of the peptide or pharmaceutically acceptable salt thereof in a pharmaceutical formulation increases with a pH of the pharmaceutical formulation of about 5. In some embodiments, the solubility of the peptide or pharmaceutically acceptable salt thereof in a pharmaceutical formulation increases with a pH of about 5 compared to a pH above 5 in a pharmaceutical formulation as disclosed herein.

### Stability

In some exemplary embodiments, a pharmaceutical formulation can be stable for at least about 1 month, 2 months, 3 months, 4 months, 5 months, 6 months, 7 months, 8 months, 9 months, 10 months, 11 months, 1 year, 2 years, 3 years, 4 years, or 5 years when stored in a closed container at 0%, 5%, 10%, 15%, 20%, 25%, 30%, 35%, 40%, 45%, 50%, 55%, 60%, 65%, 70%, 75%, 80%, 85%, 90%, or 95% relative humidity at a temperature of from about −80° C. to about 70° C., from about −80° C. to about 60° C., from about −70° C. to about 60° C., from about −50° C. to about 60° C., from about −40° C. to about 60° C., from about −30° C. to about 60° C., from about −20° C. to about 60° C., from about −10° C. to about 60° C., from about 0° C. to about 60° C., from about 5° C. to about 60, from about 10° C. to about 60° C., from about 20° C. to about 60° C., from about 25° C. to about 60° C., from about 30° C. to about 60° C., from about −80° C. to about 50° C., from about −80° C. to about 40° C., from about −80° C. to about 30° C., from about −80° C. to about 20° C., from about −80° C. to about 10° C., from about −80° C. to about 5° C., from about −80° C. to about 0° C., from about −80° C. to about −10° C., from about −80° C. to about −20° C., from about −80° C. to about −30° C., from about −20° C. to about 40° C., about −20° C., about 5° C., about 40° C., or room temperature and the pH of the pharmaceutical formulation is between about 3.5 to about 5.5, about 4.0 to about 5.5, about 4.1 to about 5.5, about 4.2 to about 5.5, about 4.3 to about 5.5, about 4.4 to about 5.5, about 4.5 to about 5.5, about 4.6 to about 5.5, about 4.7 to about 5.5, about 4.8 to about 5.5, about 4.9 to about 5.5, about 5.0 to about 5.5, about 5.1 to about 5.5, about 5.2 to about 5.5, about 5.3 to about 5.5, about 5.4 to about 5.5, about 4.5 to about 5.4, about 4.6 to about 5.3, about 4.7 to about 5.3, about 4.8 to about 5.2, about 4.9 to about 5.1, or about 5.0. In some embodiments, room temperature may be defined as about 20° C. to about 22° C. Stability can be determined by determining an amount of peptide remaining after a period of time. In some embodiments, at least about 10%, 20%, 30%, 40%, 50%, 60%, 70%, 80%, 90%, 91%, 92%, 93%, 94%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4% , 99.5%, 99.6% , 99.7%, 99.8%, 99.9%, or 100% remains after a time period.

Stability can further be determined by determining an amount of peptide and certain impurities remaining impurities after a period of time. In some embodiments, the amount of impurities after a period of time may be at most 6%, at most 5.9%, at most 5.8%, at most 5.7%, at most 5.6%, at most 5.5%, at most 5.4%, at most 5.3%, at most 5.2%, at most 5.1%, at most 5%, at most 4.9%, at most 4.8%, at most 4.7%, at most 4.6%, at most 4.5%, at most 4.4%, at most 4.3%, at most 4.2%, at most 4.1%, at most 4.0%, at most 3.9%, at most 3.8%, at most 3.7%, at most 3.6%, at most 3.6%, at most 3.5%, at most 3.4%, at most 3.3%, at most 3.2%, at most 3.1%, at most 3%, at most 2.9%, at most 2.8%, at most 2.7%, at most 2.6%, at most 2.5%, at most 2.4%, at most 2.3%, at most 2.2%, at most 2.1%, at most 2%, at most 1.9%, at most 1.8%, at most 1.7%, at most 1.6%, at most 1.5%, at most 1.4%, at most 1.3%, at most 1.2%, at most 1.1%, at most 1%, at most 0.9%, at most 0.8%, most 0.7%, at most 0.6%, at most 0.5%, at most 0.4%, at most 0.4%, at most 0.3%, at most 0.2%, or at most 0.1%.

In some embodiments, the amount of impurities after period of time may be about 6%, about 5.9%, about 5.8%, about 5.7%, about 5.6%, about 5.5%, about 5.4%, about 5.3%, about 5.2%, about 5.1%, about 5%, about 4.9%, about 4.8%, about 4.7%, about 4.6%, about 4.5%, about 4.4%, about 4.3%, about 4.2%, about 4.1%, about 4.0%, about 3.9%, about 3.8%, about 3.7%, about 3.6%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, most 0.7%, about 0.6%, about 0.5%, about 0.4%, about 0.4%, about 0.3%, about 0.2%, or about 0.1%.

In some embodiments, the amount of peptide and/or impurities are measured after storage of a period of 1 day, 2 days, 3 days, 4 days, 5 days, 6 days, 7 days, 10 days, 14 days, 3 weeks, 1 month, 54 days, 2 months, 3 months, 4, months, 5 months, 6 months, 7 months, 8 months, 9 months, 10 months, 11 months, 1 year, 2 years, 3 years, 4 years or 5 years. In some embodiments, the amount of peptide and/or impurities are measured after storage of a period of 7 days. In some embodiments, the amount of peptide and/or impurities are measured after storage of a period of 14 days. In some embodiments, the amount of peptide and/or impurities are measured after storage of a period of 1 month. In some embodiments, the amount of peptide and/or impurities are measured after storage of a period of 2 months. In some embodiments, the amount of peptide and/or impurities are measured after storage of a period of 54 days. In some embodiments, the amount of peptide and/or impurities are measured after storage of a period of 3 months. In some embodiments, the amount of peptide and/or impurities are measured after storage of a period of 4 months. In some embodiments, the amount of peptide and/or impurities are measured after storage of a period of 5 months. In some embodiments, the amount of peptide and/or impurities are measured after storage of a period of 6 months. In some embodiments, the amount of peptide and/or impurities are measured after storage of a period of 7 months. In some embodiments, the amount of peptide and/or impurities are measured after storage of a period of 8 months. In some embodiments, the amount of peptide and/or impurities are measured after storage of a period of 9 months. In some embodiments, the amount of peptide and/or impurities are measured after storage of a period of 10 months. In some embodiments, the amount of peptide and/or impurities are measured after storage of a period of 11 months. In some embodiments, the amount of peptide and/or impurities are measured after storage of a period of 1 year. In some embodiments, the amount of peptide and/or impurities are measured after storage of a period of 2 years. In some embodiments, the amount of peptide and/or impurities are measured after storage of a period of 3 years.

In some embodiments, the amount of peptide and/or impurities remaining after a period of time can be determined by performing an area under the curve analysis of a mass spectra. In some embodiments, the amount of peptide and/or impurities remaining after a period of time can be determined by performing an area under the curve analysis of a mass spectra from tandem mass spectrometry (MS/MS). In some embodiments, the amount of peptide and/or impurities remaining after a period of time can be determined by using a UV-Vis Assay. In some embodiments, the amount of peptide and/or impurities remaining after a period of time can be determined by performing an area under the curve analysis of a High-performance liquid chromatography (HPLC). In some embodiments, the amount of peptide and/or impurities remaining after a period of time can be determined by performing an area under the curve analysis of an Ultra-performance liquid chromatography (UPLC). In some embodiments, the amount of peptide remaining after a period of time can be measured as purity by % or purity by % area under the curve. In some embodiments, the amount of impurities remaining after a period of time can measured as % total impurities or % total impurities area under the curve.

In some embodiments, the pharmaceutical formulation comprises impurities at a measured weight % of the formulation after a period of time. In some embodiments, the pharmaceutical formulation comprises at most 5% by weight of a plurality of impurities after a period of time. In some embodiments, the pharmaceutical formulation comprises at most 4.5% by weight of a plurality of impurities as measured by HPLC after a period of time. In some embodiments, the pharmaceutical formulation comprises at most 4% by weight of a plurality of impurities as measured by HPLC after a period of time. In some embodiments, the pharmaceutical formulation comprises at most 3.5% by weight of a plurality of impurities as measured by HPLC after a period of time. In some embodiments, the pharmaceutical formulation comprises at most 3% by weight of a plurality of impurities as measured by HPLC after a period of time. In some embodiments, the pharmaceutical formulation comprises at most 2.5% by weight of a plurality of impurities as measured by HPLC after a period of time. In some embodiments, the pharmaceutical formulation comprises at most 2% by weight of a plurality of impurities as measured by HPLC after a period of time. In some embodiments, the pharmaceutical formulation comprises at most 1.5% by weight of a plurality of impurities as measured by HPLC after a period of time. In some embodiments, the pharmaceutical formulation comprises at most 1% by weight of a plurality of impurities as measured by HPLC after a period of time. In some embodiments, the pharmaceutical formulation comprises at most 0.5% by weight of a plurality of impurities as measured by HPLC after a period of time. In some embodiments, the pharmaceutical formulation comprises at most 0.1% by weight of a plurality of impurities as measured by HPLC after a period of time.

In some embodiments, the pharmaceutical formulation comprises impurities at a measured weight % of the formulation after a period of time. In some embodiments, the pharmaceutical formulation comprises at most 5% by weight of a plurality of impurities after a period of time. In some embodiments, the pharmaceutical formulation comprises at most 4.5% by weight of a plurality of impurities as measured by UPLC after a period of time. In some embodiments, the pharmaceutical formulation comprises at most 4% by weight of a plurality of impurities as measured by UPLC after a period of time. In some embodiments, the pharmaceutical formulation comprises at most 3.5% by weight of a plurality of impurities as measured by UPLC after a period of time. In some embodiments, the pharmaceutical formulation comprises at most 3% by weight of a plurality of impurities as measured by UPLC after a period of time. In some embodiments, the pharmaceutical formulation comprises at most 2.5% by weight of a plurality of impurities as measured by UPLC after a period of time. In some embodiments, the pharmaceutical formulation comprises at most 2% by weight of a plurality of impurities as measured by UPLC after a period of time. In some embodiments, the pharmaceutical formulation comprises at most 1.5% by weight of a plurality of impurities as measured by UPLC after a period of time. In some embodiments, the pharmaceutical formulation comprises at most 1% by weight of a plurality of impurities as measured by UPLC after a period of time. In some embodiments, the pharmaceutical formulation comprises at most 0.5% by weight of a plurality of impurities as measured by UPLC after a period of time. In some embodiments, the pharmaceutical formulation comprises at most 0.1% by weight of a plurality of impurities as measured by UPLC after a period of time.

In some embodiments, the pharmaceutical formulation comprises at most 5% by weight of a plurality of impurities as measured by UV-Vis spectroscopy after a period of time. In some embodiments, the pharmaceutical formulation comprises at most 4.5% by weight of a plurality of impurities as measured by UV-Vis spectroscopy after a period of time. In some embodiments, the pharmaceutical formulation comprises at most 4% by weight of a plurality of impurities as measured by UV-Vis spectroscopy after a period of time. In some embodiments, the pharmaceutical formulation comprises at most 3.5% by weight of a plurality of impurities as measured by UV-Vis spectroscopy after a period of time. In some embodiments, the pharmaceutical formulation comprises at most 3% by weight of a plurality of impurities as measured by UV-Vis spectroscopy after a period of time. In some embodiments, the pharmaceutical formulation comprises at most 2.5% by weight of a plurality of impurities as measured by UV-Vis spectroscopy after a period of time. In some embodiments, the pharmaceutical formulation comprises at most 2% by weight of a plurality of impurities as measured by UV-Vis spectroscopy after a period of time. In some embodiments, the pharmaceutical formulation comprises at most 1.5% by weight of a plurality of impurities as measured by UV-Vis spectroscopy after a period of time. In some embodiments, the pharmaceutical formulation comprises at most 1% by weight of a plurality of impurities as measured by UV-Vis spectroscopy after a period of time. In some embodiments, the pharmaceutical formulation comprises at most 0.5% by weight of a plurality of impurities as measured by UV-Vis spectroscopy after a period of time. In some embodiments, the pharmaceutical formulation comprises at most 0.1% by weight of a plurality of impurities as measured by UV-Vis spectroscopy after a period of time.

In some embodiments, the pharmaceutical formulation, peptide, or pharmaceutically acceptable salt thereof can be stored at from about 0.001 μg/mL to about 10 g/mL. In some embodiments, the pharmaceutical formulation, peptide, or pharmaceutically acceptable salt thereof can be stored at about 0.001 μg/mL, about 0.01 μg/mL, about 0.1 μg/mL, about 1 μg/mL, about 10 μg/mL, about 100 μg/mL, about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 200 mg/mL, about 220 mg/mL, about 240 mg/mL, about 260 mg/mL, about 280 mg/mL, about 300 mg/mL, about 320 mg/mL, about 340 mg/mL, about 360 mg/mL, about 380 mg/mL, about 400 mg/mL, about 440 mg/mL, about 460 mg/mL, about 480 mg/mL, 500 mg/mL, about 1 g/mL, or about 10 g/mL. In some embodiments, the pharmaceutical formulation, peptide, or pharmaceutically acceptable salt thereof can be stored at about 15 mg/mL, about 30 mg/mL, about 40 mg/mL, about 50 mg/mL, about 60 mg/mL, about 70 mg/mL, about 80 mg/mL, about 90 mg/mL, or about 40 mg/mL. In some embodiments, the pharmaceutical formulation, peptide, or pharmaceutically acceptable salt thereof can be stored at about 40 mg/mL. In some embodiments, the pharmaceutical formulation, peptide, or pharmaceutically acceptable salt thereof can be stored at about 80 mg/mL. In some embodiments, the pharmaceutical formulation, peptide, or pharmaceutically acceptable salt thereof can be stored at about 90 mg/mL. In some embodiments, the pharmaceutical formulation, peptide, or pharmaceutically acceptable salt thereof can be stored at about 100 mg/mL. In some embodiments, the pharmaceutical formulation, peptide, or pharmaceutically acceptable salt thereof can be stored at about 200 mg/mL. In some embodiments, the pharmaceutical formulation, peptide, or pharmaceutically acceptable salt thereof can be stored at about 300 mg/mL. In some embodiments, the pharmaceutical formulation, peptide, or pharmaceutically acceptable salt thereof can be stored at about 400 mg/mL. In some embodiments, the pharmaceutical formulation, peptide, or pharmaceutically acceptable salt thereof can be stored at about 500 mg/mL. In some embodiments, the pharmaceutical formulation, peptide, or pharmaceutically acceptable salt thereof can be stored at about 1 g/mL. In some embodiments, the pharmaceutical formulation, peptide, or pharmaceutically acceptable salt thereof can be stored at about 10 g/mL.

In some embodiments, the pharmaceutical formulation may be hypertonic, isotonic, or hypotonic. In some embodiments, the pharmaceutical formulation may be hypertonic. In some embodiments, the pharmaceutical formulation may be isotonic. In some embodiments, the pharmaceutical formulation may be hypotonic.

In some embodiments, the pharmaceutical formulation, peptide, or pharmaceutically acceptable salt thereof can be stored as a liquid or solid. In some embodiments, the pharmaceutical formulation, peptide, or pharmaceutically acceptable salt thereof can be stored as a liquid. In some embodiments, the pharmaceutical formulation, peptide, or pharmaceutically acceptable salt thereof can be stored as a solid.

Without being bound by a certain theory, an impurity or a plurality of impurities may arise from acid-catalyzed C-terminal deamidation that converts the C-terminal —CONH₂ (amide) to —CO₂H (carboxylic acid). Without being bound by a certain theory, high basicity of a peptide sequence or amino acid may contribute to long-term stability. Without being bound by a certain theory, high level of acid present in a peptide formulation may contribute to long-term stability. Without being bound by a certain theory, an acidic peptide formulation may lead to long-term stability. Without being bound by a certain theory, a peptide formulation with a pH of at least less than 7 may lead to long-term stability. Without being bound by a certain theory, a peptide formulation with a pH of about 5 may lead to long-term stability. Without being bound by a certain theory, an impurity or a plurality of impurities may arise from deamination of at least one Arg, at least one Arg converts into citrulline, loss of at least one amino acid, loss of at least one side chain, or loss or cleavage of at least one Arg side chain.

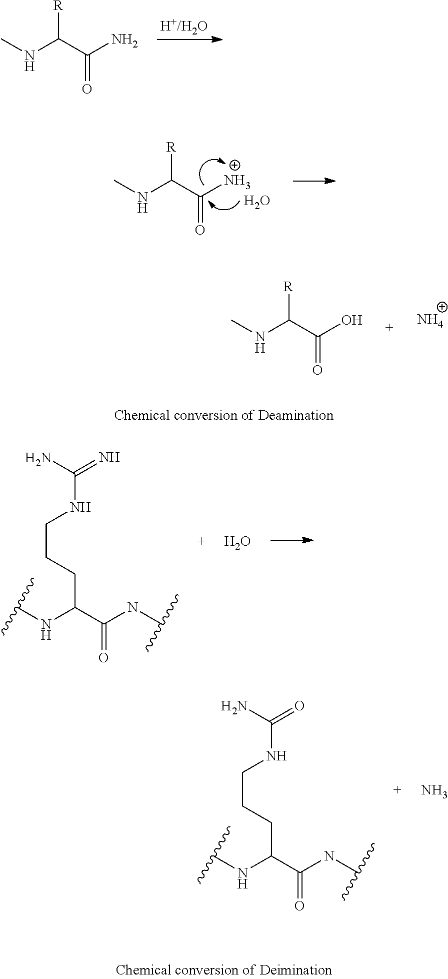

### Pharmaceutically Acceptable Excipients

At least one peptide disclosed herein can be formulated as a pharmaceutical formulation. In some embodiments, a pharmaceutical formulation can comprise a peptide described herein and at least one excipient. By “pharmaceutically acceptable”, it is meant that the carrier, diluent, or excipient must be compatible with the other ingredients of the formulation and not deleterious to the recipient thereof. The term “compatible”, as used herein, means that the components of the formulation are capable of being commingled with the subject compound, and with each other, in a manner such that there is no interaction that would substantially reduce the pharmaceutical efficacy of the formulation under ordinary use situations.

In some embodiments, a pharmaceutical formulation can comprise an excipient. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, and/or a coloring agent. In some embodiments, the pharmaceutical formulation further comprises one or more additional pharmaceutically acceptable excipients. See, e.g., Remington: The Science and Practice of Pharmacy (Gennaro, 21^{st} Ed. Mack Pub. Co., Easton, Pa. (2005) for a list of pharmaceutically acceptable excipients. In some embodiments, the pharmaceutically acceptable excipient is of sufficiently high purity and sufficiently low toxicity to render them suitable for administration preferably to an animal, preferably a mammal, being treated.

In some embodiments, an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments, an excipient can comprise a binder. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof. The binders that can be used in a pharmaceutical formulation can be selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, niannitol and water or a combination thereof.

In some embodiments, an excipient can comprise a lubricant. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminium stearate), fatly acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments, an excipient can comprise a dispersion enhancer. Non-limiting examples of suitable dispersants can include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, an excipient can comprise a disintegrant. In some embodiments a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant can be an effervescent disintegrant. Non-limiting examples of suitable effervescent disintegrants can include sodium bicarbonate in combination with citric acid, and sodium bicarbonate in combination with tartaric acid.

In some embodiments, an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. In some embodiments, the excipient may be a sugar. Non-limiting examples of suitable sugars can include glucose, sucrose, dextrose, lactose, maltodextrin, fructose, and mixtures thereof.

In some embodiments, an excipient can comprise a coloring agent. Non-limiting examples of suitable color agents can include food, drug and cosmetic colors (FD&C), drug and cosmetic colors (D&C), and external drug and cosmetic colors (Ext. D&C). A coloring agent can be used as dyes.

In some embodiments, an excipient can comprise an isotonicity agent. Examples can include, but are not limited to: sodium chloride, calcium chloride, potassium chloride, sodium lactate, copper chloride, copper sulfate, monopotassium phosphate, sucrose, dextrose, or glucose. In some embodiments, the isotonicity agent is sodium chloride.

In some embodiments, an excipient can comprise a chelator. In some embodiments, a chelator can be a fungicidal chelator. Examples can include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11] pentatriacontane hexahydrobromide; or triethylenetetramine-N,N,N′,N″,N′″,N′″-hexaacetic acid.

In some embodiments, an excipient can comprise a diluent. Non-limiting examples of diluents can include water, glycerol, methanol, ethanol, and other similar biocompatible diluents. In some embodiments, a diluent can be an aqueous acid such as acetic acid, citric acid, maleic acid, hydrochloric acid, phosphoric acid, nitric acid, sulfuric acid, or similar. In other cases, a diluent can be selected from a group comprising alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, caoline, lactose, maltose, mannitol, simethicone, sorbitol, starch, pregelatinized starch, talc, xylitol and/or anhydrates, hydrates and/or pharmaceutically acceptable derivatives thereof or combinations thereof.

In other embodiments, an excipient can comprise a surfactant. Surfactants can be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

In some embodiments, an excipient can comprise an aqueous carrier. In some embodiments, the aqueous carrier is lactated Ringer's solution, normal saline (0.9% w/v), sterile water for injection, or aqueous sodium carbonate. In some embodiments, the aqueous carrier is lactated Ringer's solution. In some embodiments, the aqueous carrier is normal saline (0.9% w/v). In some embodiments, the aqueous carrier is sterile water for injection. In some embodiments, the aqueous carrier is aqueous sodium bicarbonate. In some embodiments, the aqueous sodium bicarbonate is 8.4% sodium bicarbonate. In some embodiments, the aqueous carrier is physiologically isotonic, physiologically hypotonic, or physiologically hypertonic. In some embodiments, the aqueous carrier is physiologically isotonic. In some embodiments, the aqueous carrier is physiologically hypotonic. In some embodiments, the aqueous carrier is physiologically hypotonic (sub-physiologic osmolarity or osmolality), for example, modified versions of lactated Ringer's solution, normal saline (0.9% w/v), or aqueous sodium bicarbonate diluted with water. In some embodiments, the aqueous carrier is physiologically hypertonic. In some embodiments, the aqueous carrier has a total osmolarity ranging from about 1 milliosmoles per one liter (mOsm/L) to about 5,000 mOsm/L. In some embodiments, the aqueous carrier has a total osmolarity of about 1 mOsm/L, about 50 mOsm/L, about 100 mOsm/L, about 150 mOsm/L, about 200 mOsm/L, about 250 mOsm/L, about 300 mOsm/L, about 350 mOsm/L, about 400 mOsm/L, about 450 mOsm/L, about 500 mOsm/L, about 1000 mOsm/L, about 1500 mOsm/L, about 2000 mOsm/L, about 2500 mOsm/L, about 3000 mOsm/L, about 3500 mOsm/L, about 4000 mOsm/L, about 4500 mOsm/L, or about 5000 mOsm/L. In some embodiment, the aqueous carrier has a total osmolality ranging from about 1 milliosmole per kilogram (mOsm/kg) from 5000 mOsm/kg. In some embodiments, the aqueous carrier has a total osmolarity of about 1 mOsm/kg, about 50 mOsm/kg, about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, about 500 mOsm/kg, about 1000 mOsm/kg, about 1500 mOsm/kg, about 2000 mOsm/kg, about 2500 mOsm/kg, about 3000 mOsm/kg, about 3500 mOsm/kg, about 4000 mOsm/kg, about 4500 mOsm/kg, or about 5000 mOsm/kg. In some embodiments, the aqueous carrier may have a total ionic strength ranging from about 0.001 molar (M) and 1.0 M. In some embodiments, the aqueous carrier may have a total ionic strength of about 0.001 M, about 0.01 M, about 0.015 M, about 0.02 M, about 0.025 M, about 0.03 M, about 0.035 M, about 0.04 M, about 0.05 M, about 0.055 M, about 0.06 M, about 0.065 M, about 0.07 M, about 0.075 M, about 0.08 M, about 0.085 M, about 0.09 M, about 0.1 M, about 0.12 M, about 0.14 M, about 0.15 M, about 0.16 M, about 0.18 M, about 0.2 M, about 0.22 M, about 0.24 M, about 0.25 M, about 0.26 M, about 0.28 M, about 0.03 M, about 0.35 M, about 0.4 M, about 0.45 M, about 0.5 M, about 0.55 M, about 0.6 M, about 0.65 M, about 0.7 M, about 0.75 M about 0.8 M, about 0.85 M, about 0.9 M, about 0.95 M, or about 1.0 M. In some embodiments, the aqueous carrier may have an electrolyte concentration ranging from about 0.1 milliequivalent per mL (mEq/mL) to about 1000 mEq/mL. In some embodiments, the aqueous carrier may have an electrolyte concentration of about 0.1 mEq/mL, about 1 mEq/mL, about 25 mEq/mL, about 50 mEq/mL, about 100 mEq/mL, about 150 mEq/mL, about 200 mEq/mL, about 250 mEq/mL, about 300 mEq/mL, about 350 mEq/mL, about 400 mEq/mL, about 450 mEq/mL, about 500 mEq/mL, about 550 mEq/mL, about 600 mEq/mL, about 650 mEq/mL, about 700 mEq/mL, about 750 mEq/mL, about 800 mEq/mL, about 850 mEq/mL, about 900 mEq/mL, about 950 mEq/mL, or about 1000 mEq/mL. In some embodiments, the aqueous carrier may have an electrolyte concentration of about 1 mEq/mL to about 500 mEq/mL, about 50 mEq/mL to about 500 mEq/mL, about 100 mEq/mL to about 300 mEq/mL, about 125 mEq/mL to about 250 mEq/mL, about 100 mEq/mL to about 500 mEq/mL, or about 100 mEq/mL to about 1000 mEq/mL.

In some embodiments, the pharmaceutical formulation is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof. A weight fraction of an excipient or combination of excipients in a pharmaceutical formulation can be less than about 80%, 70%, 60%, 50%, 45%, 40%, 35%, 30%, 25%, 20%, 15%, 10%, 5%, 4%, 3%, 2%, or 1% as compared to a total weight of a pharmaceutical formulation. See, e.g., Remington: The Science and Practice of Pharmacy (Gennaro, 21^{st} Ed. Mack Pub. Co., Easton, Pa. (2005).

### METHODS OF TREATMENT

### Administration

A pharmaceutical formulation disclosed herein can be formulated into a variety of forms and administered by a number of different means. A pharmaceutical formulation can be administered orally, rectally, or parenterally, in formulations containing conventionally acceptable carriers, adjuvants, and vehicles as desired. The term “parenteral” as used herein can include subcutaneous, intravenous, intramuscular, or intrasternal injection and infusion techniques. Administration can include injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), transdermal, transmucosal, sublingual, buccal and topical (including epicutaneous, dermal, enema, eye drops, ear drops, intranasal, vaginal) administration. In some exemplary embodiments, a route of administration can be via an injection such as an intramuscular, intravenous, subcutaneous, or intraperitoneal injection.

In some embodiments, the method of administration can be local administration of the peptide or formulation. In some embodiments, local administration comprises washing, irrigating, debridement, or a combination thereof of a site of infection. In some embodiments, a site of infection is an open wound, an implant (in vivo or ex vivo), a joint, or topical. In some embodiments, a site of infection is an implant or a joint.

In some embodiments, washing or irrigation of a site of infection may occur over a time period of from at about 0.5 minute (min) to at about 600 min. In some embodiments, washing or irrigation of a site of infection may occur over a time period of at least about 1 min, 2 min, 5 min, 10 min, 15 min, 20 min, 30 min, 45 min, 1 hour, 2 hours, 3 hours, and any increments there between. In some embodiment, washing or irrigation of a site of infection may occur over 15 min. In some embodiment, washing or irrigation of a site of infection may occur over 30 min.

In some embodiments, the administration of a formulation described herein can be after a surgical procedure or before, during, or after a care regiment of a surgical procedure (e.g., debridement, antibiotics, and imponent retention (DAIR)).

Solid dosage forms for oral administration can include capsules, tablets, caplets, pills, troches, lozenges, powders, and granules. A capsule can comprise a core material comprising a nutritive protein or formulation and a shell wall that encapsulates a core material. In some embodiments a core material can comprise at least one of a solid, a liquid, and an emulsion. In some embodiments a shell wall material can comprise at least one of a soft gelatin, a hard gelatin, and a polymer. Suitable polymers can include but not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer can function as taste-masking agents. Tablets, pills, and the like can be compressed, multiply compressed, multiply layered, and/or coated. A coating can be single or multiple. In some embodiments, a coating material can comprise at least one of a saccharide, a polysaccharide, and glycoproteins extracted from at least one of a plant, a fungus, and a microbe. Non-limiting examples can include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments a coating material can comprise a protein. In some embodiments, a coating material can comprise at least one of a fat and/or an oil. In some embodiments the at least one of a fat and/or an oil can be high temperature melting. In some embodiments the at least one of a fat and/or an oil can be hydrogenated or partially hydrogenated. In some embodiments the at least one of a fat and/or an oil can be derived from a plant. In some embodiments the at least one of a fat and/or an oil can comprise at least one of glycerides, free fatty acids, and fatty acid esters. In some embodiments a coating material can comprise at least one edible wax. An edible wax can be derived from animals, insects, or plants. Non-limiting examples can include beeswax, lanolin, bayberry wax, carnauba wax, and rice bran wax. Tablets and pills can additionally be prepared with enteric coatings. In some embodiments, a tablet, pill, and the like can be formulated for an extended release profile.

In some embodiments, a peptide described herein or pharmaceutically acceptable salt thereof can be administered in a formulation for topical administration. For topical administration, an active agent may be formulated as is known in the art for direct application to a target area. Forms chiefly conditioned for topical application can take the form, for example, of creams, milks, gels, powders, dispersion or microemulsions, lotions thickened to a greater or lesser extent, impregnated pads, ointments or sticks, soaps, detergents, lotions or cakes of soap. Other conventional forms for this purpose include wound dressings, coated bandages or other polymer coverings, ointments, creams, lotions, pastes, jellies. Thus, a therapeutic peptide disclosed herein can be delivered via patches or bandages for dermal administration. Alternatively, a peptide can be formulated to be part of an adhesive polymer, such as polyacrylate or acrylate/vinyl acetate copolymer. For long-term applications it might be desirable to use microporous and/or breathable backing laminates, so hydration or maceration of a skin can be minimized. A backing layer can be any appropriate thickness that will provide a desired protective and support functions. A suitable thickness will generally be from about 1 to about 1000 microns. For example, from about 10 to about 300 microns. Topical administration may be in the form of a nail coating or lacquer. For example, an antifungal peptide can be formulated in a solution for topical administration that contains ethyl acetate (NF), isopropyl alcohol (USP), and butyl monoester of poly[methylvinyl ether/maleic acid] in isopropyl alcohol. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Liquid formulations can include a syrup (for example, an oral formulation), an intravenous formulation, an intranasal formulation, an ocular formulation (e.g., for treating an eye infection), an otic formulation (e.g., for treating an ear infection), an ointment, a cream, an aerosol, and the like. In some embodiments, a combination of various formulations can be administered.

Drops, such as eye drops or nose drops, may be formulated with one or more peptide(s) in an aqueous or non-aqueous base also comprising one or more dispersing agents, solubilizing agents or suspending agents. Drops can be delivered via a simple eye dropper-capped bottle, via a plastic bottle adapted to deliver liquid contents drop-wise, or via a specially shaped closure.

In some embodiments, the method of administration may last over a course of at least about 1 hour, 5 hours, 12 hours, 24 hours, 48 hours, 72 hours, 4 days, 5 days, 1 week, 2 weeks, 3 week, 4 weeks, 2 months, 3 months, 4 months, 5 months, 6 months, 7 months, 8 months, 9 months, 10 months, 11 months, 12 months, 2 years, 3 years, 4 years, 5 years, 6 years, 7 years, 8 years, 9 years, 10 years, 20 years, 25 years, 30 years, 35 years, 40 years, 45 years, 50 years, 55 years, 60 years, 65 years, 70 years, 75 years, or 80 years.

Administration of a peptide, pharmaceutically acceptable salt thereof, or a formulation comprising a peptide or salt thereof to a subject can be used to at least partially ameliorate a bacterial infection in a subject. Administration of a peptide, pharmaceutically acceptable, or formulation can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some embodiments, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration of a peptide, pharmaceutically acceptable, or formulation can be performed at least 1, 2, 3, 4, 5, 6, 7, 8, 9, 10, 11, 12, 13, 14, 15, 16, 17, 18, 19, 20, 21, 22, 23, or 24 times a day. In some embodiments, administration of a peptide, pharmaceutically acceptable, or formulation can be performed at least 1, 2, 3, 4, 5, 6, 7, 8, 9, 10, 11, 12, 13, 14, 15, 16, 17, 18, 19, 20, or 21 times a week. In some embodiments, administration of a peptide, pharmaceutically acceptable, or formulation can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some embodiments, administration of the pharmaceutical formulation comprising a peptide or pharmaceutically acceptable salt occurs over a time period of from at least about 0.5 min to at least about 1 min, from at least about 1 min to at least about 2 min, from at least about 2 min to at least about 3 min, from at least about 3 min to at least about 4 min, from at least about 4 min to at least about 5 min, from at least about 5 min to at least about 6 min, from at least about 6 min to at least about 7 min, from at least about 7 min to at least about 8 min, from at least about 8 min to at least about 9 min, from at least about 9 min to at least about 10 min, from at least about 10 min to at least about 11 min, from at least about 11 min to at least about 12 min, from at least about 12 min to at least about 13 min, from at least about 13 min to at least about 14 min, from at least about 14 min to at least about 15 min, from at least about 15 min to at least about 16 min, from at least about 16 min to at least about 17 min, from at least about 17 min to at least about 18 min, from at least about 18 min to at least about 19 min, from at least about 19 min to at least about 20 min, from at least about 21 min to at least about 22 min, from at least about 22 min to at least about 23 min, from at least about 23 min to at least about 24 min, from at least about 24 min to at least about 25 min, from at least about 25 min to at least about 26 min, from at least about 26 min to at least about 27 min, from at least about 27 min to at least about 28 min, from at least about 28 min to at least about 29 min, or from at least about 29 min to at least about 30 min.

A peptide disclosed herein, salt thereof, or a formulation containing a peptide or salt thereof can be administered to a subject in order to at least partially ameliorate a disease or condition. A subject can be in need of a treatment of a disease or condition. In some embodiments, a subject may have been previously diagnosed with a disease or condition described herein, and/or may be at risk of developing a disease or condition as described herein.

### Coatings

Also disclosed herein are methods of producing a coating comprising a peptide disclosed herein, salt thereof, or a formulation containing a peptide or salt thereof. A coating can be an antimicrobial coating that can be applied to a surface to remove contaminants from a surface, or to prevent contamination in the first instance. A coating can comprise an antimicrobial peptide disclosed herein. A coating can generally be prepared by contacting a coating material with a peptide disclosed herein, salt thereof, or a formulation containing a peptide or salt thereof.

In some embodiments, a coating can be in the form of a film, sheet, or liquid used to coat a biological or non-biological surface. A film can be prepared by coating material capable of producing a film with a peptide disclosed herein, salt thereof, or a formulation containing a peptide or salt thereof. A coating material capable of producing a film can be an adhesive compound, such as a mucoadhesive, used to bind a compound to a biological surface. An exemplary mucoadhesive can be a highly negatively charged polymer such as polycarbophil. A coating material capable of producing a film can be adhered to a biological surface to treat or prevent an infection on a biological surface. For example, a peptide described herein can be formulated as a coating for adherence onto an open wound, thereby eliminating a need for a bandage by directly adhering an antimicrobial compound to a site of action. Further applications can include adhering a coating onto a transplanted organ to prevent infection by a pathogen during a transplant process.

In some embodiments, a coating can comprise a peptide disclosed herein, salt thereof, or a formulation containing a peptide or salt thereof can be used to sterilize a surface. For example, a coating can be applied to surgical equipment, and any surface in contact with surgical equipment, prior to an operation. Such practice can mitigate a risk contamination of the surgical equipment during transport. Scientific equipment can also be coated with such a coating to prevent cross contamination of certain microbes that could interfere with a measurement to be taken with the equipment.

Further examples of the use of a coating containing a peptide described herein can include coating an article such as a medical device. In some embodiments, the medical device can be an implantable medical device. For example, a medical device such as a catheter or prosthetic limb can be coated with a coating as described above to prevent contamination during packaging, storage, or during a transplant operation. In some embodiments, a peptide can be the sole antimicrobial compound in a coating. In other instances, a coating can comprise other antimicrobial compounds such as those described herein. Metallic antimicrobial compounds such as silver nitrate can also be used in combination with a peptide scribed herein.

An article for implant in contact with a coating containing a peptide, pharmaceutically acceptable salt thereof, or pharmaceutical formulation can be assembled as a formulation containing an article and coating.

### Dosage

In some embodiments, the pharmaceutical formulations described herein is in the form of a unit dose. In some embodiments, a pharmaceutical formulation can be formulated to optimize pharmacokinetics/pharmacodynamics of a peptide or salt thereof contained therein.

In some embodiments, a peptide, pharmaceutically acceptable salt thereof, or pharmaceutical formulation comprising a peptide or salt thereof described herein can be administered at a dose of from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg. In some embodiments, a peptide, pharmaceutically acceptable salt thereof, or pharmaceutical formulation comprising a peptide or salt thereof described herein can be administered ata dose of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg.

In some embodiments, pharmaceutical formulation comprising a peptide or pharmaceutically acceptable salt is present at a concentration from at least about 0.01 μg/mL to at least about 100 mg/mL. In some embodiments, the peptide or pharmaceutically acceptable salt is present at a concentration from at least about at least about 0.1 mg/mL to at least about 5 mg/mL. In some embodiments, the peptide or pharmaceutically acceptable salt is present at a concentration from at least about at least about 0.5 mg/mL to at least about 1 mg/mL. In some embodiments, the peptide or pharmaceutically acceptable salt is present at a concentration about 1 mg/mL. In some embodiments, the peptide or pharmaceutically acceptable salt is present at a concentration about 2 mg/mL. In some embodiments, the peptide or pharmaceutically acceptable salt is present at a concentration about 3 mg/mL. In some embodiments, the peptide or pharmaceutically acceptable salt is present at a concentration about 4 mg/mL. In some embodiments, the peptide or pharmaceutically acceptable salt is present at a concentration about 5 mg/mL. In some embodiments, the peptide or pharmaceutically acceptable salt is present at a concentration about 6 mg/mL. In some embodiments, the peptide or pharmaceutically acceptable salt is present at a concentration about 7 mg/mL. In some embodiments, the peptide or pharmaceutically acceptable salt is present at a concentration about 8 mg/mL. In some embodiments, the peptide or pharmaceutically acceptable salt is present at a concentration about 9 mg/mL. In some embodiments, the peptide or pharmaceutically acceptable salt is present at a concentration about 10 mg/mL. In some embodiments, the peptide or pharmaceutically acceptable salt is present at a concentration about 20 mg/mL. In some embodiments, the peptide or pharmaceutically acceptable salt is present at a concentration about 30 mg/mL. In some embodiments, the peptide or pharmaceutically acceptable salt is present at a concentration about 40 mg/mL. In some embodiments, the peptide or pharmaceutically acceptable salt is present at a concentration about 40.7 mg/mL. In some embodiments, the peptide or pharmaceutically acceptable salt is present at a concentration about 50 mg/mL. In some embodiments, the peptide or pharmaceutically acceptable salt is present at a concentration about 60 mg/mL. In some embodiments, the peptide or pharmaceutically acceptable salt is present at a concentration about 70 mg/mL. In some embodiments, the peptide or pharmaceutically acceptable salt is present at a concentration about 80 mg/mL. In some embodiments, the peptide or pharmaceutically acceptable salt is present at a concentration about 81.4 mg/mL. In some embodiments, the peptide or pharmaceutically acceptable salt is present at a concentration about 90 mg/mL. In some embodiments, the peptide or pharmaceutically acceptable salt is present at a concentration about 100 mg/mL.

In some embodiments, pharmaceutical formulation comprising a peptide or pharmaceutically acceptable salt can exhibit antimicrobial activity against an infection at a concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some embodiments, effective amounts of a peptide or pharmaceutically acceptable salt for treating or preventing an infection can be a concentration from at least about 0.01 μg/mL to at least about 100 mg/mL. In some embodiments, effective amounts of a peptide or pharmaceutically acceptable salt for treating or preventing an infection is at a concentration from at least about at least about 0.1 mg/mL to at least about 5 mg/mL. In some embodiments, effective amounts of a peptide or pharmaceutically acceptable salt for treating or preventing an infection is at a concentration from at least about at least about 0.5 mg/mL to at least about 1 mg/mL. In some embodiments, effective amounts of a peptide or pharmaceutically acceptable salt for treating or preventing an infection is at a concentration from at least about at least about 1 mg/mL to at least about 10 mg/mL. In some embodiments, effective amounts of a peptide or pharmaceutically acceptable salt for treating or preventing an infection is at a concentration from at least about at least about 3 mg/mL to at least about 10 mg/mL. In some embodiments, effective amounts of a peptide or pharmaceutically acceptable salt for treating or preventing an infection is at a concentration from at least about at least about 1 mg/mL to at least about 100 mg/mL.

In some embodiments, effective amounts of a peptide or pharmaceutically acceptable salt for treating or preventing an infection is at a concentration about 1 mg/mL. In some embodiments, the effective amounts of a peptide or pharmaceutically acceptable salt for treating or preventing an infection is at a concentration about 2 mg/mL. In some embodiments, the effective amounts of a peptide or pharmaceutically acceptable salt for treating or preventing an infection is at a concentration about 3 mg/mL. In some embodiments, the effective amounts of a peptide or pharmaceutically acceptable salt for treating or preventing an infection is at a concentration about 4 mg/mL. In some embodiments, the effective amounts of a peptide or pharmaceutically acceptable salt for treating or preventing an infection is at a concentration about 5 mg/mL. In some embodiments, the effective amounts of a peptide or pharmaceutically acceptable salt for treating or preventing an infection is at a concentration about 6 mg/mL. In some embodiments, the effective amounts of a peptide or pharmaceutically acceptable salt for treating or preventing an infection is at a concentration about 7 mg/mL. In some embodiments, the effective amounts of a peptide or pharmaceutically acceptable salt for treating or preventing an infection is at a concentration about 8 mg/mL. In some embodiments, the effective amounts of a peptide or pharmaceutically acceptable salt for treating or preventing an infection is at a concentration about 9 mg/mL. In some embodiments, the effective amounts of a peptide or pharmaceutically acceptable salt for treating or preventing an infection is at a concentration about 10 mg/mL. In some embodiments, the effective amounts of a peptide or pharmaceutically acceptable salt for treating or preventing an infection is at a concentration about 20 mg/mL. In some embodiments, the effective amounts of a peptide or pharmaceutically acceptable salt for treating or preventing an infection is at a concentration about 50 mg/mL. In some embodiments, the effective amounts of a peptide or pharmaceutically acceptable salt for treating or preventing an infection is at a concentration about 100 mg/mL.

In some embodiments, effective amounts of a peptide or pharmaceutically acceptable salt for treating or preventing an infection may be a concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some embodiments, effective amounts of a peptide or pharmaceutically acceptable salt for treating or preventing an infection may be from at least about 1 μL to at least about 2 μL, from at least about 2 μL to at least about 3 μL, from at least about 3 μL to at least about 4 μL, from at least about 4 μL to at least about 5 μL, from at least about 5 μL to at least about 6 μL, from at least about 6 μL to at least about 7 μL, from at least about 7 μL to at least about 8 μL, from at least about 8 μL to at least about 9 μL, from at least about 9 μL to at least about 10 μL, from at least about 10 μL to at least about 20 μL, from at least about 20 μL to at least about 30 μL, from at least about 30 μL to at least about 40 μL, from at least about 40 μL to at least about 50 μL, from at least about 50 μL to at least about 60 μL, from at least about 60 μL to at least about 70 μL, from at least about 70 μL to at least about 80 μL, from at least about 80 μL to at least about 90 μL, from at least about 90 μL to at least about 100 μL, from at least about 100 μL to at least about 200 μL, from at least about 200 μL to at least about 300 μL, from at least about 300 μL to at least about 400 μL, from at least about 400 μL to at least about 500 μL, from at least about 500 μL to at least about 600 μL, from at least about 600 μL to at least about 700 μL, from at least about 700 μL to at least about 800 μL, from at least about 800 μL to at least about 900 μL, from at least about 900 μL to at least about 1 mL, from at least about 1 mL to at least about 2 mL, from at least about 2 mL to at least about 3 mL, from at least about 3 mL to at least about 4 mL, from at least about 4 mL to at least about 5 mL, from at least about 5 mL to at least about 6 mL, from at least about 6 mL to at least about 7 mL, from at least about 7 mL to at least about 8 mL, from at least about 8 mL to at least about 9 mL, from at least about 9 mL to at least about 10 mL, from at least about 10 mL to at least about 20 mL, from at least about 20 mL to at least about 30 mL, from at least about 30 mL to at least about 40 mL, from at least about 40 mL to at least about 50 mL, from at least about 50 mL to at least about 60 mL, from at least about 60 mL to at least about 70 mL, from at least about 70 mL to at least about 80 mL, from at least about 80 mL to at least about 90 mL, from at least about 90 mL to at least about 100 mL, from at least about 100 mL to at least about 200 mL, from at least about 200 mL to at least about 300 mL, from at least about 300 mL to at least about 400 mL, from at least about 400 mL to at least about 500 mL, from at least about 500 mL to at least about 600 mL, from at least about 600 mL to at least about 700 mL, from at least about 700 mL to at least about 800 mL, from at least about 800 mL to at least about 900 mL, from at least about 900 mL to at least about 1 L, from at least about 1 L to at least about 2 L, from at least about 2 L to at least about 3 L, from at least about 3 L to at least about 4 L, from at least about 4 L to at least about 5 L, from at least about 5 L to at least about 6 L, from at least about 6 L to at least about 7 L, from at least about 7 L to at least about 8 L, from at least about 8 L to at least about 9 L, from at least about 9 L to at least about 10 L, from at least about 10 L to at least about 20 L, from at least about 20 L to at least about 30 L, from at least about 30 L to at least about 40 L, from at least about 40 L to at least about 50 L, from at least about 50 L to at least about 60 L, from at least about 60 L to at least about 70 L, from at least about 70 L to at least about 80 L, from at least about 80 L to at least about 90 L, from at least about 90 L to at least about 100 L, from at least about 100 L to at least about 200 L, from at least about 200 L to at least about 300 L, from at least about 300 L to at least about 400 L, from at least about 400 L to at least about 500 L, from at least about 500 L to at least about 600 L, from at least about 600 L to at least about 700 L, from at least about 700 L to at least about 800 L, from at least about 800 L to at least about 900 L, from at least about 900 L to at least about 1 kL, from at least about 1 kL to at least about 2 kL, from at least about 2 kL to at least about 3 kL, from at least about 3 kL to at least about 4 kL, from at least about 4 kL to at least about 5 kL, from at least about 5 kL to at least about 6 kL, from at least about 6 kL to at least about 7 kL, from at least about 7 kL to at least about 8 kL, from at least about 8 kL to at least about 9 kL, or from at least about 9 kL to at least about 10 kL.

In some embodiments, the pharmaceutical formulation is physiologically isotonic, physiologically hypotonic, or physiologically hypertonic. In some embodiments, the pharmaceutical formulation is physiologically isotonic. In some embodiments, the pharmaceutical formulation is physiologically hypotonic. In some embodiments, the pharmaceutical formulation is physiologically hypertonic. In some embodiments, the pharmaceutical formulation may have an osmolarity of from at least 1 milliosmoles per kilogram (mOsm/kg) to at least 10 osmoles per kilogram (Osm/kg), at least 1 mOsm/kg to at least 5 Osm/kg, at least 1 mOsm/kg to at least 4 Osm/kg, at least 1 mOsm/kg to at least 3 Osm/kg, at least 1 mOsm/kg to at least 2 Osm/kg, at least 1 mOsm/kg to at least 1 Osm/kg, at least 1 mOsm/kg to at least 900 mOsm/kg, at least 1 mOsm/kg to at least 800 mOsm/kg, at least 1 mOsm/kg to at least 700 mOsm/kg, at least 1 mOsm/kg to at least 600 mOsm/kg, at least 1 mOsm/kg to at least 500mOsm/kg, at least 1 mOsm/kg to at least 400 mOsm/kg, at least 1 mOsm/kg to at least 300mOsm/kg, at least 1 mOsm/kg to at least 200 mOsm/kg, at least 1 mOsm/kg to at least 100mOsm/kg, at least 1 mOsm/kg to at least 500 mOsm/kg, at least 1 mOsm/kg to at least 30 mOsm/kg, at least 30 mOsm/kg to at least 400 mOsm/kg, at least 275 mOsm/kg to at least 295 mOsm/kg, at least 30 mOsm/kg to at least 800 mOsm/kg, at least 50 mOsm/kg to at least 500 mOsm/kg, at least 100 mOsm/kg to at least 500 mOsm/kg, or at least 100 mOsm/kg to at least 200 mOsm/kg. In some embodiments, the pharmaceutical formulation may have an osmolarity of from at least 30 mOsm/kg to at least 400 mOsm/kg. In some embodiments, the pharmaceutical formulation may have an osmolarity of from at least 275 mOsm/kg to at least 295 mOsm/kg. In some embodiments, the pharmaceutical formulation has a total osmolarity ranging from about 1 mOsm/L to about 5,000 mOsm/L. In some embodiments, the pharmaceutical formulation has a total osmolarity of about 1 mOsm/L, about 50 mOsm/L, about 100 mOsm/L, about 150 mOsm/L, about 200 mOsm/L, about 250 mOsm/L, about 300 mOsm/L, about 350 mOsm/L, about 400 mOsm/L, about 450 mOsm/L, about 500 mOsm/L, about 1000 mOsm/L, about 1500 mOsm/L, about 2000 mOsm/L, about 2500 mOsm/L, about 3000 mOsm/L, about 3500 mOsm/L, about 4000 mOsm/L, about 4500 mOsm/L, or about 5000 mOsm/L. In some embodiment, the pharmaceutical formulation has a total osmolality ranging from about 1 milliosmole per kilogram (mOsm/kg) from 5000 mOsm/kg. In some embodiments, the pharmaceutical formulation has a total osmolarity of about 1 mOsm/kg, about 50 mOsm/kg, about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, about 500 mOsm/kg, about 1000 mOsm/kg, about 1500 mOsm/kg, about 2000 mOsm/kg, about 2500 mOsm/kg, about 3000 mOsm/kg, about 3500 mOsm/kg, about 4000 mOsm/kg, about 4500 mOsm/kg, or about 5000 mOsm/kg. In some embodiments, the pharmaceutical formulation has a total ionic strength ranging from about 0.001 molar (M) and 1.0 M. The aqueous carrier may have a total ionic strength of about 0.001 M, about 0.01 M, about 0.015 M, about 0.02 M, about 0.025 M, about 0.03 M, about 0.035 M, about 0.04 M, about 0.05 M, about 0.055 M, about 0.06 M, about 0.065 M, about 0.07 M, about 0.075 M, about 0.08 M, about 0.085 M, about 0.09 M, about 0.1 M, about 0.12 M, about 0.14 M, about 0.15 M, about 0.16 M, about 0.18 M, about 0.2 M, about 0.22 M, about 0.24 M, about 0.25 M, about 0.26 M, about 0.28 M, about 0.03 M, about 0.35 M, about 0.4 M, about 0.45 M, about 0.5 M, about 0.55 M, about 0.6 M, about 0.65 M, about 0.7 M, about 0.75 M about 0.8 M, about 0.85 M, about 0.9 M, about 0.95 M, or about 1.0 M.

### Combination Administration

Also contemplated are combination products that include one or more peptides disclosed herein and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein.

In addition, it is contemplated that a peptide can be combined with topical antifungal agents such as ciclopirox olamine, haloprogin, tolnaftate, undecylenate, topical nysatin, amorolfine, butenafine, naftifine, terbinafine, and other topical agents. In some embodiments, a pharmaceutical formulation can comprise an additional agent. In some embodiments, an additional agent can be present in a therapeutically effective amount in a pharmaceutical formulation. In some embodiments, an additional pharmaceutical agent can be an antibiotic agent. An antibiotic agent can of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides and tetracycline. Alternatively, or additionally an antibiotic agent may be effective against mycobacteria. In some embodiments, an antibiotic agent may be an aminoglycoside such as Amikacin, Gentamicin, Kanamycin, Neomycin, Netilmicin, Tobramycin or Paromomycin. According to one embodiment, an antibiotic agent may be an Ansamycin such as Geldanamycin and Herbimycin. In some embodiments, an antibiotic agent may be a carbacephem such as Loracarbef. In some embodiments, an antibiotic agent can be a carbapenem such as Ertapenem, Doripenem, Imipenem/Cilastatin or Meropenem.

In some embodiments, an antibiotic agent may be a beta lactam antibiotic or pharmaceutically acceptable salt thereof may include but are not limited to Cephalexin, Cephradine, Cefadroxil, Cefazolin, B-lactam antibiotic C, Cephalothin, Cefapirin, Cefuroxime, Cefprozil, Loracarbef, Cefuroxime, Cefoxitin, Cefotetan, Cefaclor, Cefamandole, Ceftriaxone, Cefdinir, Cefixime, Cefpodoxime, Cefditoren, Ceftibuten, Ceftazidime, Cefotaxime, Cefoperazone, Ceftizoxime, Cefepime, Cefiderocol, Cefpirome, Ceftaroline, Benzathine, Benzylpenicillin, Phenoxymethylpenicillin, Procaine penicillin, Pheneticillin, Cloxacillin, Dicloxacillin, Flucloxacillin, Methicillin, Nafcillin, Oxacillin, Temocillin, Amoxillin, Ampicillin, Mecillinam, Piperacillin, Carbenicillin, Ticarillin, Carbenicillin, Ticarcillin, Azlocillin, Mezlocillin, Piperacillin, Biapenem, Doripenem, Ertapenem, Faropenem, Imipenem, Meropenem, Panipenem, Razupenem, Tebipenem, Thienamycin, Aztreonam, Tigermonam, Nocardicin A, Tabtoxinine beta-lactam, Clavulanic acid, Tazobactam, Sulbactam, or Avibactam. In some embodiments, an antibiotic agent may be a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. Alternatively, an antibiotic agent may be a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime and Ceftobiprole. In some embodiments, an antibiotic agent may be a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin. In some embodiments, an antibiotic agent may be a monobactams such as Aztreonam, or a nitrofuran such as Furazolidone or Nitrofurantoin. In some embodiments, an antibiotic agent may be a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin. In some embodiments, an antibiotic agent may be a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, and Trimethoprim-Sulfamethoxazole (Co-trimoxazole) (TMP-SMX). In some embodiments, an antibiotic agent may be a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin. In some embodiments, an antibiotic agent may be a polypeptide such as Bacitracin, Colistin and Polymyxin B. In some embodiments, an antibiotic agent may be a tetracycline such as Demeclocycline, Doxycycline, Minocycline and Oxytetracycline. In some embodiments, an antibiotic agent may be effective against mycobacteria. An antibiotic agent may be Clofazimine, Lamprene, Dapsone, Capreomycin, Cycloserine, Ethambutol, Ethionamide, Isoniazid, Pyrazinamide, Rifampicin, Rifabutin, Rifapentine or Streptomycin. In some exemplary embodiments, an antibiotic agent can include Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Methicillin, Linezolid, a Streptogramin, Tigecycline, Daptomycin, a salt of any of these, or any combination thereof.

In some embodiments, an additional pharmaceutical agent can be an antimicrobial agent disclosed herein. In some embodiments, an antimicrobial agent can be cysteamine or a salt thereof. While cysteamine can be typically used to treat conditions such as cystinosis that are not derived from an infection, the use of cysteamine as an antimicrobial compound has shown promise. For example, WO2010112848 describes the use of formulations containing cysteamine for as antimicrobial agents capable of inhibiting the formation of a bacterial biofilm for a broad range of bacterial strains, including Pseudomonas spp., Staphylococcus spp., Haemophilus spp., Burkholderia spp., Streptococcus spp., Propionibacterium spp.

In some embodiments, an additional pharmaceutical agent can be an antiviral agent. In some embodiments, an antiviral agent can be Acyclovir, Brivudine, Cidofovir, Docosanol, Famciclovir, Foscarnet, Fomivirsen, Ganciclovir, Idoxuridine, Penciclovir, Peramivir, Trifluridine, Valacyclovir, Vidarabine, Lamivudine, Ribavirin Amantadine, Rimantadine, a neuraminidase inhibitor, Oseltamivir, Zanamivir, a salt of any of these, or any combination thereof.

In some embodiments, an additional pharmaceutical agent can be an antineoplastic. In some embodiments, an antineoplastic can be selected from the group consisting of cyclophosphamide, methotrexate, 5-fluorouracil, doxorubicin, procarbazine, prednisolone, bleomycin, vinblastine, dacarbazine, cisplatin, epirubicin, a salt of any of these, and any combination thereof.

In some embodiments, a peptide disclosed herein, salt thereof, or a formulation containing a peptide or salt thereof can be an antiviral agent.

In some embodiments, a peptide, pharmaceutically acceptable, or formulation can be administered in combination with an antibiotic or an additional antiviral agent disclosed herein.

### Infection

Provided herein are pharmaceutical formulations comprising a peptide and method of treating or preventing a disease or condition comprising administering the pharmaceutical formulation. In some embodiments, the condition or disease is an infection. In other embodiments, the infection is a microbial infection. In some embodiments, the infection is a bacterial infection, viral infection, fungal infection, or a combination thereof.

In some embodiments, bacterial infection may be derived from a bacterial species selected from the group, but not exclusive to the group, consisting of: Staphylococcus spp., e.g. Staphylococcus aureus (e.g. Staphylococcus aureus NCTC 10442 and Staphylococcus aureus ATCC25923), Staphylococcus epidermidis; Chlamydia spp., e.g. Chlamydia trachomatis, Chlamydia pneumoniae, Chlamydia psittaci; Enterococcus spp., e.g. Enterococcus faecalis; Streptococcus pyogenes; Listeria spp.; Pseudomonas spp.; Mycobacterium spp., e.g. Mycobacterium tuberculosis complex; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori, Helicobacter fells,; Neisseria spp., e.g. Neisseria gonorrhoea, Neisseria meningitidis; Borrelia burgdorferi; Shigella spp., e.g. Shigella jlexneri; Escherichia coli (E.coli 0157:H7 NCTC 12900); Haemophilus spp., e.g. Haemophilus influenzae; Francisella tularensis; Bacillus spp., e.g. Bacillus anthraces; Clostridia spp., e.g. Clostridium botulinum, Clostridium difficile; Yersinia spp., e.g. Yersinia pestis; Treponema spp.; Burkholderia spp., e.g. Burkholderia cepacia complex, B. mallei, B pseudomallei; Propionibacterium spp., e.g. P. acnes, Acinetobacter species, an Actinomyces species, a Campylobacter species, a Candida species, Corynebacterium minutissium, Corynebacterium pseudodiphtheriae, Corynebacterium stratium, Corynebacterium group G1, Corynebacterium group G2, Enterobacteriaceae, anEnterococcus species, Klebsiella pneumoniae, a Moraxella species, a non-tuberculous mycobacteria species, a Porphyromonas species, Prevotella melaninogenicus, Salmonella typhimurium, Serratia marcescens Streptococcus agalactiae, Staphylococcus salivarius, Streptococcus mitis, Streptococcus sanguis, Streptococcus pneumoniae, Vibrio cholerae, a Coccidioides species, or a Cryptococcus species. In some embodiments, a peptide or pharmaceutically acceptable salt thereof described herein can reduce infection of bacteria against at least one of Staphylococcus aureus, methicillin resistant Staphylococcus aureus, Streptococcus pneumonia, carbapenem-resistant Enteroacteriaceae, Staphylococcus epidermidis, Staphylococcus salivarius, Corynebacterium minutissium, Corynebacterium pseudodiphtheriae, Corynebacterium stratium, Corynebacterium group G1, Corynebacterium group G2, Streptococcus pneumonia, Streptococcus mitis, Streptococcus sanguis, Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaninogenicus, Helicobacter pylori, Helicobacter felis, or Campylobacter jejuni. In some embodiments, bacterial infection may be derived from a bacterial species selected from the group Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, and any combination thereof. In some embodiments, the bacterial may be antibiotic-tolerant or antibiotics-resistant. A bacterial strain can also be an antibiotic-resistant variant or a bacterial strain described herein. In some embodiments, a bacterial strain can be resistant to an antibiotic described herein. In some embodiments, a bacterial strain can be resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or any combination thereof.

A microbial biofilm also referred to as a biological biofilm, can be a community of microbial cells embedded in an extracellular matrix of polymeric substances and adherent to a biological or a non-biotic surface. A range of microorganisms (bacteria, fungi, and/or protozoa, with associated bacteriophages and other viruses) can be found in these biofilms. Biofilms are ubiquitous in nature, are commonly found in a wide range of environments. Biofilms are being increasingly recognized by the scientific and medical community as being implicated in many infections, and especially their contribution to the recalcitrance of infection treatment. Biofilms can be etiologic agents for a number of disease states in mammals and are involved in 80% of infections in humans. Examples can include skin and wound infections, middle-ear infections, gastrointestinal tract infections, peritoneal membrane infections, urogenital tract infections, oral soft tissue infections, formation of dental plaque, eye infections (including contact lens contamination), endocarditis, infections in cystic fibrosis, and infections of indwelling medical devices such as joint prostheses, dental implants, catheters and cardiac implants. Microbes in biofilms can be significantly more resistant to antimicrobial treatment than their planktonic counterparts. Biofilm formation is not limited solely to the ability of microbes to attach to a surface. Microbes growing in a biofilm can interact more between each other than with the actual physical substratum on which the biofilm initially developed. The suggested mechanisms by which biofilm-associated microorganisms elicit diseases in their host can include the following: (i) delayed penetration of the antimicrobial agent through the biofilm matrix, (ii) detachment of cells or cell aggregates from indwelling medical device biofilms, (iii) production of endotoxins, (iv) resistance to the host immune system, (v) provision of a niche for the generation of resistant organisms through horizontal gene transfer of antimicrobial resistance &/or virulence determinant genes, and (vi) altered growth rate (i.e. metabolic dormancy).

In some embodiments, the infection is periprosthetic joint infection. In some embodiments, periprosthetic joint infection is caused by a bacterial infection. In some embodiments, the periprosthetic joint infection further comprises a biofilm. In some embodiments, the infection arises after a surgical procedure (e.g., implant of a prosthesis or surgical site infection). Exemplary surgical procedures can include, but not limited to, total knee arthroplasty, joint replacement surgery, and implant of a prosthesis.

In some embodiments, bacteria, fungi, and/or protozoa, with associated bacteriophages and other viruses described herein can secrete a biofilm. In some embodiments, bacteria, fungi, and/or protozoa, with associated bacteriophages and other viruses described herein can form a biofilm. A peptide, pharmaceutically acceptable salt thereof described herein, or a formulation comprising a peptide or salt thereof described herein can be administered to at least partially penetrate, inhibit formation of, or destroy a biological biofilm. In some embodiments, additional agents can be added to at least partially inhibit formation of, or destroy, a biological biofilm.

In some embodiments, the infection is a viral infection. In some embodiments, a virus can be a DNA virus, a RNA virus, or a reverse transcriptase (retro) virus. A virus can be a dsDNA (double stranded DNA) virus, a ssDNA (single stranded DNA) virus, a dsRNA (double stranded RNA) virus, a +ssRNA (+strand or sense single stranded RNA) virus, a −ssRNA (−strand or antisense RNA) virus, a ssRNA-RT (single stranded RNA reverse transcriptase) virus, or a dsDNA-RT (double stranded DNA reverse transcriptase) virus. As described herein, a peptide described herein can be engineered to disrupt the integrity of a viral envelope of an enveloped virus. Such a disruption can at least partially reduce a viability of a virus, which can ameliorate an infection brought about by a virus. A virus may be derived from the group, but not exclusive to the group, of a herpesvirus, a poxvirus, a hepadnavirus, a flavivirus, a togavirus, a coronavirus, hepatitis C, hepatitis D, an orthomyxovirus, a papillomavirus, a polyomaviridae, a parvovirus, a cytomegalovirus, an Epstein-Barr virus, a small pox virus, a cow pox virus, a sheep pox virus, an orf virus, a monkey pox virus, a vaccinia virus, a paramyxovirus, a retrovirus, an adenovirus, a rhabdovirus, a bunyavirus, a filovirus, an alphavirus, an arenavirus, a lentivirus, and any combination thereof. In some embodiments, the virus can be an enveloped virus. Examples of an enveloped viruses can include: a poxvirus, a hepadnavirus, a flavivirus, a togavirus, a coronavirus, hepatitis C, hepatitis D, an orthomyxovirus, a cytomegalovirus, an Epstein-Barr virus, a small pox virus, a cow pox virus, a sheep pox virus, an orf virus, a monkey pox virus, a vaccinia virus, a rhabdovirus, a bunyavirus, a filovirus, an alphavirus, an arenavirus, a lentivirus, and the like.

Also envisaged are treatments of fungal, protozoal, or other parasitic infections by administration of a peptide described herein, salt thereof, or formulation containing a peptide or salt thereof. In some embodiments, a pathogen can be a drug-resistant fungal, protozoal, or other parasitic organism.

A parasitic pathogen may be derived from a parasite selected from, but not limited to, the group consisting of Trypanosoma spp. (Trypanosoma cruzi, Trypansosoma brucei), Leishmania spp., Giardia spp., Trichomonas spp., Entamoeba spp., Naegleria spp., Acanthanioeba spp., Schistosoma spp., Plasmodium spp., Crytosporidium spp., Isospora spp., Balantidium spp., Loa Loa, Ascaris lumbricoides, Dirofilaria immitis, and Toxoplasma ssp., e.g. Toxoplasma gondii.

A fungal pathogen may be derived from a fungus (including yeast) selected from, but not limited to, the genera Candida spp., (e.g. C.albicans), Epidermophyton spp., Exophiala spp., Microsporum spp., Trichophyton spp., (e.g. T. rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremoniwn spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphiwn spp., Leptosphaeria spp., Malassezia spp. (e.g Malassezia Furfur), Mucor spp., Neotestudina spp., Nocardia spp., Nocardiopsis spp., Paecilomyces spp., Phoma spp., Piedraia spp., Pneunwcystis spp., Pseudallescheria spp., Pyrenochaeta spp., Rhizoinucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., and Wangiella spp. A fungal pathogen may be derived from a fungus (including yeast) selected from, but not limited to, Candida parapsilosis, Aspergillus niger, or a combination thereof.

A fungal, bacterial, or viral infection may be a systemic, topical, subcutaneous, cutaneous or mucosal infection. Topical fungal infections of nails and skin are generally caused by detinatophytes although some non-dermatophytes such as yeast can also cause skin infections. A dermatophyte infection may include a Tinea infection for example Tinea barbae (beard), Tinea capitis (head), Tinea corporis (body), Tinea cruris (groin), Tinea faciei (face), Tinea manuum (hand), Tinea pedis (foot) Tinea unguium (nail), Tinea (Pityriasis) versicolor, Tinea incognito or Tinea nigra. An infection may be derived from fungi of the genera Epidermophyton, Microsporum or Trichophyton spp. (e.g., T. rubrum and T. interdigitale). Exemplary dermatophytes can include Epidermophyton floccosum, Microsporum canis, Microsporum audouinii, Microsporum gypseum, Microsporum nanum, Microsporum ferrugineum, Microsporum distortum, Microsporum fulvum, Trichophyton rubrum, Trichophyton tnentagrophytes var. interdigitale, Trichophyton mentagrophytes var. nodulare, Trichophyton tonsurans, Trichophyton soudanese, Trichophyton violaceum, Trichophyton megnini, Trichophyton schoenlenii, Trichophyton gallinae, Trichophyton krajdenii, Trichophyton yaoundei, Trichophyton equinum, Trichophyton erinacei and Trichophyton verrucosum. In some embodiments, a dermatophytic infection can be onychomycosis. The term “onychomycosis” can include, but is not limited to, distal lateral subungual, superficial white, proximal white subungual, secondary dystrophic, primary dystrophic, endonyx, candidal (e.g., onycholysis & chronic mucocutaneous disease) types of onychomycosis and Tinea ungium. Non-dermatophytic fungi associated with onychomycosis can include Aspergillus spp., Cephalosporum spp., Fusarium oxysporum, Scopularis brevicaulis, and Scytalidium spp.

### Kits

Disclosed herein are kits. A kit can comprise a peptide, pharmaceutically acceptable salt thereof, formulation, or formulation described herein. In some aspects, a peptide, formulation, or formulation can be packaged in a container. In some aspects, a kit can further comprise one or more aqueous carriers in a separate container from the peptide or formulation. In some aspects, a kit comprises one aqueous carrier. In some aspects, a kit comprises more than one aqueous carrier. In some aspects, a kit comprises two aqueous carrier. In some aspects, a kit can further comprise a separate container for mixing (e.g., an intravenous bag) an aqueous carrier or aqueous carriers and the peptide or formulation. In some embodiments, the pharmaceutical composition is mixed with one or more aqueous carriers of a kit in a separate container for mixing prior to use. In some aspects, the aqueous carrier in the kit dilutes the concentration of the peptide or formulation to about 0.5 mg/mL, about 1 mg/mL, about 2 mg/mL, about 3 mg/mL, about 5 mg/mL, about 10 mg/mL, about 15 mg/mL, about 20 mg/mL or about 50 mg/mL. In some aspects, a kit can further comprise instructions that direct administration of a unit dose of a peptide or formulation to a subject. In some aspects, a kit can comprise a peptide disclosed herein and instructions for the use thereof.

Methods of making a kit can include placing a peptide, pharmaceutically acceptable salt thereof, formulation, or formulation described herein in a container for packaging. A method can further comprise an inclusion of instructions for use. In some embodiments, instructions for use can direct administration of a unit dose of a peptide or formulation to a subject.

### Terminology

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, the terms “biofilm”, “microbial film”, “microbial biofilm”, “bacterial film”, refers to any film comprising microorganisms and their excretions.

As used herein, the terms “comprising,” “comprise” or “comprised,” and variations thereof, in reference to elements of an item, formulation, apparatus, method, process, system, claim etc. are intended to be open-ended, meaning that the item, formulation, apparatus, method, process, system, claim etc. includes those elements and other elements can be included and still fall within the scope/definition of the described item, formulation, apparatus, method, process, system, claim etc. As used herein, “a” or “an” means one or more. As used herein “another” may mean at least a second or more.

As used herein, the term “object” refers to any object with a surface. Some embodiments in the present disclosure may be applied to the surface of an object to prevent or to treat microbial biofilm. In some embodiments, the object can a solid object, a liquid object, a hard object, a soft object, a metallic object, a polymeric object, a ceramic object, a composite object, a biological object, members of the animal kingdom, a human being, a biological transplant object, a replaced joint, or any other object with a surface on which some of the disclosed methods and the formulations can be applied.

As used herein, the terms “patient” or “subject” generally refer to any individual that has, may have, or may be suspected of having a disease condition (e.g., a bacterial infection). In some embodiments, the bacterial infection may be caused by surgeries, physical wounds, etc. The subject may be an animal. The animal can be a mammal, such as a human, non-human primate, a rodent such as a mouse or rat, a dog, a cat, pig, sheep, or rabbit. Animals can be fish, reptiles, or others. Animals can be neonatal, infant, adolescent, or adult animals. The subject may be a living organism. The subject may be a human. Humans can be greater than or equal to 1, 2, 5, 10, 20, 30, 40, 50, 60, 65, 70, 75, 80 or more years of age. A human may be from about 18 to about 90 years of age. A human may be from about 18 to about 30 years of age. A human may be from about 30 to about 50 years of age. A human may be from about 50 to about 90 years of age. The subject may have one or more risk factors of a condition and be asymptomatic. The subject may be asymptomatic of a condition. The subject may have one or more risk factors for a condition. The subject may be symptomatic for a condition. The subject may be symptomatic for a condition and have one or more risk factors of the condition. The subject may have or be suspected of having a disease, such as an infection. The subject may be a patient being treated for a disease, such as an infection. The subject may be predisposed to a risk of developing a disease such as a bacterial infection. The subject may be in remission from a disease, such as a bacterial infection. The subject may not have a bacterial infection. The subject may be healthy.

As used herein, a “pharmaceutically acceptable excipient”, “aqueous carrier” or “pharmaceutically acceptable aqueous carrier” refer to solvents or dispersion media, and the like, that are physiologically compatible and known to those skilled in the art. Examples of pharmaceutically acceptable carriers include one or more of water, saline, phosphate buffered saline, dextrose, glycerol, ethanol, and the like, as well as combinations thereof. Pharmaceutically acceptable carriers may further comprise minor amounts of auxiliary substances such as wetting or emulsifying agents, preservatives or buffers, which enhance the shelf life or effectiveness of the active agent.

As used herein, a “effective amount” of an active agent can refer to an amount that is effective to achieve a desired result. An effective amount of a given active agent can vary with respect to factors such as the type and severity of the disorder or disease being treated and the age, gender, and weight of the patient.

The term “homology” can refer to a % identity of a polypeptide to a reference polypeptide. As a practical matter, whether any particular polypeptide can be at least 50%, 60%, 70%, 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters can be set such that the percentage of identity is calculated over the full length of the reference amino acid sequence and that gaps in homology of up to 5% of the total number of amino acid residues in the reference sequence are allowed.

For example, in a specific embodiment the identity between a reference sequence (query sequence, i.e., a sequence of the present invention) and a subject sequence, also referred to as a global sequence alignment, may be determined using the FASTDB computer program based on the algorithm of Brutlag et al. (Comp. App. Biosci. 6:237-245 (1990)). In some embodiments, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some embodiments, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 amino acid residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

The terms “co-administration”, “administered in combination with” and their grammatical equivalents or the like, as used herein, can encompass administration of selected therapeutic agents to a subject, and can include treatment regimens in which agents are administered by the same or different route of administration or at the same or different times. In some embodiments, a peptide disclosed herein can be co-administered with other agents. These terms can encompass administration of two or more agents to a subject so that both agents and/or their metabolites are present in the subject at the same time. They can include simultaneous administration, administration at different times, and/or administration in a formulation in which both agents are present. Thus, in some embodiments, a peptide and an additional agent(s) can be administered in a single formulation. In some embodiments, a peptide and an additional agent(s) can be admixed in the formulation. In some embodiments, a same peptide or agent can be administered via a combination of different routes of administration. In some embodiments, each agent administered can be in a therapeutically effective amount.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Exemplary Peptide Formulations

This example illustrates the exemplary peptide formulations.

Peptide Formulation A: 282.59 mg of SEQ ID NO: 1 was added to 200 mL of 0.9% saline solution. 1N sodium hydroxide or 1N hydrochloric acid was added into peptide formulation A to adjust the pH to 5.0±0.2. The concentration of the peptide was 1.0 mg/mL.

Peptide Formulation B: 282.44 mg of SEQ ID NO: 1 was added to 200 mL of glycine buffer (7.5 mg/mL, 0.1M). 1N sodium hydroxide or 1N hydrochloric acid was added into peptide formulation B to adjust the pH to 7.4±0.2. The concentration of the peptide was 1.0 mg/mL.

Peptide Formulation C: 282.53 mg of SEQ ID NO: 1 was added to 200 mL of tromethamine buffer (2.422 mg/mL, 20 mM). 1N sodium hydroxide or 1N hydrochloric acid was added into peptide formulation C to adjust the pH to 7.4±0.2. The concentration of the peptide was 1.0 mg/mL.

Peptide Formulation D: 282.5 mg of SEQ ID NO: 1 is added to 200 mL of 0.9% saline solution. Acetic acid is added into peptide formulation D to adjust the pH to 5.0±0.2. The concentration of the peptide is 1 mg/mL.

Peptide Formulation E: 847.5 mg of SEQ ID NO: 1 is added to 200 mL of 0.9% saline solution. Acetic acid is added into peptide formulation E to adjust the pH to 5.0±0.2. The concentration of the peptide is 3 mg/mL.

Peptide Formulation F: 2.825 g of SEQ ID NO: 1 is added to 200 mL of 0.9% saline solution. Acetic acid is added into peptide formulation F to adjust the pH to 5.0±0.2. The concentration of the peptide is 10 mg/mL.

Peptide Formulation G: 11.3 g of SEQ ID NO: 1 is added to 200 mL of 0.9% saline solution. Acetic acid is added into peptide formulation G to adjust the pH to 5.0±0.2. The concentration of the peptide is 40 mg/mL.

Peptide Formulation H: 18.363 g of SEQ ID NO: 1 is added to 200 mL of 0.9% saline solution. Acetic acid is added into peptide formulation H to adjust the pH to 5.0±0.2. The concentration of the peptide is 65 mg/mL.

Peptide Formulation I: 25.425 g of SEQ ID NO: 1 is added to 200 mL of 0.9% saline solution. Acetic acid is added into peptide formulation Ito adjust the pH to 5.0±0.2. The concentration of the peptide is 90 mg/mL.

### Example 2: Stability of Peptide Formulations

This example illustrates the stability of peptide formulations tested after storage and different temperature.

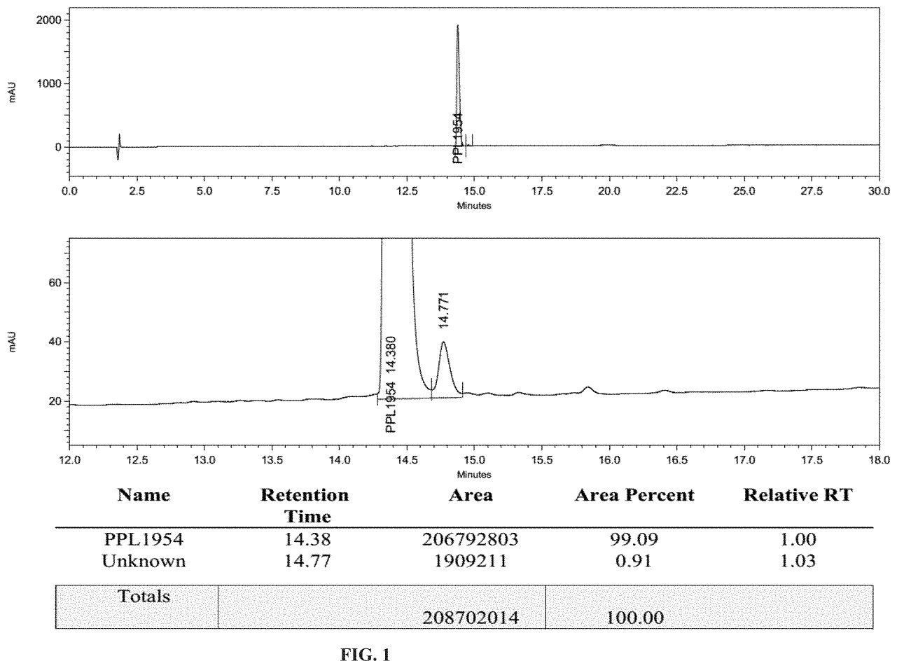

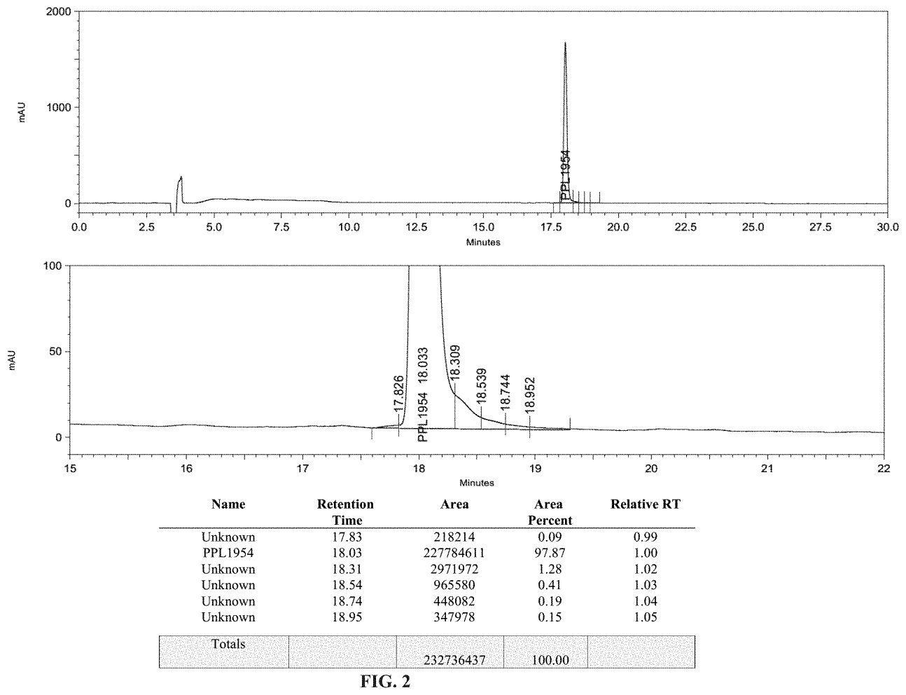

A solid form of SEQ ID NO: 1 from Batch 2K18005 was dissolved and assayed. FIG. 1 depict the chromatograph of SEQ ID NO:1 at 1.0 mg/mL. The retention time at 14.38 and relative retention time at 1.00 (RTT) corresponded to SEQ ID NO: 1. The area percent of the SEQ ID NO: 1 was 99.09. The retention time at 14.77 and RTT at 1.03 corresponded to an impurity. The area percent of the impurity was 0.91. FIG. 1 depicts SEQ ID NO: 1 at time (t)=0 for a baseline prior to storage. SEQ ID NO: 1 is stable after 3 years of storage at −20° C. as a dry powder in a glass vial. FIG. 2 depicts the HPLC chromatograph after 3 years of storage SEQ ID NO: 1 in solid form at −20° C. (Batch 2K18005). The retention time at 18.03 and relative retention time at 1.00 (RTT) corresponded to SEQ ID NO: 1. The area percent of the SEQ ID NO: 1 was 97.87. The retention times at 18.83 (RTT at 0.99), 18.31 (RTT at 1.02), 18.54 (RTT at 1.03), 18.74 (RTT at 1.04), 18.95 (RTT at 1.05) corresponded to a plurality of impurities with area percentages of 0.09, 1.28, 0.41, 0.19, and 0.15, respectively.

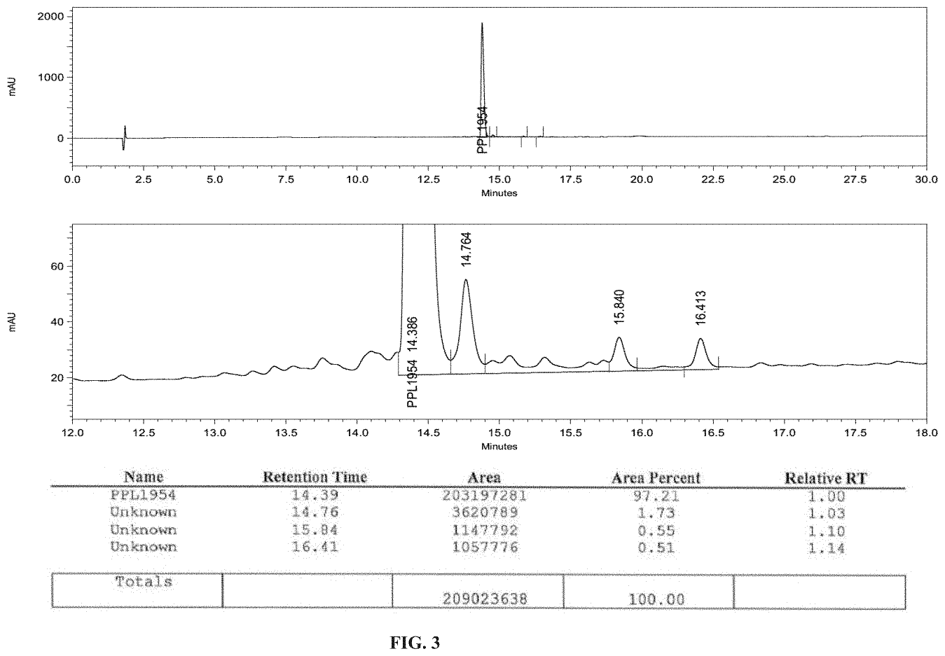

A solid form of SEQ ID NO: 1 from Batch AMF110 was dissolved and assayed. FIG. 3 depict the chromatograph of 1.0 mg/mL SEQ ID NO:1 at time (t)=0 for a baseline prior to storage. The retention time at 14.39 and relative retention time at 1.00 (RTT) corresponded to SEQ ID NO: 1. The area percent of the SEQ ID NO: 1 was 97.21. The retention times at 14.76 (RTT at 1.03), 15.84 (RTT at 1.10), and 16.41 (RTT at 1.14) corresponded to a plurality of impurities with area percentages of 1.73, 0.55, and 0.51, respectively.

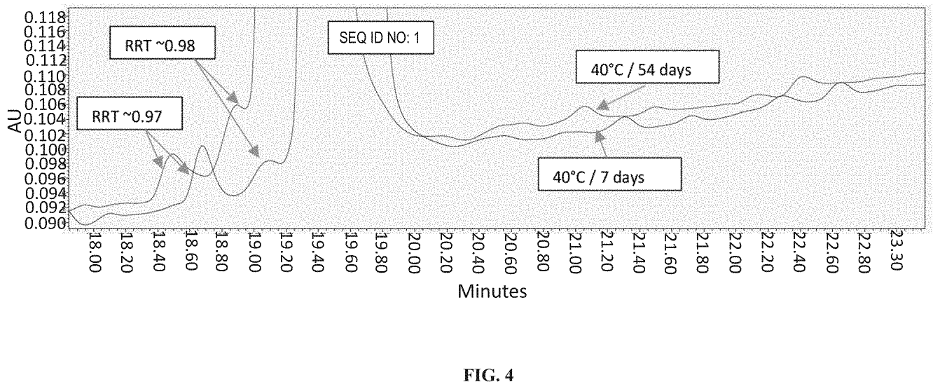

Enhanced stability was shown of the peptide formulation after 54 days of storage at 40° C. as a liquid solution with a concentration of 5 mg/mL of SEQ ID NO: 1 at pH of 5.0. FIG. 4 depict the overlaid chromatograph of SEQ ID NO: 1 after 7 days at 40° C. (bottom graph) and after 54 days at 40° C. (top graph). An increase in the level of impurity at RRT 0.98 was shown, from 0.4% to 1.1%. Data compared to the frozen samples at −20° C. is shown in Table 2.

TABLE 2
Summary of analytical results of stored SEQ ID NO: 1.

| | SEQ ID | | Main | Area % of |
|---|---|---|---|---|
| | NO: 1 | | peak SEQ | concurrent |
| | content | Solution | ID NO: | analytical |
| Sample | (mg/mL) | pH | 1 area % | standard |
| 7 days/−20° C. | 5.11 | 4.95 | 97.2 | 97.1 ± 0.4 |
| 14 days/−20° C. | 5.08 | 4.97 | 95.4 | 95.5 ± 0.5 |
| 54 days/−20° C. | 5.09 | 4.95 | 96.9 | 96.8 ± 0.8 |
| 7 days/40° C. | 5.05, 5.11 | 4.98, 5.02 | 97.2, 97.1 | 97.1 ± 0.4 |
| 14 days/40° C. | 5.05, 5.01 | 5.01, 5.00 | 96.2, 94.7 | 95.5 ± 0.5 |
| 54 days/40° C. | 4.99 | 4.96 | 95.6 | 96.8 ± 0.8 |

### Example 3: Stability of Peptide Formulations

This example illustrates the stability of peptide formulations tested after storage with different pH and buffers.

### Experimental Details

SEQ ID NO: 1 in is solid form was white to off-white powder with a solubility of above 100 mg/mL in water at room temperature. SEQ ID NO: 1 was stable at frozen temperatures when protected in light. The pKa was approximately 12.48 for SEQ ID NO: 1 and was approximately 3.4 kDa (˜3399.19 g/mol).

The work focused on peptide formulation development of SEQ ID NO: 1 dissolved in 0.9% saline for pH of 5.0 and dissolved in phosphate buffered saline, Tris buffer and glycine buffers at pH of 7.4. The peptide formulations were placed on stability at inverted conditions of −20° C., 5° C., and room temperature. The concentration of the peptide formulation was maintained at 1 mg/mL.

### Protocol

The saline solution or buffer solutions were prepared in sterile water. The pH of the buffer was recorded. The pH of the buffer or solution was adjusted to the target pH with either 1 N HCl or 1N NaOH. The peptide was dissolved in the buffer or saline. The pH of the dissolved peptide in saline or buffer was adjusted to the target pH with either 1 N HCl or 1N NaOH. The volume was adjusted to 200 mL, additional buffer solution was added into the peptide/buffer formulation and additional saline solution was added into the peptide/saline solution. The solution was filtered through a 0.22 p.m membrane filter. Aliquots of 2 mL of the peptide formulation was placed into 13 mm, 2mL amber glass vials. A stopper was used to enclose the glass vial and crimped with an aluminum seal. The vials were stored under inverted conditions of −20° C., 5° C., or room temperature. The experimental details are shown in Table 3 through Table 8.

TABLE 3
Formulation Details of Peptide Formulations (1 mg/mL)

| | 0.9% saline, | Phosphate Buffer | Tromethamine, | Glycine, |
|---|---|---|---|---|
| | pH 5.0 | Saline (PBS), pH 7.4 | pH 7.4 | pH 7.4 |
| Protocol # | FDL2151.00 | FDL2150.00 | FDL2152.00 | FDL2153.00 |
| Network # | 09620-002-001 | 09620-003-001 | 09620-004-001 | 09620-005-001 |
| Batch # | 9620-2-2 | 9620-3-2 | 9020-4-2 | 920-5-2 |

| | |
|---|---|
| Batch size: | 200 mL |
| Fill Volume | 2 mL |
| API Lot | AMF19 |
| Date of | Oct. 30, 2020 |
| Manufacturing | |

TABLE 4
Quantitative Formulation of SEQ ID NO:
1 in 0.1M Glycine Buffer (1 mg/mL)

| | | Qty/mL | Amount | Amount |
|---|---|---|---|---|
| | | (1 mg/mL | Required for | Weighed |
| Material | Function | Formulation) | 200 mL (mg) | (mg) |
| SEQ ID NO: 1 | Active | 1 mg | 282.1 mg | 282.44 mg* |
| Glycine | Buffer | 7.5 mg | 1500.0 mg | 1503.57 mg |

| Sodium | pH | pH Adjustment (7.4 ± 0.2) | |
|---|---|---|---|
| Hydroxide, 1N | Adjust | | |
| Hydrochloric | pH | | |
| Acid, 1N | Adjust | | |
| Water for | Solvent | Q.S. to 1 mL | Q.S. to 200 mL |
| Injection | | | |

TABLE 5
Quantitative Formulation of SEQ ID NO:
1 in 20 mM Tromethamine Buffer (1 mg/mL)

| | | Qty/mL | Amount | Amount |
|---|---|---|---|---|
| | | (1 mg/mL | Required for | Weighed |
| Material | Function | Formulation) | 200 mL (mg) | (mg) |
| SEQ ID NO: 1 | Active | 1 mg | 282.1 mg | 282.53 mg* |
| Tromethamine | Buffer | 2.422 mg | 484.4 mg | 485.35 mg |

| Sodium | pH | pH Adjustment (7.4 ± 0.2) | |
|---|---|---|---|
| Hydroxide, 1N | Adjust | | |
| Hydrochloric | pH | | |
| Acid, 1N | Adjust | | |
| Water for | Solvent | Q.S. to 1 mL | Q.S. to 200 mL |
| Injection | | | |

TABLE 6
Quantitative Formulation of SEQ ID NO:
1 in Phosphate Buffered Saline (1 mg/mL)

| | | Qty/mL | Amount | Amount |
|---|---|---|---|---|
| | | (1 mg/mL | Required for | Weighed |
| Material | Function | Formulation) | 200 mL (mg) | (mg) |
| SEQ ID NO: 1 | Active | 1 mg | 282.1 mg | 282.59 mg* |
| Phosphate | Buffer | See Table 4 | NA | NA |
| Buffered Saline | | Below for | | |
| | | Preparation¹ | | |

| Sodium | pH | pH Adjustment (7.4 ± 0.2) | |
|---|---|---|---|
| Hydroxide, 1N | Adjust | | |
| Hydrochloric | pH | | |
| Acid, 1N | Adjust | | |
| Water for | Solvent | Q.S. to 1 mL | Q.S. to 200 mL |
| Injection | | | |

TABLE 7
Quantitative Formulation of SEQ ID NO: 1 in Dulbecco’s
Phosphate Buffered Saline, pH 7.4 ± (1 mg/mL)

| | Concentration |
|---|---|
| Component | (mg/mL) |
| Potassium Chloride (KCl) | 0.20 |
| Monobasic Potassium Phosphate (KH₂PO₄) | 0.20 |
| Sodium Chloride (NaCl) | 8.00 |
| Sodium Phosphate Dibasic, Heptahydrate | 2.16 |
| (Na₂HPO₄•7H₂O) | |
| Sodium Hydroxide | pH Adjustment |
| Hydrochloric Acid | (7.4 ± 0.2) |
| Water for Injection | Q.S. to 1 mL |

TABLE 8
Quantitative Formulation of SEQ ID NO: 1 in 0.9% Saline (1 mg/mL)

| | | | Amount | |
|---|---|---|---|---|
| | | Qty/mL | Required | Amount |
| | | (1 mg/mL | for 200 mL | Weighed |
| Material | Function | Formulation) | (mg) | (mg) |
| SEQ ID NO: 1 | Active | 1 mg | 282.1 mg | 282.47 mg* |
| Sodium Chloride | Tonicity | 9 mg | 1800.0 mg | 1801.31 mg |
| | Agent | | | |

| Sodium | pH | pH Adjustment (5.0 ± 0.2) | |
|---|---|---|---|
| Hydroxide, 1N | Adjust | | |
| Hydrochloric | pH | | |
| Acid, 1N | Adjust | | |
| Water for Injection | Solvent | Q.S. to 1 mL | Q.S. to 200 mL |

TABLE 9
Initial and Stability Results of Purity by area % and Total Impurities by area % for SEQ ID NO: 1 Batches

| | | | | | 1 Month Results | | | 2 Month Results | | | 3 Month Results | | |
|---|---|---|---|---|---|---|---|---|---|---|---|---|---|
| | | | Initial | | | | % | | | % | | | % |
| | | | Results | | | | Total | | | Total | | | Total |
| | | Storage | Purity | % | | | Im- | | | Im- | Purity | | Im- |
| Sam- | | Con- | by | Total | Purity | % Individual | purities | Purity | % Individual | purities | by | % Individual | purities |
| ple | Form- | dition | area | Im- | by area | impurities | by | by area | impurities | by area | area | impurities | by |
| Lot | ulation | (° C.) | % | purities | % | at RRT | area % | % | at RRT | % | % | at RRT | area % |
| 9620- | 0.9% | 5 | 100.0 | 0.0 | 98.9 | RRT 1.014: 1.1% | 1.1 | 98.1 | RRT~ 0.771: 0.7% | 1.9 | 98.7 | RRT~ 1.016: 1.3% | 1.3 |
| 2-2 | Saline, | | | | | | | | RRT~ 1.015: 1.2% | | | | |
| | pH 5.0 | Ambient | | | 98.8 | RRT 1.014: 1.2% | 1.2 | 98.8 | RRT~ 1.014: 1.2% | 1.2 | 98.6 | RRT~ 1.016: 1.4% | 1.4 |
| | | −20 | | | 98.9 | RRT 1.014: 1.1% | 1.1 | 98.3 | RRT~0.773: 0.5% | 1.7 | 98.7 | RRT~ 1.016: 1.3% | 1.3 |
| | | | | | | | | | RRT~ 1.014: 1.2% | | | | |
| 9620- | PBS, | 5 | 100.0 | 0.0 | 98.8 | RRT 1.014: 1.2% | 1.2 | 98.2 | RRT~ 1.014: 1.3% | 1.8 | 97.3 | RRT~ 1.014: 1.5% | 2.7 |
| 3-2 | pH 7.4 | | | | | | | | RRT~ 1.084: 0.5% | | | RRT~ 1.090: 0.8% | |
| | | | | | | | | | | | | RRT~ 1.260: 0.5% | |
| | | Ambient | | | 98.7 | RRT 1.014: 1.3% | 1.3 | 97.6 | RRT~ 1.014: 1.4% | 2.4 | 96.5 | RRT~ 1.016: 1.7% | 3.5 |
| | | | | | | | | | RRT~ 1.060: 0.5% | | | RRT~ 1.062: 0.7% | |
| | | | | | | | | | RRT~ 1.085: 0.5% | | | RRT~ 1.076: 0.5% | |
| | | | | | | | | | | | | RRT~ 1.088: 0.7% | |
| | | −20 | | | 98.8 | RRT 1.014: 1.2% | 1.2 | 97.7 | RRT~0.776: 0.5% | 2.3 | 97.9 | RRT~ 1.016: 1.3% | 2.1 |
| | | | | | | | | | RRT~ 1.014: 1.3% | | | RRT~ 1.088: 0.8% | |
| | | | | | | | | | RRT~ 1.085: 0.5% | | | | |
| 9620- | Tro- | 5 | 100.0 | 0.0 | 98.9 | RRT 1.014: 1.1% | 1.1 | 98.7 | RRT~ 1.014: 1.3% | 1.3 | 98.7 | RRT~ 1.016: 1.3% | 1.3 |
| 4-2 | meth- | Ambient | | | 98.8 | RRT 1.014: 1.2% | 1.2 | 98.6 | RRT~ 1.014: 1.4% | 1.4 | 96.8 | RRT~0.981: 0.6% | 3.2 |
| | amine, | | | | | | | | | | | RRT~ 1.016: 1.6% | |
| | pH 7.4 | | | | | | | | | | | RRT~ 1.077: 0.5% | |
| | | | | | | | | | | | | RRT~ 1.088: 0.5% | |
| | | −20 | | | 98.9 | RRT 1.014: 1.1% | 1.1 | 98.2 | RRT~0.771: 0.6% | 1.8 | 98.7 | RRT~ 1.016: 1.3% | 1.3 |
| | | | | | | | | | RRT~ 1.014: 1.2% | | | | |
| 9620- | Glycine, | 5 | 100.0 | 0.0 | 98.9 | RRT 1.014: 1.1% | 1.1 | 98.8 | RRT~ 1.014: 1.2% | 1.2 | 97.6 | RRT~ 1.017: 1.4% | 2.4 |
| 5-2 | pH 7.4 | | | | | | | | | | | RRT~ 1.088: 0.5% | |
| | | | | | | | | | | | | RRT~ 1.261: 0.5% | |
| | | Ambient | | | 98.8 | RRT 1.014: 1.2% | 1.2 | 97.7 | RRT~0.979: 0.5% | 2.3 | 96.5 | RRT~0.981: 0.7% | 3.5 |
| | | | | | | | | | RRT~ 1.014: 1.3% | | | RRT~ 1.017: 1.5% | |
| | | | | | | | | | RRT~ 1.257: 0.5% | | | RRT~ 1.247: 0.6% | |
| | | | | | | | | | | | | RRT~ 1.262: 0.8% | |
| | | −20 | | | 98.9 | RRT 1.014: 1.1% | 1.1 | 98.4 | RRT~0.774: 0.5% | 1.6 | 98.2 | RRT~ 1.017: 1.3% | 1.8 |
| | | | | | | | | | RRT~ 1.014: 1.1% | | | RRT~ 1.262: 0.5% | |

TABLE 10
Initial and Stability UV Assay and Total Impurities by HPLC Data for SEQ ID NO: 1 Batches

| | | | Initial Results | | 1 Month Results | | 2 Month Results | | 3 Month Results | |
|---|---|---|---|---|---|---|---|---|---|---|
| | | | | Total | | Total | | Total | | Total |
| | | Storage | Assay | Impurities | Assay | Impurities | Assay | Impurities | Assay | Impurities |
| Sample | | Condition | by | by area % | by | by area % | by | by area % | by | by area % |
| Lot | Formulation | (° C.) | UV | by HPLC | UV | by HPLC | UV | by HPLC | UV | by HPLC |
| 9620-2-2 | 0.9% Saline, | Ambient | 91.0 | 0.0 | 94.1 | 1.2 | 99.9 | 1.2 | 89.7 | 1.4 |
| | pH 5.0 | 5 | | 0.0 | 92.4 | 1.1 | 98.4 | 1.9 | 91.3 | 1.3 |
| | | −20 | | 0.0 | 94.3 | 1.1 | 98.0 | 1.7 | 53.7** | 1.3 |
| 9620-3-2 | PBS, pH 7.4 | Ambient | 92.5 | 0.0 | 90.9 | 1.3 | 92.4* | 2.4 | 90.4 | 3.5 |
| | | 5 | | 0.0 | 93.3 | 1.2 | 92.8* | 1.8 | 89.3 | 2.7 |
| | | −20 | | 0.0 | 92.4 | 1.2 | 94.3* | 2.3 | 89.5 | 2.1 |
| 9620-4-2 | Tromethamine, | Ambient | 91.4 | 0.0 | 95.0 | 1.2 | 101.7 | 1.4 | 92.1 | 3.2 |
| | pH 7.4 | 5 | | 0.0 | 95.6 | 1.1 | 104.3 | 1.3 | 96.7 | 1.3 |
| | | −20 | | 0.0 | 91.9 | 1.1 | 97.7 | 1.8 | 90.8 | 1.3 |
| 9620-5-2 | Glycine, | Ambient | 96.2 | 0.0 | 100.1 | 1.2 | 102.3 | 2.3 | 97.0 | 3.5 |
| | pH 7.4 | 5 | | 0.0 | 100.6 | 1.1 | 100.9 | 1.2 | 97.2 | 2.4 |
| | | −20 | | 0.0 | 99.4 | 1.1 | 102.6 | 1.6 | 95.3 | 1.8 |

*Result is average of 3 measurements,
**Result not representative of sample; due to limited sample, 2″d sample vial from pH test was combined with original limited sample.

TABLE 11
Initial and Stability Osmolarity, pH, and Particulate Matter Data for SEQ ID NO: 1 Batches

| | | 9620-2-2 | | | | 9620-3-2 | | | | 9620-4-2 | | | | 9620-5-2 | | | |
|---|---|---|---|---|---|---|---|---|---|---|---|---|---|---|---|---|---|
| | Storage Condition | (0.9% Saline, pH 5.0) | | | | (PBS, pH 7.4) | | | | (Tromethamine, pH 7.4) | | | | (Glycine, pH 7.4) | | | |
| Test | (° C.) | T0 | 1M | 2M | 3M | T0 | 1M | 2M | 3M | T0 | 1M | 2M | 3M | T0 | 1M | 2M | 3M |
| ¹Osmolality | Ambient | 290 | | | | 287 | | | | 36 | | | | 105 | | | |
| (mOsm/kg) | 5 | | | | | | | | | | | | | | | | |
| | −20 | | | | | | | | | | | | | | | | |
| pH | Ambient | 5.2 | 5.0 | 5.0 | 5.0 | 7.4 | 7.4 | 7.4 | 7.4 | 7.5 | 7.4 | 7.4 | 7.3 | 7.5 | 7.3 | 7.3 | 7.3 |
| | 5 | | 4.9 | 4.9 | 4.9 | | 7.4 | 7.4 | 7.3 | | 7.4 | 7.4 | 7.4 | | 7.4 | 7.4 | 7.3 |
| | −20 | | 5.0 | 4.9 | 4.9 | | 7.4 | 7.4 | 7.4 | | 7.3 | 7.4 | 7.2 | | 7.3 | 7.4 | 7.3 |

| | | | | | | | | | | |
|---|---|---|---|---|---|---|---|---|---|---|
| ²Particulate | Ambient | ≥10 pm | 6 | 3 | 33 | 19 | 2 | 1 | 1 | 1 |
| Matter | | ≥25 pm | 0 | 1 | 1 | 1 | 0 | 0 | 0 | 0 |
| | 5 | ≥10 pm | | 3 | | 12 | | 2 | | 1 |
| | | ≥25 pm | | 0 | | 0 | | 0 | | 0 |
| | −20 | ≥10 pm | | 3 | | 21 | | 1 | | 1 |
| | | ≥25 pm | | 1 | | 0 | | 0 | | 0 |

¹Osmolality was tested at initial timepoint only.
²Particulate Matter was tested at initial and 3-month timepoints only.

TABLE 12
Initial and Stability Osmolarity, pH, and Particulate Matter Data for SEQ ID NO: 1 Batches

| | Storage | 9620-2-2 | | | | 9620-3-2 | | | | 9620-4-2 | | | | 9620-5-2 | | | |
|---|---|---|---|---|---|---|---|---|---|---|---|---|---|---|---|---|---|
| | Condition | (0.9% Saline, pH 5.0) | | | | (PBS, pH 7.4) | | | | (Tromethamine, pH 7.4) | | | | (Glycine, pH 7.4) | | | |
| Test | (° C.) | T0 | 1M | 2M | 3M | T0 | 1M | 2M | 3M | T0 | 1M | 2M | 3M | T0 | 1M | 2M | 3M |

| | | | | | |
|---|---|---|---|---|---|
| Appearance | Ambient | Clear, colorless liquid. | Clear, colorless liquid. | Clear, colorless liquid. | Clear, colorless liquid. |
| | 5 | Nothing observed in | Nothing observed in | Nothing observed in | Nothing observed in |
| | −20 | sample when in | sample when in | sample when in | sample when in |
| | | motion. Housed | motion. Housed | motion. Housed | motion. Housed |
| | | in a brown | in a brown | in a brown | in a brown |
| | | glass vial with a | glass vial with a | glass vial with a | glass vial with a |
| | | legible label, metal | legible label, metal | legible label, metal | legible label, metal |
| | | crimp seal, and gray | crimp seal, and gray | crimp seal, and gray | crimp seal, and gray |
| | | rubber stopper. | rubber stopper. | rubber stopper. | rubber stopper. |

TABLE 13
6 Month Stability Data for 0.9% Saline, pH 5.0

| | | | | | Total Impurities | |
|---|---|---|---|---|---|---|
| Stability | | | % Assay | Impurities by | by area % by | Purity by area |
| condition | Appearance | pH | by UV | RRT = 1.03 | HPLC | % by HPLC |
| Ambient | Clear, colorless liquid. Nothing | 4.9 | 100.9 | 0.9 | 0.9 | 99 |
| 5° C. | observed in sample when in motion. | 4.9 | 93.8 | 0.7 | 0.7 | 99 |
| −20° C. | Housed in a brown glass vial with a | 4.9 | 97.8 | 0.8 | 0.8 | 99 |
| | legible label, metal crimp seal, and | | | | | |
| | gray grubber stopper. | | | | | |

### Results

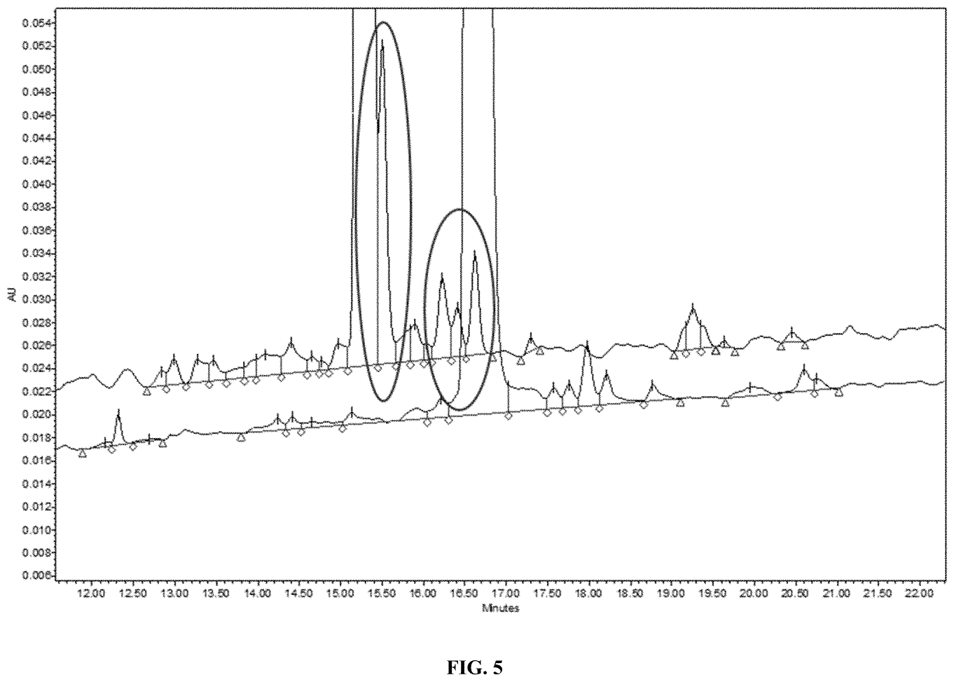

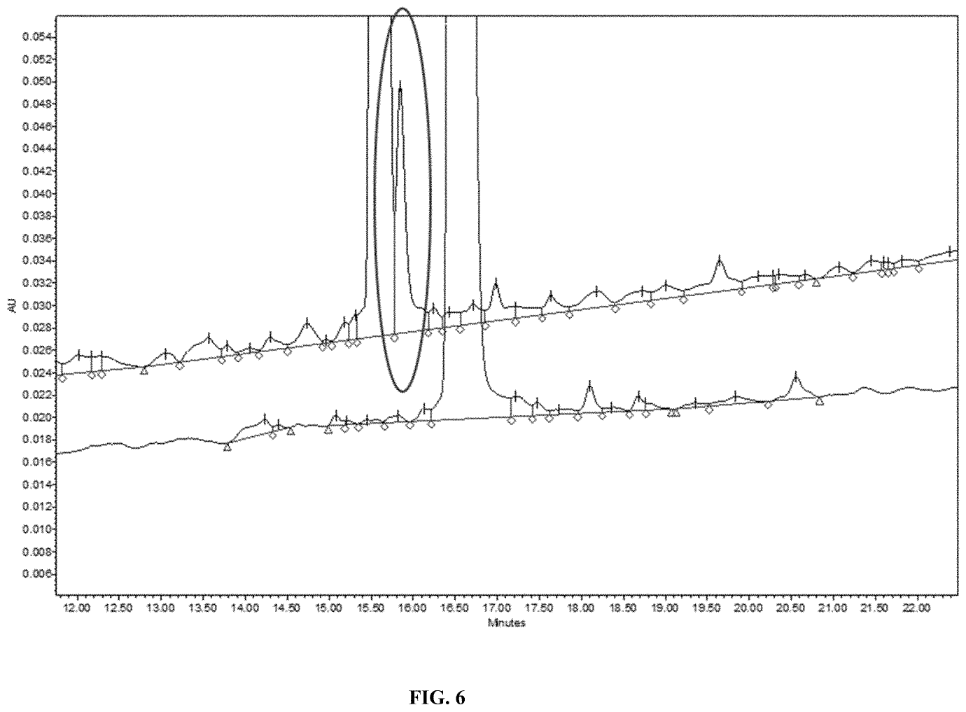

Enhanced stability was shown of the peptide formulation at pH of 5.0 in saline solution. FIG. 5 depict an overlaid HPLC chromatograph of SEQ ID NO: 1 and impurities at an initial time point (0 day) (bottom graph) and after 3 months (top graph). The peptide formulation was stored in PBS buffer at pH of 7.4. Impurity peaks are circled on the 3 months graph. FIG. 6 depict an overlaid HPLC chromatograph of SEQ ID NO: 1 at an initial time point (0 day) (bottom) and after 3 months (top) stored in saline at pH of 5.0. The peptide formulation was stored in a saline solution at pH of 5.0. The single impurity peak was circled on the 3 months graph.

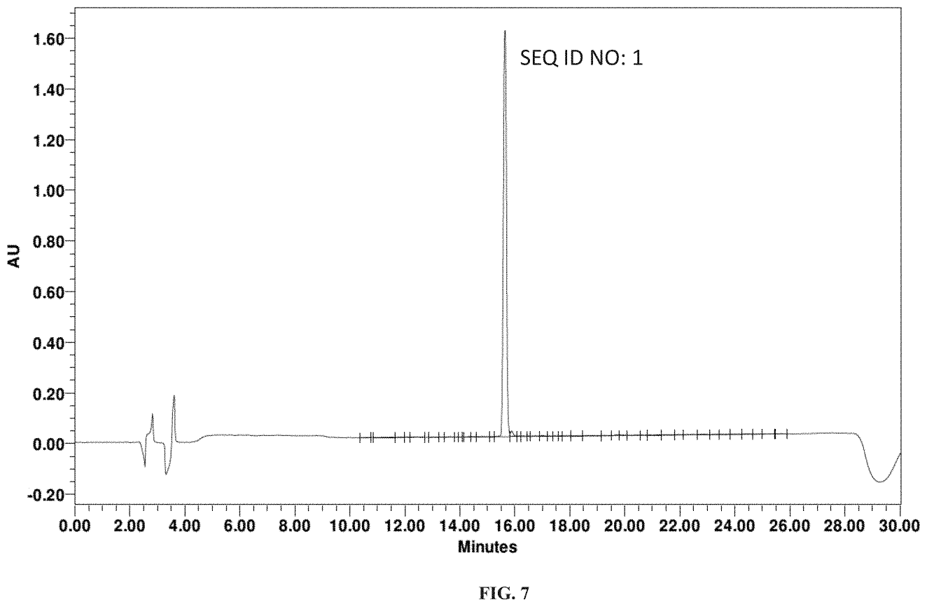

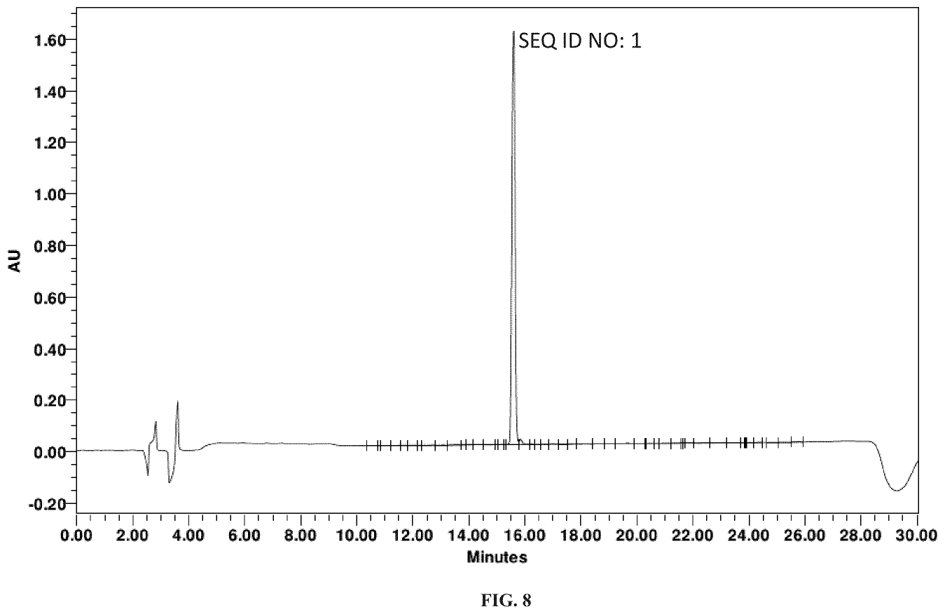

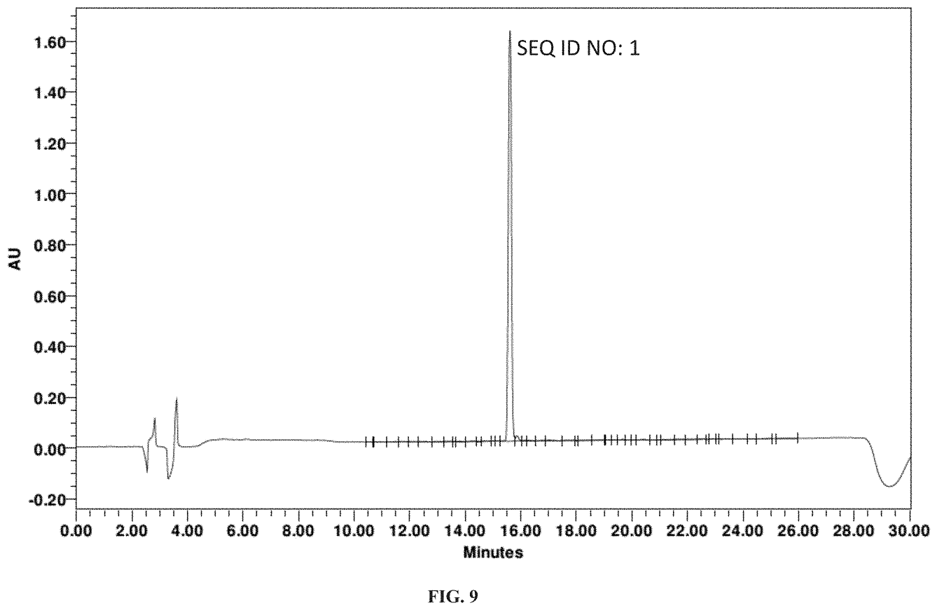

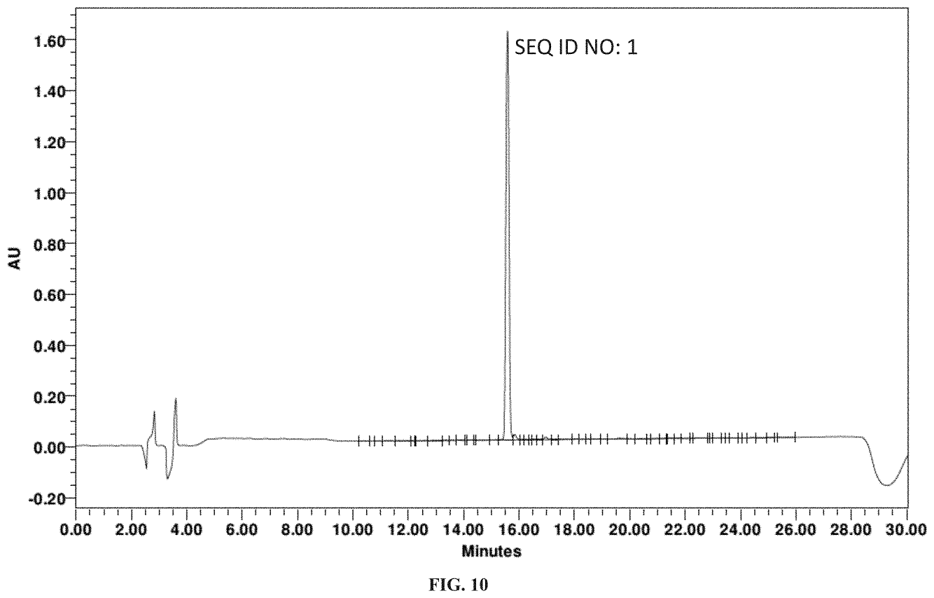

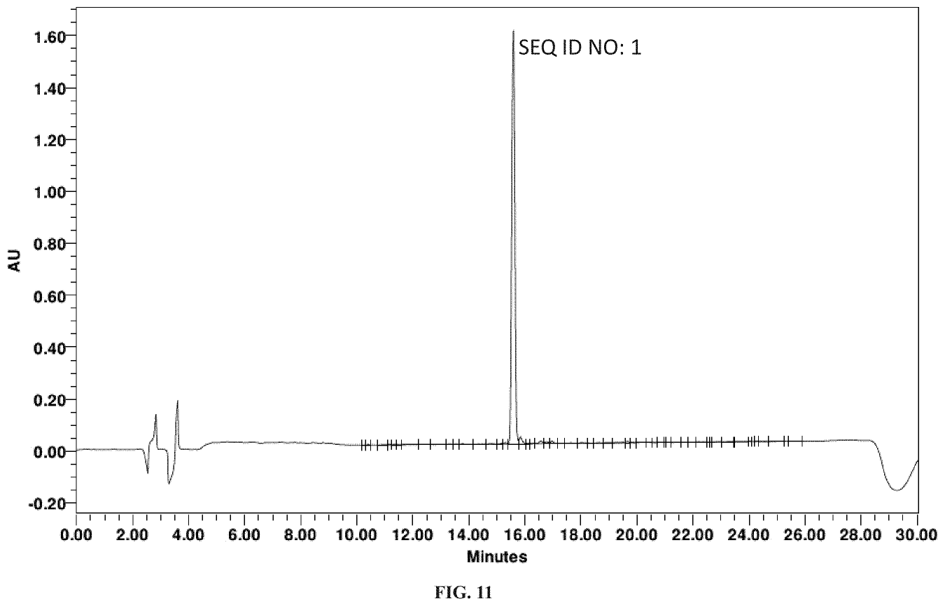

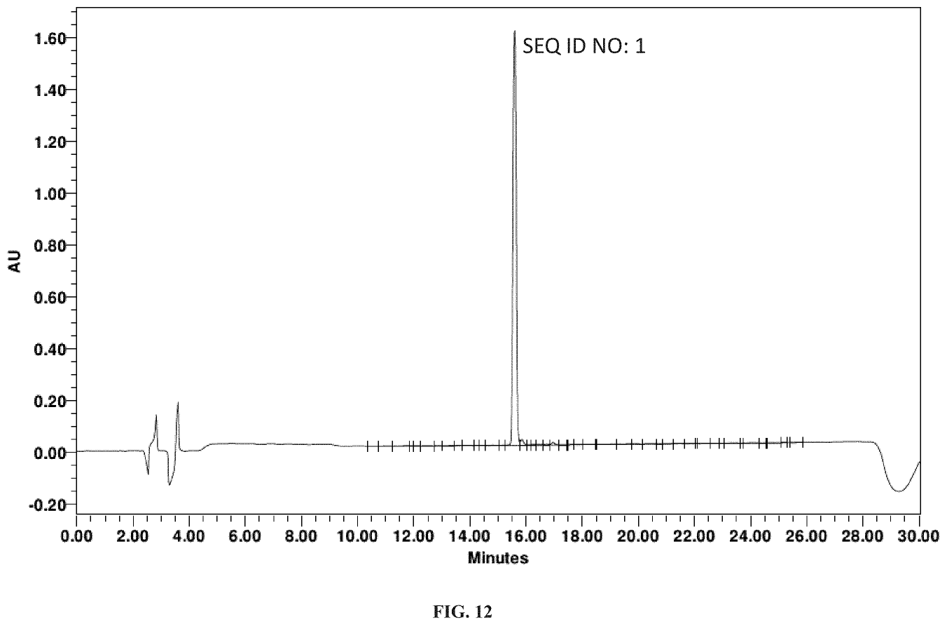

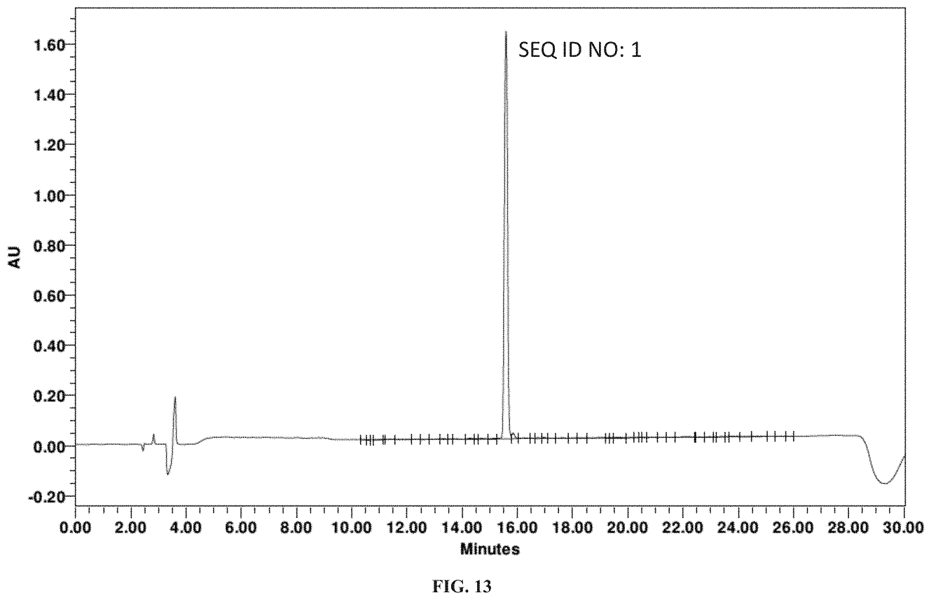

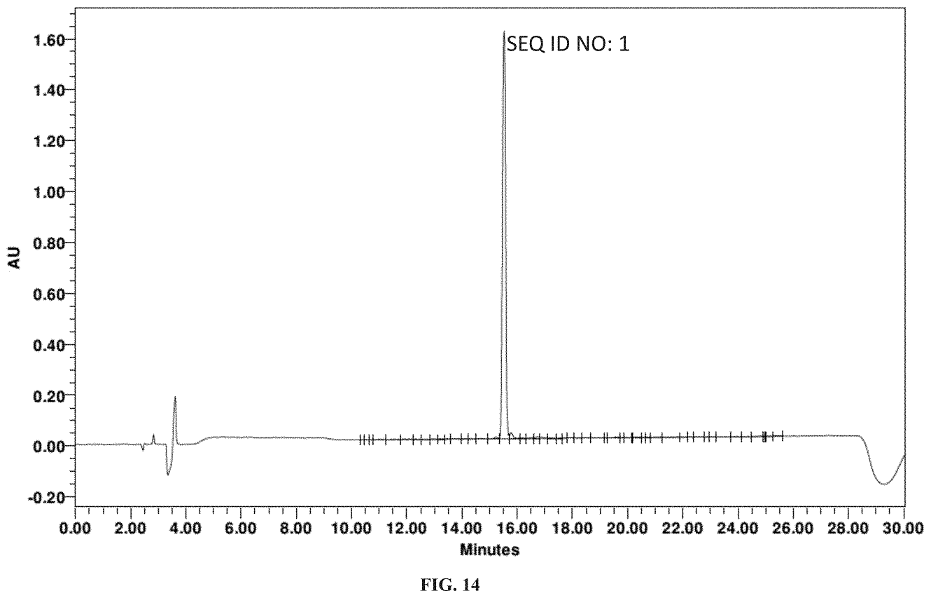

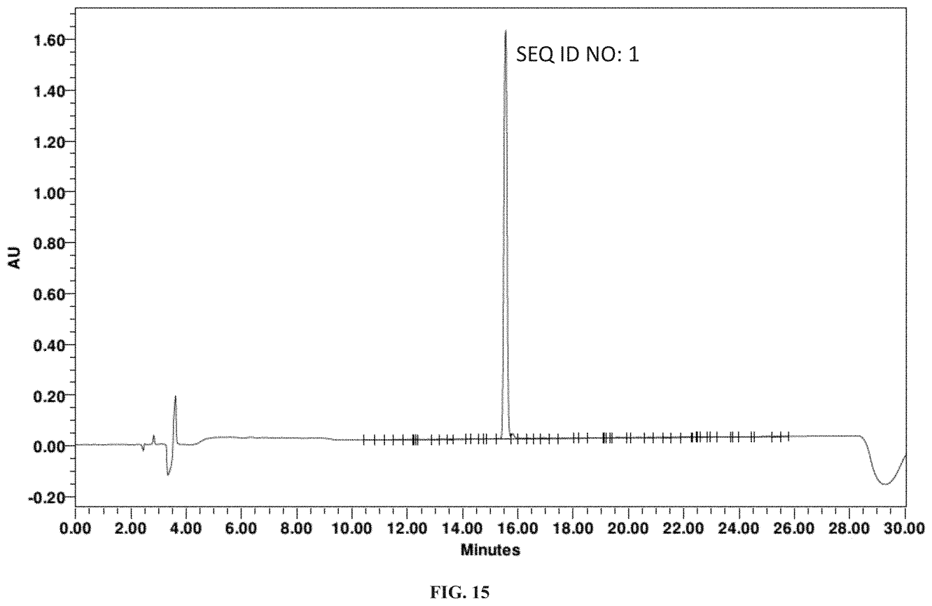

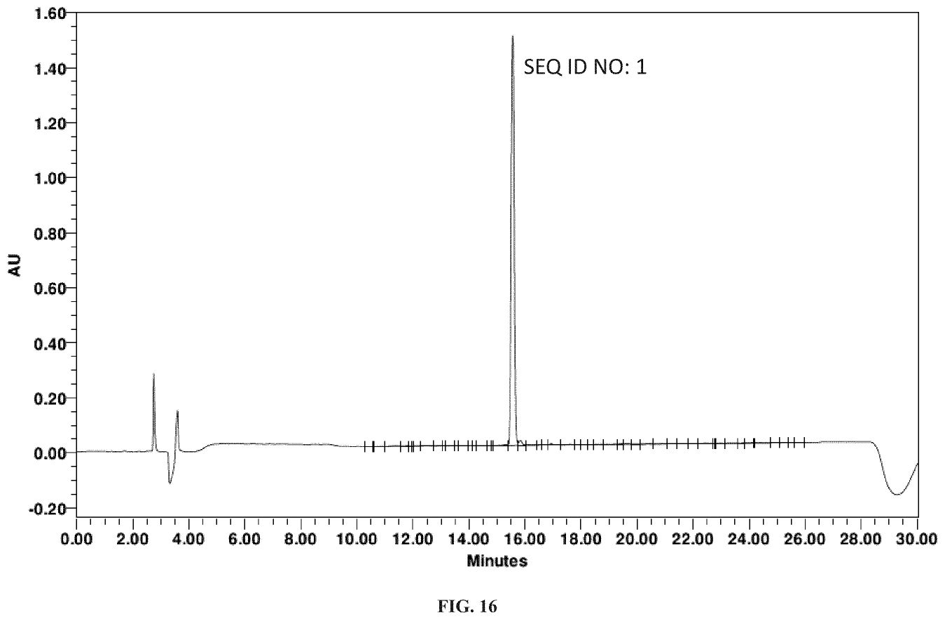

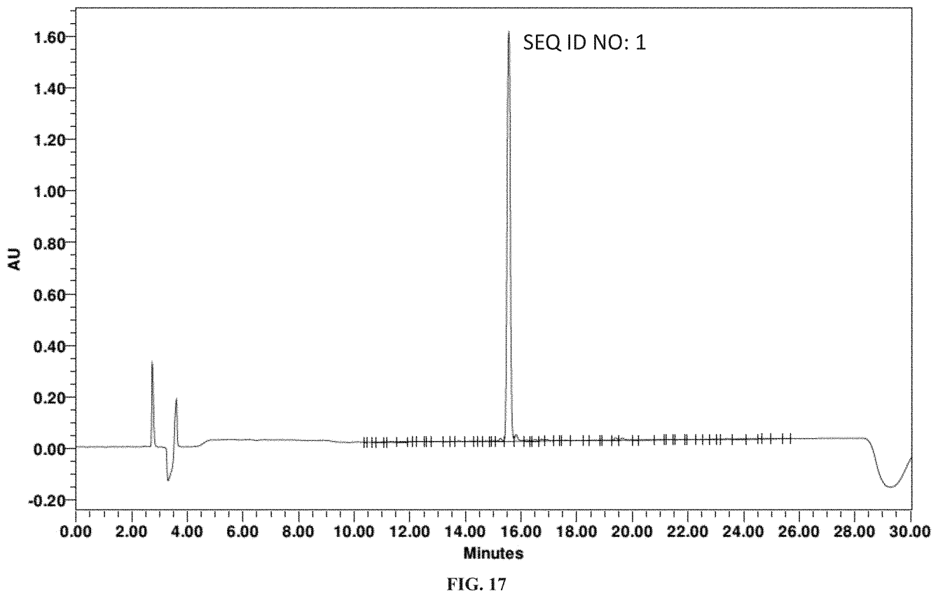

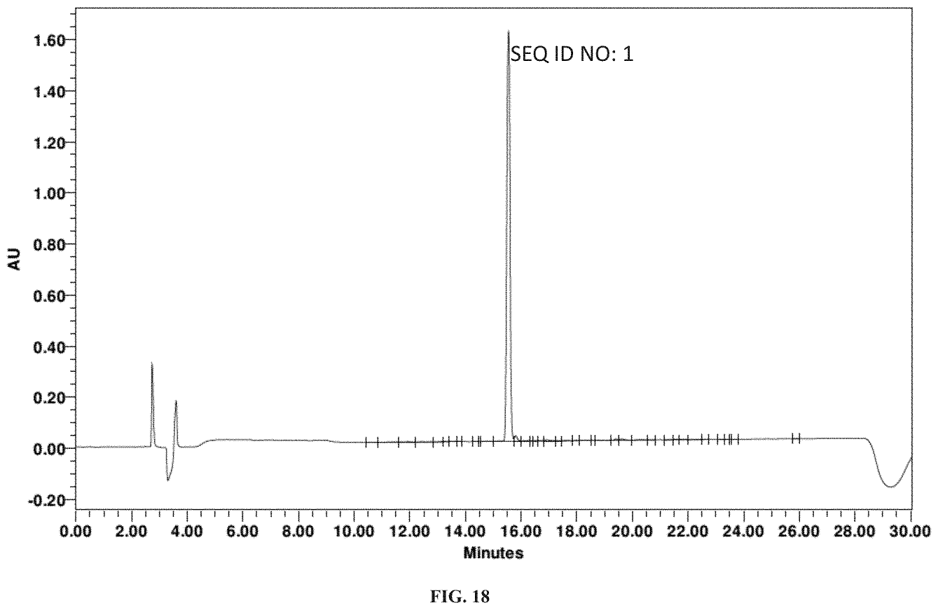

FIG. 7 depicts the HPLC chromatograph of 1.0 mg/mL SEQ ID NO: 1 at after 3 months stored at 5° C. in saline at pH of 5.0. The retention time at 15.639 minutes correspond to SEQ ID NO: 1. FIG. 8 depicts the HPLC chromatograph of 1.0 mg/mL SEQ ID NO: 1 at after 3 months stored at room temperature in saline at pH of 5.0. The retention time at 15.637 minutes correspond to SEQ ID NO: 1. FIG. 9 depicts the HPLC chromatograph of 1.0 mg/mL SEQ ID NO: 1 at after 3 months stored at −20° C. in saline at pH of 5.0. The retention time at 15.611 minutes correspond to SEQ ID NO: 1. FIG. 10 depicts the HPLC chromatograph of 1.0 mg/mL SEQ ID NO: 1 at after 3 months stored at 5° C. in PBS solution at pH of 7.4. The retention time at 15.585 minutes correspond to SEQ ID NO: 1. FIG. 11 depicts the HPLC chromatograph of 1.0 mg/mL SEQ ID NO: 1 at after 3 months stored at room temperature in PBS solution at pH of 7.4. The retention time at 15.593 minutes correspond to SEQ ID NO: 1. FIG. 12 depicts the HPLC chromatograph of 1.0 mg/mL SEQ ID NO: 1 at after 3 months stored at −20° C. in PBS solution at pH of 7.4. The retention time at 15.596 minutes correspond to SEQ ID NO: 1. FIG. 13 depicts the HPLC chromatograph of 1.0 mg/mL SEQ ID NO: 1 at after 3 months stored at 5° C. in tromethamine buffer at pH of 7.4. The retention time at 15.689 minutes correspond to SEQ ID NO: 1. FIG. 14 depicts the HPLC chromatograph of 1.0 mg/mL SEQ ID NO: 1 at after 3 months stored at room temperature in tromethamine buffer at pH of 7.4. The retention time at 15.529 minutes correspond to SEQ ID NO: 1. FIG. 15 depicts the HPLC chromatograph of 1.0 mg/mL SEQ ID NO: 1 at after 3 months stored at −20° C. in tromethamine buffer at pH of 7.4. The retention time at 15.555 minutes correspond to SEQ ID NO: 1. FIG. 16 depicts the HPLC chromatograph of 1.0 mg/mL SEQ ID NO: 1 at after 3 months stored at 5 ° C. in glycine buffer at pH of 7.4. The retention time at 15.564 minutes correspond to SEQ ID NO: 1. FIG. 17 depicts the HPLC chromatograph of 1.0 mg/mL SEQ ID NO: 1 at after 3 months stored at room temperature in glycine buffer at pH of 7.4. The retention time at 15.559 minutes correspond to SEQ ID NO: 1. FIG. 18 depicts the HPLC chromatograph of 1.0 mg/mL SEQ ID NO: 1 at after 3 months stored at −20° C. in glycine buffer at pH of 7.4. The retention time at 15.549 minutes correspond to SEQ ID NO: 1.

The lower pH peptide formulations showed higher stability, therefore a saline solution at pH 5.0 was better in comparison to the pH of 7.4 of any of the tested buffered formulations. Impurity growth observed at the tail of the SEQ ID NO: 1 peak starting at 1 month time point, may be temperature dependent. Ambient room temperature had higher values of impurities compared to 5° C. and −20° C. The saline based formulations had the least overall total impurities of about 1.4% at 3 month ambient temperature condition, due to the lower pH of the formulation. The impurities data at 3 month reveals that the total impurities recorded for SEQ ID NO: 1 formulated in Tromethamine at −20° C. and 5° C. was approximately half the number of observed in both PBS and Glycine formulations at the 3 month time point and match the impurity levels observed in the saline formulations. There was no significant difference in impurities values (approximately 3.5%) for PBS, Tromethamine, and Glycine based formulations at the 3 month ambient room temperature conditions. Storage of formulations at −20° C. for 3 months resulted in total impurities of 1.3%, 1.3%, 1.8% and 2.1% for 0.9% saline (pH 5.0), Tromethamine (pH 7.4), Glycine (pH 7.4) and phosphate buffered saline (pH 7.4), respectively. Storage at refrigerated condition (5° C.) resulted in total impurities of 1.3%, 1.3%, 2.4% and 2.7% for saline, Tromethamine, Glycine and Phosphate buffered saline formulations, respectively. The data indicates that the stability of these formulations improved in frozen storage as compared to ambient or refrigerated storage. Only, glycine formulation showed >95% assay at initial, 1, 2 and 3 months of storage at all the three storage temperatures.

The pH results during stability did not change and remained within target pH. The SEQ ID NO: 1 formulated in Tromethamine and Glycine resulted in drug product with low osmolality values (36 mOsmo/kg and 105 mOsm/kg) as compared to 290 mOsm/kg and 287 mOsm/kg for the saline and PBS based formulations respectively. This may have been due to the lack of osmotic agent (sodium chloride) for glycine and tromethamine formulations. All SEQ ID NO: 1 solutions were at all storage conditions met the acceptance criteria for visual appearance and particulate matter testing.

Stability was continued up to 6 months for saline based formulation (pH 5.0) due to least total impurities compared to SEQ ID NO: 1 formulated with tromethamine, PBS, and glycine. The total impurities recorded for 6M was 0.9%, 0.7%, and 0.8% at ambient, 5° C. and −20° C. respectively. Overall, there was no increase in purities growth as the 6 month is lower compared to 3 month at all storage conditions. Also, pH results were within target limits and did not change at the 6 month time point at all three conditions. The % Assay by US results at 6 months were 100.9%, 97.8%, and 93.8% at ambient, 5° C. and −20° C. respectively. The low assay results for the 5° C. sample may have been a result of lack of robustness in the method or error in sample preparation.

### Conclusion

High pH Buffers used in the SEQ ID NO: 1 formulations at the 1 mg/mL appeared to have an influence on observed impurities growth at the tail of the SEQ ID NO: 1 peak (main impurity). The total impurities were significantly less in SEQ ID NO: 1 formulated in the non-buffered 0.9% sodium chloride solution (saline solution), due to the low pH of this formulation. The impact of impurity growth was stronger at elevated (ambient room) temperature than the amount seen at low temperatures.

No significant difference was observed with impurities results by HPLC for SEQ ID NO: 1 formulation in 0.9% saline solution at all three different conditions (ambient, 5° C. and −20° C.) up to 6 months. Hence, 0.9% saline formulation is preferred, and the data suggest at least from impurity standpoint that SEQ ID NO: 1 is not susceptible to higher temperatures.

### Example 4: Stability of Peptide Formulations

This example illustrates the stability of peptide formulations tested after storage from 0 to 3 months at different temperatures.

The peptide formulation of SEQ ID NO: 1 dissolved in a saline solution with a pH of 5.0±0.1 was tested for stability under either −20° C. or 40° C. with 75% relative humidity (RH). The peptide formulations varied in concentrations (15.3 mg/mL, 36.7 mg/mL, and 72.4 mg/mL) and stored inverted in 2 mL amber glass vial containing 2 mL.

TABLE 14
Stability Summary of 15.3 mg/mL of SEQ ID NO: 1 Solution Stored at −20° C.

| | | | Timepoint | | | | | | | | | | | |
|---|---|---|---|---|---|---|---|---|---|---|---|---|---|---|
| | | | 0 Month | | | | 1 Month | | | | 2 Month | | | |
| | | | Project | | | | | | | | | | | |
| | | | 10275-005 | | | | 10275-008 | | | | 10275-009 | | | |
| | | | Pull Date | | | | | | | | | | | |
| | | | N/A | | | | 12 May 2021 | | | | 14 Jun. 2021 | | | |
| | | | Batch | | | | | | | | | | | |
| | | | N/A | | | | TB362821 | | | | TB365254 | | | |
| Test | Method | Acceptance Criteria | Results | | | | Results | | | | Results | | | |
| Visual | 25481 | Report results | Clear, colorless liquid. Free of visible particulates. | | | | Clear, colorless liquid. Free of visible particulates. | | | | Clear, colorless liquid. Free of visible | | | |
| Appearance | | | Packaged in a brown glass vial with a legible | | | | Packaged in a brown glass vial with a legible | | | | particulates. Packaged in a brown glass | | | |
| | | | label, metal crimp seal, and grey rubber stopper. | | | | label, metal crimp seal, and grey rubber stopper. | | | | vial with a legible label, metal crimp | | | |
| | | | | | | | | | | | seal, and grey rubber stopper. | | | |
| | | Tested | 13 Apr. 2021 | | | | 14 May 2021 | | | | 17 Jun. 2021 | | | |
| pH | 05627 | Report Results | Average Reading: 4.9 | | | | Average Reading: 5.0 | | | | Average Reading: 4.9 | | | |
| | | Tested | 13 Apr. 2021 | | | | 14 May 2021 | | | | 17 Jun. 2021 | | | |
| HPLC-UV | 78917 | Assay: 90.0-110.0% | Conc. | % | % Total | % | Conc. | % | % Total | % | % | Impurities | % Total | % |
| Assay/RS | | Related Substances: Report Results | (mg/mL) | Assay | Impurities | Purity | (mg/mL) | Assay | Impurities | Purity | Assay | RRT/% | Impurities | Purity |
| | | | 15.3 | 99.8 | 1.0 | 99.0 | 15.3 | 103.4 | 0.9 | 99 | 106.0 | 1.02/0.9 | 0.9 | 99 |
| | | | | % area | % area | Mean | | % area | % area | Mean | | | | |
| | | | ID | (Inj-1) | (Inj-2) | % Area | ID | (Inj-1) | (Inj-2) | % Area | | | | |
| | | | SEQ ID | 98.95 | 98.97 | 99.0 | SEQ ID | 99.12 | 99.13 | 99.1 | | | | |
| | | | NO: 1 | | | | NO: 1 | | | | | | | |
| | | | RRT 1.02 | 1.05 | 1.03 | 1.0 | RRT 1.02 | 0.88 | 0.87 | 0.9 | | | | |
| | | Tested | 15 Apr. 2021 | | | | 17 May 2021 | | | | 22 Jun. 2021 | | | |
| UV-Vis | 78933 | Assay: 90.0-110.0% | 102.0 | | | | 101.5 | | | | 104.0 | | | |
| Assay | | Tested | 15 Apr. 2021 | | | | 14 May 2021 | | | | 25 Jun. 2021 | | | |
| Osmolality | 69153 | Report Result (mOsmol/kg) | Average Reading: 82 | | | | Average Reading: 82 | | | | Average Reading: 82 | | | |
| | | Tested | 13 Apr. 2021 | | | | 17 May 2021 | | | | 17 Jun. 2021 | | | |
| | | | Timepoint | | | | | | | | | | | |
| | | | 3 Month | | | | 6 Month | | | | 9 Month | | | |
| | | | Project | | | | | | | | | | | |
| | | | 10275-010 | | | | 10275-011 | | | | 10275-012 | | | |
| | | | Pull Date | | | | | | | | | | | |
| | | | 12 Jul. 2021 | | | | 12 Oct. 2021 | | | | 12 Jan. 2022 | | | |
| | | | Batch | | | | | | | | | | | |
| | | | TB367241 | | | | TB376264 | | | | TB382911 | | | |
| Test | Method | Acceptance Criteria | Results | | | | Results | | | | Results | | | |
| Visual | 25481 | Report results | Clear, colorless liquid. Free of visible particulates. | | | | Clear, colorless liquid. Free of visible particulates, | | | | Not Tested | | | |
| Appearance | | | Packaged in a brown glass vial with a legible | | | | packaged in a brown glass vial with a legible | | | | | | | |
| | | | label, metal crimp seal, and grey rubber stopper. | | | | label, metal crimp seal, and grey rubber stopper. | | | | | | | |
| | | Tested | 15 Jul. 2021 | | | | 22 Oct. 2021 | | | | | | | |
| pH | 05627 | Report Results | Average Reading: 5.0 | | | | 5.0 | | | | Not Tested | | | |
| | | Tested | 15 Jul. 2021 | | | | 22 Oct. 2021 | | | | | | | |
| HPLC-UV | 78917 | Assay: 90.0-110.0% | % | Impurities | % Total | % | % | Impurities | % Total | % | % | Impurities | % Total | % |
| Assay/RS | | Related Substances: Report Results | Assay | RRT/% | Impurities | Purity | Assay | RRT/% | Impurities | Purity | Assay | RRT/% | Impurities | Purity |
| | | | 99.7 | 1.02/0.9 | 0.9 | 99 | 102.1 | 1.02/0.9 | 0.9 | 99 | 101.0 | 1.01/0.3 | 0.3 | 100 |

| | | | | | |
|---|---|---|---|---|---|
| | | Tested | 15 Jul. 2021 | 13 Oct. 2021 | 30 Jan. 2022 |
| UV-Vis | 78933 | Assay: 90.0-110.0% | 105.1 | 103.9 | 103.1 |
| Assay | | Tested | 22 Jul. 2021 | 26 Oct. 2021 | 19 Jan. 2022 |
| Osmolality | 69153 | Report Result (mOsmol/kg) | Average Reading: 82 | 84 | 83 |
| | | Tested | 15 Jul. 2021 | 28 Oct. 2021 | 17 Jan. 2022 |

TABLE 15
Stability Summary of 15.3 mg/mL of SEQ ID NO: 1 Solution Stored at 40° C. and 75% RH

| | | | Timepoint | | | | | | | | | | | |
|---|---|---|---|---|---|---|---|---|---|---|---|---|---|---|
| | | | 1 Mouth | | | | 2 Month | | | | 3 Month | | | |
| | | | Project | | | | | | | | | | | |
| | | | 10275-008 | | | | 10275-009 | | | | 10275-010 | | | |
| | | | Pull Date | | | | | | | | | | | |
| | | | 12 May 2021 | | | | 14 Jun. 2021 | | | | 12 Jul. 2021 | | | |
| | | | Batch | | | | | | | | | | | |
| | | | TB362822 | | | | TB365255 | | | | TB367242 | | | |
| Test | Method | Acceptance Criteria | Results | | | | Results | | | | Results | | | |
| Visual | 25481 | Report results | Clear, colorless liquid. Free of visible particulates. | | | | Clear, colorless liquid. Free of visible particulates. | | | | Clear, colorless liquid. Free of visible particulates. | | | |
| Appearance | | Tested | Packaged in a brown glass vial with a legible | | | | Packaged in a brown glass vial with a legible | | | | Packaged in a brown glass vial with a legible | | | |
| | | | label, metal crimp seal, and grey rubber stopper. | | | | label, metal crimp seal, and grey rubber stopper. | | | | label, metal crimp seal, and grey rubber stopper. | | | |
| pH | 05627 | Report Results | Average Reading: 4.9 | | | | Average Reading: 5.0 | | | | Average Reading: 5.0 | | | |
| | | Tested | 14 May 2021 | | | | 17 Jun. 2021 | | | | 15 Jul. 2021 | | | |
| HPLC-UV | 78917 | Assay: 90.0-110.0% | Conc. | % | % Total | % | % | Impurities | % Total | % | % | Impurities | % Total | % |
| Assay/RS | | Related Substances: Report Results | (mg/mL) | Assay | Impurities | Purity | Assay | RRT/% | Impurities | Purity | Assay | RRT/% | Impurities | Purity |
| | | | 15.3 | 96.0 | 1.0 | 99 | 103.8 | 1.02/1.0 | 1.4 | 99 | 96.8 | 1.02/1.1 | 1.3 | 99 |
| | | | | | | | | 1.08/0.2 | | | | 1.25/0.2 | | |
| | | | | | | | | 1.10/0.2 | | | | | | |
| | | | | % area | % area | Mean | | | | | | | | |
| | | | ID | (Inj-1) | (Inj-2) | % Area | | | | | | | | |
| | | | SEQ ID | 99.03 | 99.05 | 99.0 | | | | | | | | |
| | | | NO: 1 | | | | | | | | | | | |
| | | | RRT 1.02 | 0.97 | 0.95 | 1.0 | | | | | | | | |

| | | Tested | 17 May 2021 | | | | 22 Jun. 2021 | 15 Jul. 2021 |
|---|---|---|---|---|---|---|---|---|
| UV-Vis | 78933 | Assay: 90.0-110.0% | 102.0 | | | | 102.2 | 103.9 |
| Assay | | Tested | 14 May 2021 | | | | 25 Jun. 2021 | 22 Jul. 2021 |
| Osmolality | 69153 | Report Result (mOsmol/kg) | Average Reading: 82 | | | | Average Reading: 83 | Average Reading: 80 |
| | | Tested | 17 May 2021 | | | | 17 Jun. 2021 | 15 Jul. 2021 |
| | | | Timepoint | | | | | |
| | | | 6 Month | | | | | |
| | | | Project | | | | | |
| | | | 10275-011 | | | | | |
| | | | Pull Date | | | | | |
| | | | 12 Oct. 2021 | | | | | |
| | | | Batch | | | | | |
| | | | TB376265 | | | | | |
| Test | Method | Acceptance Criteria | Results | | | | | |
| Visual | 25481 | Report results | Clear, colorless liquid. Free of visible particulates, | | | | | |
| Appearance | | | packaged in a brown glass vial with a legible | | | | | |
| | | | label, metal crimp seal, and grey rubber stopper. | | | | | |
| | | Tested | 22 Oct. 2021 | | | | | |
| pH | 05627 | Report Results | 5.0 | | | | | |
| | | Tested | 22 Oct. 2021 | | | | | |
| HPLC-UV | 78917 | Assay: 90.0-110.0% | Conc. | % | % Total | % | | |
| Assay/RS | | Related Substances: Report Results | (mg/mL) | Assay | Impurities | Purity | | |
| | | | 97.5 | 0.94/0.2 | 2.5 | 98 | | |
| | | | | 0.95/0.2 | | | | |
| | | | | 0.97/0.3 | | | | |
| | | | | 1.02/1.4 | | | | |
| | | | | 1.10/0.3 | | | | |
| | | | | 1.32/0.2 | | | | |

| | | | |
|---|---|---|---|
| | | Tested | 13 Oct. 2021 |
| UV-Vis | 78933 | Assay: 90.0-110.0% | 102.5 |
| Assay | | Tested | 26 Oct. 2021 |
| Osmolality | 69153 | Report Result (mOsmol/kg) | 85 |
| | | Tested | 28 Oct. 2021 |

TABLE 16
Stability Summary of 36.7 mg/mL of SEQ ID NO: 1 Solution Stored at −20° C.

| | | | Timepoint | | | |
|---|---|---|---|---|---|---|
| | | | 0 Month | | | |
| | | | Project | | | |
| | | | 10275-005 | | | |
| | | | Pull Date | | | |
| | | | N/A | | | |
| | | | Batch | | | |
| | | | N/A | | | |
| Test | Method | Acceptance Criteria | Results | | | |
| Visual | 25481 | Report results | Clear, colorless liquid. Free | | | |
| Appearance | | | of visible particulates. Packaged in | | | |
| | | | a brown glass vial with a legible | | | |
| | | | label, metal crimp seal, and grey | | | |
| | | | rubber stopper. | | | |
| | | Tested | 13 Apr. 2021 | | | |
| pH | 05627 | Report Results | Average Reading: 4.9 | | | |
| | | Tested | 13 Apr. 2021 | | | |
| HPLC-UV | 78917 | Assay: 90.0-110.0% | Conc. | % | % Total | % |
| Assay/RS | | Related Substances: Report Results | (mg/mL) | Assay | Impurities | Purity |
| | | | 36.7 | 97.9 | 1.0 | 99.0 |
| | | | | % area | % area | Mean |
| | | | ID | (Inj-1) | (Inj-2) | % Area |
| | | | SEQ ID NO: 1 | 98.98 | 98.97 | 99.0 |
| | | | RRT 1.02 | 1.02 | 1.03 | 1.0 |

| | | | |
|---|---|---|---|
| | | Tested | 15 Apr. 2021 |
| UV-Vis | 78933 | Assay: 90.0-110.0% | 98.1 |
| Assay | | Tested | 15 Apr. 2021 |
| Osmolality | 69153 | Report Result (mOsmol/kg) | Average Reading: 190 |
| | | Tested | 13 Apr. 2021 |

| | Timepoint | |
|---|---|---|
| | 1 Month | 2 Month |
| | Project | |
| | 10275-008 | 10275-009 |
| | Pull Date | |
| | 12 May 2021 | 14 Jun. 2021 |
| | Batch | |
| | TB362823 | TB365257 |
| Test | Results | Results |
| Visual | Clear, colorless liquid. Free | Clear, colorless liquid. Free |
| Appearance | of visible particulates. Packaged in | of visible particulates. Packaged in |
| | a brown glass vial with a legible | a brown glass vial with a legible |
| | label, metal crimp seal, and gray | label, metal crimp seal, and gray |
| | rubber stopper. | rubber stopper. |
| | 14 May 2021 | 17 Jun. 2021 |
| pH | Average Reading: 4.8 | Average Reading: 4.9 |
| | 14 May 2021 | 17 Jun. 2021 |

| | | | | | | | | |
|---|---|---|---|---|---|---|---|---|
| HPLC-UV | Conc. | % | % Total | % | % | Impurities | % Total | % |
| Assay/RS | (mg/mL) | Assay | Impurities | Purity | Assay | RRT/% | Impurities | Purity |
| | 36.7 | 98.6 | 0.9 | 99 | 103.2 | 1.02/0.9 | 0.9 | 99 |
| | | % area | % area | Mean | | | | |
| | ID | (Inj-1) | (Inj-2) | % Area | | | | |
| | SEQ ID NO: 1 | 99.13 | 99.12 | 99.1 | | | | |
| | RRT 1.02 | 0.87 | 0.88 | 0.9 | | | | |

| | | |
|---|---|---|
| | 17 May 2021 | 22 Jun. 2021 |
| UV-Vis | 100.0 | 100.6 |
| Assay | 14 May 2021 | 25 Jun. 2021 |
| Osmolality | Average Reading: 188 | Average Reading: 190 |
| | 17 May 2021 | 17 Jun. 2021 |

| | | | Timepoint | | | |
|---|---|---|---|---|---|---|
| | | | 3 Month | | | |
| | | | Project | | | |
| | | | 10275-010 | | | |
| | | | Pull Date | | | |
| | | | 12 Jul. 2021 | | | |
| | | | Batch | | | |
| | | | TB367246 | | | |
| Test | Method | Acceptance Criteria | Results | | | |
| Visual | 25481 | Report results | Clear, colorless liquid. Free | | | |
| Appearance | | | of visible particulates. Packaged in | | | |
| | | | a brown glass vial with a legible | | | |
| | | | label, metal crimp seal, and grey | | | |
| | | | rubber stopper. | | | |
| | | Tested | 15 Jul. 2021 | | | |
| pH | 05627 | Report Results | Average Reading: 5.0 | | | |
| | | Tested | 15 Jul. 2021 | | | |
| HPLC-UV | 78917 | Assay: 90.0-110.0% | % | Impurities | % Total | % |
| Assay/RS | | Related Substances: Report Results | Assay | RRT/% | Impurities | Purity |
| | | | 101.7 | 1.02/0.9 | 0.9 | 99 |

| | | | |
|---|---|---|---|
| | | Tested | 15 Jul. 2021 |
| UV-Vis | 78933 | Assay: 90.0-110.0% | 101.7 |
| Assay | | Tested | 22 Jul. 2021 |
| Osmolality | 69153 | Report Result (mOsmol/kg) | Average Reading: 193 |
| | | Tested | 15 Jul. 2021 |

| | Timepoint | | | | | | | |
|---|---|---|---|---|---|---|---|---|
| | 6 Month | | | | 9 Month | | | |
| | Project | | | | | | | |
| | 10275-011 | | | | 10275-012 | | | |
| | Pull Date | | | | | | | |
| | 12 Oct. 2021 | | | | 12 Jan. 2022 | | | |
| | Batch | | | | | | | |
| | TB376266 | | | | TB382912 | | | |
| Test | Results | | | | Results | | | |
| Visual | Clear, colorless liquid, free | | | | Not Tested | | | |
| Appearance | of visible particulates, packaged in | | | | | | | |
| | a brown glass vial with a legible | | | | | | | |
| | label, metal crimp seal, and grey | | | | | | | |
| | rubber stopper | | | | | | | |
| | 22 Oct. 2021 | | | | | | | |
| pH | 4.9 | | | | Not Tested | | | |
| | 22 Oct. 2021 | | | | | | | |
| HPLC-UV | % | Impurities | % Total | % | % | Impurities | % Total | % |
| Assay/RS | Assay | RRT/% | Impurities | Purity | Assay | RRT/% | Impurities | Purity |
| | 99.8 | 1.02/0.9 | 0.9 | 99 | 96.9 | 1.01/0.3 | 0.3 | 100 |

| | | |
|---|---|---|
| | 13 Oct. 2021 | 30 Jan. 2022 |
| UV-Vis | 99.5 | 100.3 |
| Assay | 26 Oct. 2021 | 19 Jan. 2022 |
| Osmolality | 190 | 190 |
| | 28 Oct. 2021 | 17 Jan. 2022 |

TABLE 17
Stability Summary of 36.7 mg/mL of SEQ ID NO: 1 Solution Stored at 40° C. and 75% RH

| | | | Timepoint | | | |
|---|---|---|---|---|---|---|
| | | | 1 Month | | | |
| | | | Project | | | |
| | | | 10275-008 | | | |
| | | | Pull Date | | | |
| | | | 12 May 2021 | | | |
| | | | Batch | | | |
| | | | TB362824 | | | |
| Test | Method | Acceptance Criteria | Results | | | |
| Visual | 25481 | Report results | Clear, colorless liquid. Free | | | |
| Appearance | | | of visible particulates. Packaged in | | | |
| | | | a brown glass vial with a legible | | | |
| | | | label, metal crimp seal, and gray | | | |
| | | | rubber stopper. | | | |
| | | Tested | 14 May 2021 | | | |
| pH | 05627 | Report Results | Average Reading: 4.8 | | | |
| | | Tested | 14 May 2021 | | | |
| HPLC-UV | 78917 | Assay: 90.0-110.0% | Conc. | % | % Total | % |
| Assay/RS | | Related Substances: Report Results | (mg/mL) | Assay | Impurities | Purity |
| | | | 36.7 | 97.5 | 0.9 | 99 |
| | | | | % area | % area | Mean |
| | | | ID | (Inj-1) | (Inj-2) | % Area |
| | | | SEQ ID NO: 1 | 99.06 | 99.09 | 99.1 |
| | | | RRT 1.02 | 0.94 | 0.91 | 0.9 |

| | | | |
|---|---|---|---|
| | | Tested | I7 May 2021 |
| UV-Vis | 78933 | Assay: 90.0-110.0% | 100.3 |
| Assay | | Tested | I4 May 2021 |
| Osmolality | 69153 | Report Result (mOsmol/kg) | Average Reading: 188 |
| | | Tested | I7 May 2021 |

| | Timepoint | | | | | | | |
|---|---|---|---|---|---|---|---|---|
| | 2 Month | | | | 3 Month | | | |
| | Project | | | | | | | |
| | 10275-009 | | | | 10275-010 | | | |
| | Pull Date | | | | | | | |
| | 14 Jun. 2021 | | | | 12 Jul. 2021 | | | |
| | Batch | | | | | | | |
| | TB365258 | | | | TB367247 | | | |
| Test | Results | | | | Results | | | |
| Visual | Clear, colorless liquid. Free | | | | Clear, colorless liquid. Free | | | |
| Appearance | of visible particulates. Packaged in | | | | of visible particulates. Packaged in | | | |
| | a brown glass vial with a legible | | | | a brown glass vial with a legible | | | |
| | label, metal crimp seal, and gray | | | | label, metal crimp seal, and grey | | | |
| | rubber stopper. | | | | rubber stopper. | | | |
| | 17 Jun. 2021 | | | | 15 Jul. 2021 | | | |
| pH | Average Reading: 4.9 | | | | Average Reading: 5.0 | | | |
| | 17 Jun. 2021 | | | | 15 Jul. 2021 | | | |
| HPLC-UV | % | Impurities | % Total | % | % | Impurities | % Total | % |
| Assay/RS | Assay | RRT/% | Impurities | Purity | Assay | RRT/% | Impurities | Purity |
| | 101.7 | 1.02/1.0 | 1.2 | 99 | 97.3 | 0.98/0.2 | 1.5 | 99 |
| | | 1.32/0.2 | | | | 1.02/1.0 | | |
| | | | | | | 1.20/0.3 | | |

| | | |
|---|---|---|
| | 22 Jun. 2021 | 15 Jul. 2021 |
| UV-Vis | 101.3 | 99.2 |
| Assay | 25 Jun. 2021 | 22 Jul. 2021 |
| Osmolality | Average Reading: 189 | Average Reading: 191 |
| | I7 Jun. 2021 | 15 Jul. 2021 |

| | | | Timepoint | | | |
|---|---|---|---|---|---|---|
| | | | 6 Month | | | |
| | | | Project | | | |
| | | | 10275-011 | | | |
| | | | Pull Date | | | |
| | | | 12 Oct. 2021 | | | |
| | | | Batch | | | |
| | | | TB376267 | | | |
| Test | Method | Acceptance Criteria | Results | | | |
| Visual | 25481 | Report results | Clear, colorless liquid, free | | | |
| Appearance | | | of visible particulates, packaged in | | | |
| | | | a brown glass vial with a legible | | | |
| | | | label, metal crimp seal, and grey | | | |
| | | | rubber stopper | | | |
| | | Tested | 22 Oct. 2021 | | | |
| pH | 05627 | Report Results | 4.8 | | | |
| | | Tested | 22 Oct. 2021 | | | |
| HPLC-UV | 78917 | Assay: 90.0-110.0% | % | Impurities | % Total | % |
| Assay/RS | | Related Substances: Report Results | Assay | RRT/% | Impurities | Purity |
| | | | 94.8 | 0.64/0.2 | 3.0 | 97 |
| | | | | 0.88/0.2 | | |
| | | | | 0.95/0.2 | | |
| | | | | 0.97/0.4 | | |
| | | | | 1.02/1.4 | | |
| | | | | 1.10/0.2 | | |
| | | | | 1.32/0.3 | | |

| | | | |
|---|---|---|---|
| | | Tested | 13 Oct. 2021 |
| UV-Vis | 78933 | Assay: 90.0-110.0% | 100.1 |
| Assay | | Tested | 26 Oct. 2021 |
| Osmolality | 69153 | Report Result (mOsmol/kg) | 193 |
| | | Tested | 28 Oct. 2021 |

TABLE 18
Stability Summary of 72.4 mg/mL of SEQ ID NO: 1 Solution Stored at −20° C.

| | | | Timepoint | | | |
|---|---|---|---|---|---|---|
| | | | 0 Month | | | |
| | | | Project | | | |
| | | | 10275-005 | | | |
| | | | Pull Date | | | |
| | | | N/A | | | |
| | | | Batch | | | |
| | | | N/A | | | |
| Test | Method | Acceptance Criteria | Results | | | |
| Visual | 25481 | Report results | Clear, colorless liquid. Free | | | |
| Appearance | | | of visible particulates. Packaged in | | | |
| | | | a brown glass vial with a legible | | | |
| | | | label, metal crimp seal, and grey | | | |
| | | | rubber stopper. | | | |
| | | Tested | 13 Apr. 2021 | | | |
| pH | 05627 | Report Results | Average Reading: 4.9 | | | |
| | | Tested | 13 Apr. 2021 | | | |
| HPLC-UV | 78917 | Assay: 90.0-110.0% | Conc. | % | % Total | % |
| Assay/RS | | Related Substances: Report Results | (mg/mL) | Assay | Impurities | Puritvy |
| | | | 72.4 | 94.2 | 1.0 | 99.1 |
| | | | | % area | % area | Mean |
| | | | ID | (Inj-1) | (Inj-2) | % Area |
| | | | SEQ ID NO: 1 | 99.03 | 99.07 | 99.1 |
| | | | RRT 1.02 | 0.97 | 0.93 | 1.0 |

| | | | |
|---|---|---|---|
| | | Tested | 15 Apr. 2021 |
| UV-Vis | 78933 | Assay: 90.0-110.0% | 90.1 |
| Assay | | Tested | 15 Apr. 2021 |
| Osmolality | 69153 | Report Result (mOsmol/kg) | Average Reading: 371 |
| | | Tested | 13 Apr. 2021 |

| | Timepoint | | | | | | | |
|---|---|---|---|---|---|---|---|---|
| | 1 Month | | | | 2 Month | | | |
| | Project | | | | | | | |
| | 10275-008 | | | | 10275-009 | | | |
| | Pull Date | | | | | | | |
| | 12 May 2021 | | | | 14 Jun. 2021 | | | |
| | Batch | | | | | | | |
| | TB362825 | | | | TB365259 | | | |
| Test | Results | | | | Results | | | |
| Visual | Clear, colorless liquid. Free | | | | Clear, colorless liquid. Free | | | |
| Appearance | of visible particulates. Packaged in | | | | of visible particulates. Packaged in | | | |
| | a brown glass vial with a legible | | | | a brown glass vial with a legible | | | |
| | label, metal crimp seal, and gray | | | | label, metal crimp seal, and gray | | | |
| | rubber stopper. | | | | rubber stopper. | | | |
| | 14 May 2021 | | | | 17 Jun. 2021 | | | |
| pH | Average Reading: 4.7 | | | | Average Reading: 4.8 | | | |
| | 14 May 2021 | | | | 17 Jun. 2021 | | | |
| HPLC-UV | Conc. | % | Total % | % | % | Impurities | % Total | % |
| Assay/RS | (mg/mL) | Assay | Impurities | Puritvy | Assay | RRT/% | Impurities | Purity |
| | 72.4 | 97.7 | 0.9 | 99 | 101.1 | 1.02/0.9 | 0.9 | 99 |
| | | % area | % area | Mean | | | | |
| | ID | (Inj-1) | (Inj-2) | % Area | | | | |
| | SEQ ID NO: 1 | 99.12 | 99.13 | 99.1 | | | | |
| | RRT 1.02 | 0.88 | 0.87 | 0.9 | | | | |

| | | |
|---|---|---|
| | 17 May 2021 | 22 Jun. 2021 |
| UV-Vis | 97.6 | 98.2 |
| Assay | 14 May 2021 | 25 Jun. 2021 |
| Osmolality | Average Reading: 374 | Average Reading: 376 |
| | 17 May 2021 | 17 Jun. 2021 |

| | | | Timepoint | | | |
|---|---|---|---|---|---|---|
| | | | 3 Month | | | |
| | | | Project | | | |
| | | | 10275-010 | | | |
| | | | Pull Date | | | |
| | | | 12 Jul. 2021 | | | |
| | | | Batch | | | |
| | | | TB367248 | | | |
| Test | Method | Acceptance Criteria | Results | | | |
| Visual | 25481 | Report results | Clear, colorless liquid. Free | | | |
| Appearance | | | of visible particulates. Packaged in | | | |
| | | | a brown glass vial with a legible | | | |
| | | | label, metal crimp seal, and grey | | | |
| | | | rubber stopper. | | | |
| | | Tested | 15 Jul. 2021 | | | |
| pH | 05627 | Report Results | Average Reading: 5.0 | | | |
| | | Tested | 15 Jul. 2021 | | | |
| HPLC-UV | 78917 | Assay: 90.0-110.0% | % | Impurities | % Total | % |
| Assay/RS | | Related Substances: Report Results | Assay | RRT/% | Impurities | Purity |
| | | | 97.4 | 1.02/0.8 | 1.0 | 99 |
| | | | | 1.21/0.2 | | |

| | | | |
|---|---|---|---|
| | | Tested | 15 Jul. 2021 |
| UV-Vis | 78933 | Assay: 90.0-110.0% | 94.5 |
| Assay | | Tested | 22 Jul. 2021 |
| Osmolality | 69153 | Report Result (mOsmol/kg) | Average Reading: 374 |
| | | Tested | 15 Jul. 2021 |

| | Timepoint | | | | | | | |
|---|---|---|---|---|---|---|---|---|
| | 6 Month | | | | 9 Month | | | |
| | Project | | | | | | | |
| | 10275-011 | | | | 10275-012 | | | |
| | Pull Date | | | | | | | |
| | 12 Oct. 2021 | | | | 12 Jan. 2022 | | | |
| | Batch | | | | | | | |
| | TB376268 | | | | TB382913 | | | |
| Test | Results | | | | Results | | | |
| Visual | Clear, colorless liquid, free | | | | Not Tested | | | |
| Appearance | of visible particulates, packaged in | | | | | | | |
| | a brown glass vial with a legible | | | | | | | |
| | label, metal crimp seal, and grey | | | | | | | |
| | rubber stopper | | | | | | | |
| | 22 Oct. 2021 | | | | | | | |
| pH | 4.8 | | | | Not Tested | | | |
| | 22 Oct. 2021 | | | | | | | |
| HPLC-UV | % | Impurities | % Total | % | % | Impurities | % Total | % |
| Assay/RS | Assay | RRT/% | Impurities | Purity | Assay | RRT/% | Impurities | Purity |
| | 95.1 | 1.02/0.9 | 0.9 | 99 | 95.9 | 1.01/0.3 | 0.5 | 100 |
| | | | | | | 1.06/0.2 | | |

| | | |
|---|---|---|
| | 13 Oct. 2021 | 30 Jan. 2022 |
| UV-Vis | 97.0 | 99.0 |
| Assay | 26 Oct. 2021 | 19 Jan. 2022 |
| Osmolality | 195 | 375 |
| | 28 Oct. 2021 | 17 Jan. 2022 |

TABLE 19
Stability Summary of 72.4 mg/mL of SEQ ID NO: 1 Solution Stored at 40° C. and 75% RH

| | | | Timepoint | | | |
|---|---|---|---|---|---|---|
| | | | 1 Month | | | |
| | | | Project | | | |
| | | | 10275-008 | | | |
| | | | Pull Date | | | |
| | | | 12 May 2021 | | | |
| | | | Batch | | | |
| | | | TB362826 | | | |
| Test | Method | Acceptance Criteria | Results | | | |
| Visual | 25481 | Report results | Clear, colorless liquid. Free | | | |
| Appearance | | | of visible particulates. Packaged in | | | |
| | | | a brown glass vial with a legible | | | |
| | | | label, metal crimp seal, and gray | | | |
| | | | rubber stopper. | | | |
| | | Tested | 14 May 2021 | | | |
| pH | 05627 | Report Results | Average Reading: 4.7 | | | |
| | | Tested | 14 May 2021 | | | |
| HPLC-UV | 78917 | Assay: 90.0-110.0% | Conc. | % | % Total | % |
| Assay/RS | | Related Substances: Report Results | (mg/mL) | Assay | Impurities | Purity |
| | | | 72.4 | 95.9 | 1.1 | 99 |
| | | | | % area | % area | Mean |
| | | | ID | (Inj-1) | (Inj-2) | % Area |
| | | | SEQ ID NO: 1 | 98.91 | 98.92 | 98.9 |
| | | | RRT 1.02 | 0.92 | 0.90 | 0.9 |
| | | | RRT 1.32 | 0.17 | 0.18 | 0.2 |

| | | | |
|---|---|---|---|
| | | Tested | I7 May 2021 |
| UV-Vis | 78933 | Assay: 90.0-110.0% | 97.8 |
| Assay | | Tested | I4 May 2021 |
| Osmolality | 69153 | Report Result (mOsmol/kg) | Average Reading: 374 |
| | | Tested | I7 May 2021 |

| | Timepoint | | | | | | | |
|---|---|---|---|---|---|---|---|---|
| | 2 Month | | | | 3 Month | | | |
| | Project | | | | | | | |
| | 10275-009 | | | | 10275-010 | | | |
| | Pull Date | | | | | | | |
| | 14 Jun. 2021 | | | | 12 Jul. 2021 | | | |
| | Batch | | | | | | | |
| | TB365262 | | | | TB367249 | | | |
| Test | Results | | | | Results | | | |
| Visual | Clear, colorless liquid. Free | | | | Clear, colorless liquid. Free | | | |
| Appearance | of visible particulates. Packaged in | | | | of visible particulates. Packaged in | | | |
| | a brown glass vial with a legible | | | | a brown glass vial with a legible | | | |
| | label, metal crimp seal, and gray | | | | label, metal crimp seal, and grey | | | |
| | rubber stopper. | | | | rubber stopper. | | | |
| | 17 Jun. 2021 | | | | 15 Jul. 2021 | | | |
| pH | Average Reading: 4.9 | | | | Average Reading: 5.0 | | | |
| | 17 Jun. 2021 | | | | 15 Jul. 2021 | | | |
| HPLC-UV | % | Impurities | % Total | % | % | Impurities | % Total | % |
| Assay/RS | Assay | RRT/% | Impurities | Purity | Assay | RRT/% | Impurities | Purity |
| | 98.4 | 0.98/0.2 | 1.5 | 99 | 90.5 | 0.96/0.2 | 1.8 | 98 |
| | | 1.02/1.0 | | | | 0.98/0.2 | | |
| | | 1.32/0.3 | | | | 1.02/1.0 | | |
| | | | | | | 1.21/0.4 | | |

| | | |
|---|---|---|
| | 22 Jun. 2021 | 15 Jul. 2021 |
| UV-Vis | 98.6 | 93.4 |
| Assay | 25 Jun. 2021 | 22 Jul. 2021 |
| Osmolality | Average Reading: 379 | Average Reading: 371 |
| | I7 Jun. 2021 | 15 Jul. 2021 |

| | | | Timepoint | | | |
|---|---|---|---|---|---|---|
| | | | 6 Month | | | |
| | | | Project | | | |
| | | | 10275-011 | | | |
| | | | Pull Date | | | |
| | | | 12 Oct. 2021 | | | |
| | | | Batch | | | |
| | | | TB376269 | | | |
| Test | Method | Acceptance Criteria | Results | | | |
| Visual | 25481 | Report results | Clear, slightly yellow liquid, free | | | |
| Appearance | | | of visible particulates, packaged in | | | |
| | | | a brown glass vial with a legible | | | |
| | | | label, metal crimp seal, and grey | | | |
| | | | rubber stopper | | | |
| | | Tested | 22 Oct. 2021 | | | |
| pH | 05627 | Report Results | 4.8 | | | |
| | | Tested | 22 Oct. 2021 | | | |
| HPLC-UV | 78917 | Assay: 90.0-110.0% | % | Impurities | % Total | % |
| Assay/RS | | Related Substances: Report Results | Assay | RRT/% | Impurities | Purity |
| | | | 87.0* | 0.70/0.2 | 3.6 | 96 |
| | | | | 0.83/0.2 | | |
| | | | | 0.88/0.2 | | |
| | | | | 0.95/0.4 | | |
| | | | | 0.97/0.5 | | |
| | | | | 1.02/1.4 | | |
| | | | | 1.32/0.7 | | |

| | | | |
|---|---|---|---|
| | | Tested | 13 Oct. 2021 |
| UV-Vis | 78933 | Assay: 90.0-110.0% | 95.4 |
| Assay | | Tested | 26 Oct. 2021 |
| Osmolality | 69153 | Report Result (mOsmol/kg) | 194 |
| | | Tested | 28 Oct. 2021 |

TABLE 20
Stability Summary of 81.4 mg/mL of SEQ ID NO: 1 Solution Stored at −20° C.

| | | | Timepoint | | |
|---|---|---|---|---|---|
| | | | 0 Month | 1 Month | 2 Month |
| | | | Project | | |
| | | | 10275-018 | 10275-021 | 10275-022 |
| | | | Pull Date | | |
| | | | N/A | 5 Nov. 2021 | 6 Dec. 2021 |
| | | | Batch | | |
| | | | N/A | TB378317 | TB380496 |
| Test | Method | Acceptance Criteria | Results | Results | Results |
| Visual | 25481 | Report results | Clear colorless liquid free of | Clear, colorless liquid | Amber glass vial with aluminum |
| Appearance | | | visible particles inside brown glass | | seal, legible label, and blue flip off |
| | | | vial with legible label, metal | | cap over a gray stopper containing |
| | | | crimp seal, and grey rubber stopper | | a clear colorless liquid, visibly |
| | | | | | free of particulate matter. |
| | | Tested | 4 Oct. 2021 | 17 Nov. 2021 | 3 Jan. 2022 |
| pH | 05627 | Report Results | 4.6 | 4.8 | 4.9 |
| | | Tested | 4 Oct. 2021 | 19 Nov. 2021 | 3 Jan. 2022 |
| HPLC-UV | 78917 | 90.0-110.0% (% LC) | 108.6 | 103.8 | 107.8 |
| Assay | | Tested | 4 Oct. 2021 | 1 Dec. 2021 | 14 Dec. 2021 |
| Related | 78917 | Individual Impurity: | 1.02/0.9 | 1.02/1.0 | 1.02/0.9 |
| Substances | | Report Results (RRT/%) | | | |
| | | Total Impurities: | 0.9 | 1.0 | 0.9 |
| | | Report Results (%) | | | |
| | | Purity: Report Results (%) | 99 | 99 | 99 |
| | | Tested | 4 Oct. 2021 | 1 Dec. 2021 | 14 Dec. 2021 |
| UV-Vis | 78933 | 90.0-110.0% (% LC) | 105.0 | Not Scheduled | Not Scheduled |
| Assay | | Tested | 28 Oct. 2021 | | |
| Osmolality | 69153 | Report Result (mOsm/kg) | 485 | 489 | 488 |
| | | Tested | 4 Oct. 2021 | 17 Nov. 2021 | 3 Jan. 2022 |

| | | | Timepoint |
|---|---|---|---|
| | | | 3 Month |
| | | | Project |
| | | | 10275-023 |
| | | | Pull Date |
| | | | 5 Jan. 2022 |
| | | | Batch |
| | | | TB382444 |
| Test | Method | Acceptance Criteria | Results |
| Visual | 25481 | Report results | Packaging: Brown glass vial with a legible label, |
| Appearance | | | metal crimp seal, blue flip off cap, and grey |
| | | | rubber stopper Solution: Clear, colorless liquid, |
| | | | free of visible particulates |
| | | Tested | 28 Jan. 2022 (Packaging)†; |
| | | | 30 Jan. 2022 (Solution)† |
| pH | 05627 | Report Results | 4.6 |
| | | Tested | 30 Jan. 2022 |
| HPLC-UV | 78917 | 90.0-110.0% (% LC) | 104.7 |
| Assay | | Tested | 30 Jan. 2022 |
| Related | 78917 | Individual Impurity: Report Results (RRT/%) | 1.01/0.3 |
| Substances | | Total Impurities: Report Results (%) | 0.3 |
| | | Purity: Report Results (%) | 100 |
| | | Tested | 30 Jan. 2022 |
| UV-Vis | 78933 | 90.0-110.0% (% LC) | Not Scheduled |
| Assay | | Tested | |
| Osmolality | 69153 | Report Result (mOsm/kg) | 486 |
| | | Tested | 17 Jan. 2022 |

TABLE 21
Stability Summary of 81.4 mg/mL of SEQ ID NO: 1 Solution Stored at 40° C. and 75% RH

| | | | Timepoint | | |
|---|---|---|---|---|---|
| | | | 1 Month | 2 Month | 3 Month |
| | | | Project | | |
| | | | 10275-021 | 10275-022 | 10275-023 |
| | | | Pull Date | | |
| | | | 5 Nov. 2021 | 6 Dec. 2021 | 5 Jan. 2022 |
| | | | Batch | | |
| | | | TB378318 | TB380497 | TB382448 |
| Test | Method | Acceptance Criteria | Results | Results | Results |
| Visual | 25481 | Report results | Clear, yellow liquid | Amber glass vial with aluminum | Packaging: Brown glass vial with a |
| Appearance | | | | seal, legible label, and blue flip | legible label, metal crimp seal, |
| | | | | off cap over a gray stopper containing | blue flip off cap, and grey rubber |
| | | | | a clear yellow liquid, visibly free of | stopper Solution: Clear, yellow |
| | | | | particulate matter. | liquid, free of visible particulates |
| | | Tested | 17 Nov. 2021 | 3 Jan. 2022 | 28 Jan. 2022 (Packaging)†; |
| | | | | | 30 Jan. 2022 (Solution)† |
| pH | 05627 | Report Results | 4.9 | 4.9 | 4.9 |
| | | Tested | 19 Nov. 2021 | 3 Jan. 2022 | 30 Jan. 2022 |
| HPLC-UV | 78917 | 90.0-110.0% (% LC) | 102.5 | 102.6 | 92.0♦ |
| Assay | | Tested | 1 Dec. 2021 | 14 Dec. 2021 | 30 Jan. 2022 |
| Related | 78917 | Individual Impurity: | 1.02/1.4 | 0.95/0.2 | 0.88/0.4 |
| Substances | | Report Results (RRT/%) | 1.31/0.3 | 0.97/0.2 | 0.90/0.2 |
| | | | | 1.02/1.6 | 0.99/0.5 |
| | | | | 1.32/0.4 | 0.99/0.4 |
| | | | | | 1.01/0.4 |
| | | | | | 1.02/0.2 |
| | | | | | 1.06/0.5 |
| | | Total Impurities: | 1.7 | 2.4 | 2.6 |
| | | Report Results (%) | | | |
| | | Purity: Report Results (%) | 98 | 98 | 97 |
| | | Tested | 1 Dec. 2021 | 14 Dec. 2021 | 30 Jan. 2022 |
| UV-Vis | 78933 | 90.0-110.0% (% LC) | Not Scheduled | Not Scheduled | Not Scheduled |
| Assay | | Tested | | | |
| Osmolality | 69153 | Report Result (mOsm/kg) | 489 | 491 | 492 |
| | | Tested | 17 Nov. 2021 | 3 Jan. 2022 | 17 Jan. 2022 |

### Example 5: Basic Buffer Stability & Impurities

This example illustrates the impurity of the right shoulder at RRT 1.02 from purity HLPC.

Tables 22-27 show 6 peptide formulations that were stored at a pH of 7.4±0.2 for 197 days. The impurity at RRT 1.02 was initially hypothesized as the loss or cleavage of the Arg functional group (Des-Arg). Additional impurity peaks may arise from Des-Arg, deamidation (conversion of the C-terminal —CONH₂ to —CO₂H), or deamination (conversion of at least one Arg group into citrulline), or a combination at two or more processes.

TABLE 22
SEQ ID NO: 1 Formulation (1.0 mg/mL) in 20 mM Tromethamine buffer stored at 25° C.
Mass Table with relative abundance >50%

| | | |
|---|---|---|
| PPL1954 | MW average | 3399.2 |

QCID# 6916, PPL1954//9620-4-2, 25° C., 1.0 mg/mL in 20 mM Tromethamine buffer

| right shoulder | Observed | Deconvoluted | | | Mass |
|---|---|---|---|---|---|
| Peak # | m/z | Mass (Da) | Ion/Charge State | comments | difference |
| 1 | 811.7 | 3242.8 | [M + 4H]4+ | des-Arg | −156.4 |
| 2 | 1119.4 | 3242.2 | [M + 2H + TFA]3+ | des-Arg | −157.0 |
| 3 | 850.6 | 3398.4 | [M + 4H]4+ | Deamidation/Deimination | Mass as parent |
| 4 | 1133.9 | 3398.7 | [M + 3H]3+ | Deamidation/Deimination | Mass as parent |
| 5 | 1082.3 | 3243.9 | [M + 3H]3+ | des-Arg | −155.3 |
| 6 | 1171.4 | 3397.2 | [M + 3H + TFA]3+ | Deamidation/Deimination | Mass as parent |
| 7 | 839.8 | 3241.2 | [M + 4H + TFA]4+ | des-Arg | −158.00 |

TABLE 23
SEQ ID NO: 1 Formulation (1.0 mg/mL) in 0.1M Glycine buffer stored at −20° C.
Mass Table with relative abundance >50%

| | | |
|---|---|---|
| PPL1954 | MW average | 3399.2 |

QCID# 6917, PPL1954//9620-5-2, −20° C., 1.0 mg/mL in 0.1M Glycine buffer

| right shoulder | Observed | Deconvoluted | | | Mass |
|---|---|---|---|---|---|
| Peak # | m/z | Mass (Da) | Ion/Charge State | comments | difference |
| 1 | 811.7 | 3242.8 | [M + 4H]4+ | des-Arg | −156.4 |
| 2 | 839.9 | 3241.6 | [M + 4H + TFA]4+ | des-Arg | −157.6 |
| 3 | 878.6 | 3396.4 | [M + 4H + TFA]4+ | Deamidation/Deimination | Mass as parent |
| 4 | 1081.8 | 3242.4 | [M + 3H]3+ | des-Arg | −156.8 |
| 5 | 1119.3 | 3241.9 | [M + 2H + TFA]3+ | des-Arg | −157.30 |
| 6 | 1157.2 | 3243.6 | [M + 3H + 2TFA]3+ | des-Arg | −155.6 |
| 7 | 1171.5 | 3397.5 | [M + 3H + TFA]3+ | Deamidation/Deimination | Mass as parent |

TABLE 24
SEQ ID NO: 1 Formulation (1.0 mg/mL) in 0.1M Glycine buffer stored at 25° C.
Mass Table with relative abundance >50%

| | | |
|---|---|---|
| PPL1954 | MW average | 3399.2 |

QCID# 6918, PPL1954//9620-5-2, 25° C., 1.0 mg/mL in 0.1M Glycine buffer

| right shoulder | Observed | Deconvoluted | | | Mass |
|---|---|---|---|---|---|
| Peak # | m/z | Mass (Da) | Ion/Charge State | comments | difference |
| 1 | 811.7 | 3242.8 | [M + 4H]4+ | des-Arg | −156.4 |
| 2 | 839.8 | 3241.2 | [M + 4H + TFA]4+ | des-Arg | −158.0 |
| 3 | 1081.8 | 3242.4 | [M + 3H]3+ | des-Arg | −156.8 |
| 4 | 1119.3 | 3241.9 | [M + 2H + TFA]3+ | des-Arg | −157.30 |
| 5 | 1134 | 3399 | [M + 3H]3+ | Deamidation/Deimination | Mass as parent |
| 6 | 1171.5 | 3397.5 | [M + 3H + TFA]3+ | Deamidation/Deimination | Mass as parent |

TABLE 25
SEQ ID NO: 1 Formulation (1.0 mg/mL) in 20 mM Tromethamine buffer stored at −20° C.
Mass Table with relative abundance >50%

| | | |
|---|---|---|
| PPL1954 | MW average | 3399.2 |

QCID# 6915, PPL1954//9620-4-2, −20° C., 1.0 mg/mL in 20 mM Tromethamine buffer

| right shoulder | Observed | Deconvoluted | | | Mass |
|---|---|---|---|---|---|
| Peak # | m/z | Mass (Da) | Ion/Charge State | comments | difference |
| 1 | 811.6 | 3242.4 | [M + 4H]4+ | des-Arg | −156.8 |
| 2 | 839.8 | 3241.2 | [M + 4H + TFA]4+ | des-Arg | −158.00 |
| 3 | 850.9 | 3399.6 | [M + 4H]4+ | Deamidation/Deimination | Mass as parent |
| 4 | 878.9 | 3397.6 | [M + 4H + TFA]4+ | Deamidation/Deimination | Mass as parent |
| 5 | 1081.7 | 3242.1 | [M + 3H]3+ | des-Arg | −157.1 |
| 6 | 1119.3 | 3241.9 | [M + 2H + TFA]3+ | des-Arg | −157.3 |
| 7 | 1133.9 | 3398.7 | [M + 3H]3+ | Deamid ation/Deimination | Mass as parent |
| 8 | 1157.4 | 3241.2 | [M + 3H + 2TFA]3+ | des-Arg | −158.0 |

TABLE 26
SEQ ID NO: 1 Formulation (1.0 mg/mL) in PBS solution stored at −20° C.
Mass Table with relative abundance >50%

| | | |
|---|---|---|
| PPL1954 | MW average | 3399.2 |

QCID# 6914, PPL1954//9620-3-2, −20° C., 1.0 mg/mL in PBS solution

| right shoulder | Observed | Deconvoluted | | | Mass |
|---|---|---|---|---|---|
| Peak # | m/z | Mass (Da) | Ion/Charge State | comments | difference |
| 1 | 811.5 | 3242 | [M + 4H]4+ | des-Arg | −157.2 |
| 2 | 839.6 | 3240.4 | [M + 4H + TFA]4+ | des-Arg | −158.8 |
| 3 | 850.7 | 3398.8 | [M + 4H]4+ | Deamidation/Deimination | Mass as parent |
| 4 | 868.3 | 3241.2 | [M + 4H + 2TFA]4+ | Deamidation/Deimination | −158.00 |
| 5 | 879.0 | 3398 | [M + 4H + TFA]4+ | Deamidation/Deimination | Mass as parent |
| 6 | 1081.8 | 3242.4 | [M + 3H]3+ | des-Arg | −156.7 |
| 8 | 1119.5 | 3242.5 | [M + 2H + TFA]3+ | des-Arg | −156.70 |
| 9 | 1157.7 | 3245.1 | [M + 3H + 2TFA]3+ | des-Arg | −154.1 |

TABLE 27
SEQ ID NO: 1 Formulation (1.0 mg/mL) in PBS solution stored at 25° C.
Mass Table with relative abundance >50%

| | | |
|---|---|---|
| PPL1954 | MW average | 3399.2 |

QCID# 6913, PPL1954//9620-3-2, 25° C., 1.0 mg/mL in PBS solution

| right shoulder | Observed | Deconvoluted | | | Mass |
|---|---|---|---|---|---|
| Peak # | m/z | Mass (Da) | Ion/Charge State | comments | difference |
| 1 | 811.7 | 3242.8 | [M + 4H]4+ | des-Arg | −156.4 |
| 2 | 839.8 | 3241.2 | [M + 4H + TFA]4+ | des-Arg | −158.0 |
| 3 | 868.3 | 3241.2 | [M + 4H + 2TFA]4+ | des-Arg | −158.0 |
| 4 | 1081.7 | 3242.1 | [M + 3H]3+ | des-Arg | −157.1 |
| 5 | 1119.3 | 3241.9 | [M + 2H + TFA]3+ | des-Arg | −157.30 |
| 6 | 1157.4 | 3241.2 | [M + 3H + 2TFA]3+ | des-Arg | −158.00 |
| 7 | 1171.3 | 3396.9 | [M + 3H + TFA]3+ | Deamidation/Deimination | Mass as parent |

### Example 6: pH Solubility of Peptide Formulations

This example illustrates the near saturation ability of exemplary peptides described here.

SEQ ID NO: 1 was dissolved to near saturation of different pH values. The formulations had a pH value of 5.0±0.1, 7.4±0.1, or 8.0±0.1.

### Experimental Procedure

396.69 mg of SEQ ID NO:1 was weighed out and dissolved in approximately 3 mL of HPLC-grade water resulting in a clear and colorless solution. The pH was 4.80 and the concentration approximately 132 mg/mL. The pH was adjusted with 0.08 M NaOH to pH=4.99. 1.1 mL was pulled, transferred to a serum vial, frozen and lyophilized. The remaining solution was adjusted to pH=7.40 with 0.08 M and 1 M NaOH. 1.45 mL was pulled, transferred to a serum vial, frozen and lyophilized. The remaining solution was adjusted to pH=8.00 with 0.08 M NaOH. This remaining solution (˜1.9 mL) was transferred to a serum vial, frozen and lyophilized. After lyophilization 200 μL HPLC-grade water was added to the solids in the serum vials and a colorless slurry was obtained. An additional 200 μL HPLC-grade water was added to obtain clear and colorless solutions. At pH=5.0 the solids went into solution without foaming; solids at pH=7.4 and pH=8 took longer to dissolve. These solutions (0.4 mL) were transferred to conical Eppendorf tubes. The vials were washed with 0.2 mL of HPLC grade water and the washes were combined with the respective solutions at the three different pH conditions. The samples were frozen and lyophilized. To the lyo cakes 50 μL increments of HPLC grade water was added. Transition from suspension or slurry to viscous solution was hard to capture. After adding 150 μL to 350 μL of HPLC grade water and intermittent mixing and storage at ambient temperature viscous and clear solutions were obtained which turned to gels when stored refrigerated at 2-8° C. All viscous solutions were centrifuged for 10 min at 12 krpm. No visible pellets were detected after centrifugation. Supernatants of the samples were analyzed for peptide concentration and purity by HPLC analysis.

TABLE 28
HPLC Method Information

| | |
|---|---|
| Column | YMC Pack ODS-A, 250 × 4.6 mm, S-5 μm, 12 nm |
| Mobile Phase | A: 0.1% Trifluoroacetic acid in Water, |
| | B: 0.1% Trifluoroacetic Acid in Acetonitrile |
| Gradient | 20% to 50% B in 27 min |
| Column | 40° C. |
| Temperature | |
| Flow Rate | 0.5 mL/min |
| Injection Volume | 10 μL |

TABLE 29
Solubility of near saturation for SEQ
ID NO: 1 at pH 5.0, 7.4, and 8.1

| pH | Concentration (mg/mL) | Purity (%) |
|---|---|---|
| 5.0 | 328 | 95.6 |
| 7.4 | 228 | 97.2 |
| 8.1 | 250 | 96.7 |

The solubility near saturation concentrations of SEQ ID NO: 1 in water at pH=5.0 was found to be 328 mg/mL, at pH=7.4 it was 228 mg/mL and at pH=8.1 it was 250 mg/mL as seen in Table 29. All three solutions are viscous and refrigerated at 2-8 ° C. form gels.

### Example 7: Peptide Formulation Stability

This example illustrates the stability of exemplary peptides formulations described herein under 40° C. over a period of time.

SEQ ID NO: 1 was dissolved in sterile water for injection resulting solutions at a concentration of 1 mg/mL and pH adjusting agents were added to achieve a pH value in the range of 4-7. The pH adjusting chosen were acetate and sodium hydroxide and citric acid (for pH 4). Each of the solutions were filtered through a 0.45 um filter and samples were stored in 1 mL aliquots at 40° C., room temperature (approximately 20° C.), refrigerated conditions (5° C.), and frozen at −20° C. Samples were withdrawn at regular intervals (t=0, and Days 2, 7, 14, and 28) and analyzed by HPLC to determine if chemical degradation of the peptide could be detected in any samples. Degradation rates between samples of each pH were also analyzed.

TABLE 30
1 mg mL SEQ ID NO: 1 Samples with varying pH

| Sample | pH | Solution Preparation |
|---|---|---|
| 1 | 4 | 18.3 mM acetic acid |
| 2 (citrate) | 4 | 2.6 mM citric acid |
| 3 | 5 | 17.4 mM acetic acid |
| 4 | 6 | 425 μM NaOH |
| 5 | 7 | 800 μM NaOH |
| 6 (analytical standard) | NA | In Mobile Phase A solution |

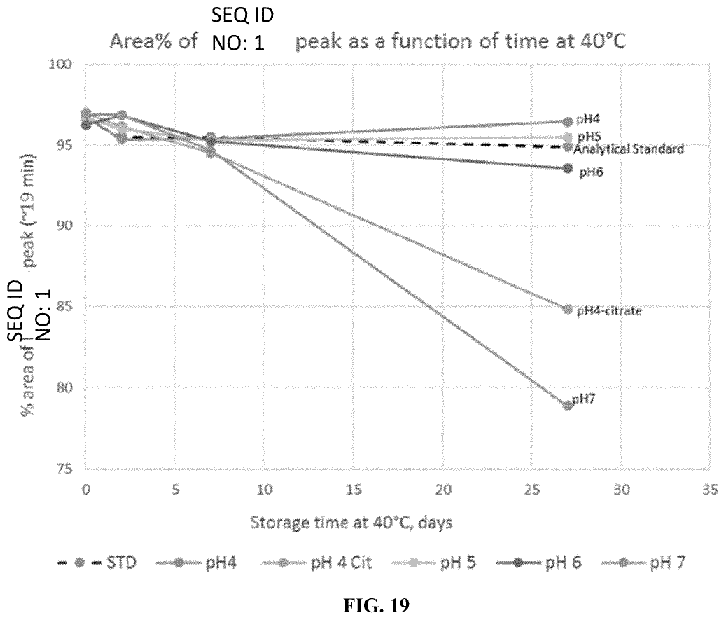

The % area of SEQ ID NO: 1 as a function of time over the stored period was measured. FIG. 19 shows the % area over the stored period of the 40° C. After 7 days of storage at 40° C., differences were negligible between samples and there was no significant evidence of impurity increase in any of the samples. After 27 days of storage, samples that had been prepared at pH 7 and at pH 4 with citrate exhibited new peaks or an increase of existing peaks and/or shoulders indicating an increase in impurities. Overall, maximum stability was observed at pH 4 and pH 5 made with acetic acid.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A pharmaceutical formulation comprising:
(a) a peptide or pharmaceutically acceptable salt thereof comprising at least about 70% sequence identity to a polypeptide sequence of:
Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1)
Arg-Val-Val-Arg-Val-Val-Arg-Arg-Val-Val-Arg-Arg (SEQ ID NO: 15);
Arg-Val-Val-Arg-Val-Val-Arg-Arg-Trp-Val-Arg-Arg (SEQ ID NO: 16);
Arg-Trp-Trp-Arg-Trp-Trp-Arg-Arg-Trp-Trp-Arg-Arg (SEQ ID NO: 17);
Trp-Arg-Arg-Trp-Trp-Arg-Arg-Trp-Trp-Arg-Trp-Trp-Arg-Arg-Trp-Trp-Arg-Arg (SEQ ID NO: 18);
Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19);
Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20);
Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21);
Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22);
Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23);
Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); or Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25); and
(b) at least one pharmaceutically acceptable excipient; wherein the pharmaceutical formulation has a pH of about 3.5 to about 5.5; and
wherein the pharmceutical formulation comprises at most 5% by weight of at least one impurity as measured by high-performance liquid chromatography (HPLC) when stored for at least 50 days at 40° C.

2. The pharmaceutical formulation of claim 1, wherein the pharmaceutical formulation is stable for at least about 3 months at a temperature of about 40° C.

3. The pharmaceutical formulation of claim 1, wherein the pharmaceutical formulation is stable for at least about 6 months at a temperature of about 40° C.

4. The pharmaceutical formulation of claim 1, wherein the pharmaceutical formulation is stable for at least about 9 months at a temperature of about 40° C.

5. The pharmaceutical formulation of claim 1, wherein the pharmaceutical formulation comprises a pH of about 5.0.

6. The pharmaceutical formulation of claim 1, wherein the pharmaceutical formulation comprises a pH of about 4.5.

7. The pharmaceutical formulation of claim 1, wherein the pharmaceutical formulation comprises a pH of about 4.9.

8. The pharmaceutical formulation of claim 1, wherein the pharmaceutical formulation comprises at most 3.5% by weight of said at least one impurity as measured by HPLC.

9. The pharmaceutical formulation of claim 1, wherein the pharmaceutical formulation comprises at most 2% by weight of said at least one impurity as measured by HPLC.

10. The pharmaceutical formulation of claim 1, wherein the pharmaceutical formulation comprises at most 1.5% by weight of said at least one impurity measured by HPLC.

11. The pharmaceutical formulation of claim 1, wherein the pharmaceutical formulation comprises at most 3% by weight of said at least one impurity as measured by UV-Vis spectroscopy.

12. The pharmaceutical formulation of claim 1, wherein the peptide is present at a concentration of about 10 mg/mL to about 100 mg/mL.

13. The pharmaceutical formulation of claim 12, wherein the peptide is present at a concentration at about 15 mg/mL, about 30 mg/mL, about 40 mg/mL, about 70 mg/mL, or about 80 mg/mL.

14. The pharmaceutical formulation of claim 1, wherein the peptide comprises at least about 90% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1).

15. The pharmaceutical formulation of claim 14, wherein the peptide comprises Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1).

16. The pharmaceutical formulation of claim 1, wherein the excipient comprises an isotonicity agent.

17. The pharmaceutical formulation of claim 16, wherein the isotonicity agent is sodium chloride.

18. The pharmaceutical formulation of claim 1, further comprising a pH adjustment agent.

19. The pharmaceutical formulation of claim 18, wherein the pH adjustment agent comprises hydrochloric acid, sodium hydroxide, or any combination thereof.

20. The pharmaceutical formulation of claim 1, further comprising a pH buffering agent.

21. The pharmaceutical formulation of claim 20, wherein the pH buffering agent is selected from the group consisting of sodium hydrogen phosphate, sodium dihydrogen phosphate, potassium dihydrogen phosphate, potassium hydrogen phosphate, glycine, tris(hydroxymethyl)aminomethane, and any combination thereof.

22. The formulation of claim 1, wherein the formulation is free of a buffering agent.

23. The formulation of claim 1, wherein the formulation comprises an osmolality of at least about 30 milliosmoles per kilogram (mOsm/kg) to at least about 800 mOsm/kg.

24. The formulation of claim 23, wherein the formulation comprises an osmolality of about 100 mOsm/kg to about 500 mOsm/kg.

25. A kit comprising: (i) the pharmaceutical formulation of claim 1; (ii) an aqueous carrier, wherein the aqueous carrier is sterile water for injection; (iii) a mixing container; and (iv) instructions for use.

26. The kit of claim 25, wherein kit further comprises (v) a second aqueous carrier, wherein the second aqueous carrier is aqueous sodium bicarbonate.

27. A method of preventing or treating an infection in a subject in need thereof, wherein the method comprising locally administering of the pharmaceutical formulation of claim 1 to a site of infection, wherein administration comprises washing, irrigating, debridement, or a combination thereof of the site of infection, thereby preventing or treating the infection.

28. The method of claim 27, wherein the infection is periprosthetic joint infection.

29. The method of claim 27, wherein the infection is a bacterial infection, wherein the bacterial species is selected from the group consisting of: Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, and any combination thereof.

30. A method of preventing or treating an infection in a subject in need thereof, wherein the method comprising locally administering the kit of claim 25 to a site of infection, wherein administration comprises washing, irrigating, debridement, or a combination thereof of the site of infection, thereby preventing or treating the infection.
